(19) United States
(12) Patent Application Publication
(10) Pub. No.: US 2024/0058458 A1
(43) Pub. Date: Feb. 22, 2024

(54) METHODS, COMPOUNDS, AND COMPOSITIONS FOR MODIFYING CAR-T CELL ACTIVITY

(71) Applicant: Purdue Research Foundation, West Lafayette, IN (US)

(72) Inventors: Philip S. LOW, West Lafayette, IN (US); John Victor NAPOLEON, West Lafayette, IN (US); Qian LUO, West Lafayette, IN (US)

(21) Appl. No.: 17/905,748

(22) PCT Filed: Mar. 5, 2021
(86) PCT No.: PCT/US21/21201

§ 371 Date: Sep. 6, 2022

Related U.S. Application Data
(60) Provisional application No. 62/986,349, filed on Mar. 6, 2020.

(51) Int. Cl. A61K 47/54; A61K 39/00; A61K 47/55; A61K 47/60

(52) U.S. Cl. CPC A61K 47/555; A61K 39/4611; A61K 39/4631; A61K 39/4646; A61K 47/551; A61K 47/558; A61K 47/60; A61K 2239/39

(57) ABSTRACT

Compounds, compositions and methods for reducing off-target toxicity of T cells expressing a chimeric antigen receptor (CAR-T cells) and/or providing enhanced control of CAR-T cell activation, and methods of treating a subject and/or modifying CAR-T cell activity in a subject with cancer.

### PRIORITY

This patent application is related to and claims the priority benefit of U.S. Provisional Patent Application No. 62/986,349, filed Mar. 6, 2020, the content of which is hereby incorporated by reference in its entirety.

### FIELD

This disclosure relates to compounds, compositions, and methods for treating a subject with cancer and reducing off-target toxicity of T cells expressing chimeric antigen receptors (CAR-T cells) and/or providing enhanced control of CAR-T cell activation. Particularly, targeting moieties are employed with active compounds in combination with CAR-T cell therapies to direct the CAR-T cells and modify the activity thereof as desired.

### BACKGROUND

Immunotherapy based on adoptive transfer of lymphocytes (e.g., T cells) into a patient is a valuable therapy in the treatment of cancer and other diseases. Important advancements have been made in the development of immunotherapies based on adoptive transfer of lymphocytes. Among the many different types of immunotherapeutic agents, one of the most promising of the immunotherapeutic agents being developed is T cells expressing chimeric antigen receptors (CAR-T cells).

T cells have been genetically engineered to produce artificial T cell receptors on their surface called chimeric antigen receptors, or CARs. CARs are proteins that allow T cells to recognize a specific, pre-selected protein, or antigen, found on targeted tumor cells. CAR-T cells can be cultured and expanded in the laboratory, then re-infused into the autologous subject. Through the guidance of the engineered T cell receptor, CAR-T cells recognize and destroy the cancer cells that display the specific antigen on their surfaces.

Conventional CARs include a recognition region (e.g., a single chain fragment variable (scFv) region derived from an antibody) for recognition and binding to the antigen expressed by the tumor. The recognition region can be fused to the exoplasmic domain of a T cell receptor to enhance engagement of the T cell with the cancer cell. To facilitate rapid killing of the cancer cell, the CAR can be further modified to contain an activation signaling domain that, for example, can be derived from CD3ζ, a Fc receptor gamma signaling domain, or one or more costimulatory domains such as CD28, 4-1BB, ICOS, OX40, etc.

Although CAR-T cells have shown positive results in vitro, use of these genetically engineered T cells to treat human cancers has introduced challenges associated with control of the CAR-T cell's activity. One such challenge, especially in solid tumor cancers, is identifying target antigens expressed homogeneously throughout the tumor or other malignant target and not on normal tissues. To date, few targets with homogenous expression on epithelial cancers have been identified. Accordingly, CAR-T cells can potentially damage normal tissues by targeting a tumor-associated antigen that is also expressed on normal tissues. This can lead to toxicities that can harm or even kill the cancer patient. Such toxicities can include, for example, neurologic toxicity, “on target/off tumor” recognition, and anaphylaxis.

Additionally, cytokine-associated toxicity, also referred to as a “cytokine storm” or cytokine release syndrome (CRS), is a common and potentially lethal complication of CAR-T cell therapy. CRS is a non-antigen specific toxicity that can occur as a result of the high-levels of CAR-T cell expansion and immune activation typically required to mediate clinical benefit using modem immunotherapies such as CAR-T cell transfer. Interaction between conventional CAR-T cells and its target causes the activation and expansion of the CAR-T cells and lysis of both normal and tumor cells. This is associated with the release of several cytokines such as interferon gamma (IFN-γ) and tumor-necrosis factor alpha (TNFα). The combination of these signals also triggers the activation of monocytes and macrophages with enhanced tumoricidal capacity and that secrete high levels of pro-inflammatory cytokines (e.g., interleukin 6 (IL-6), interleukin 1 (IL-1), and interleukin 10 (IL-10)) and other mediators such as inducible nitric oxide synthase (iNOS), all of which can promote the progression of CRS and other related toxicities. In most patients, CRS symptoms are usually mild and flu-like, with fevers and myalgias. However, some patients experience a severe inflammatory syndrome that can result in multiorgan system failure.

There remains a need to provide improved methods and compositions for CAR-T cell therapies, particularly to abrogate toxicity related to these medical interventions. While CAR-T cells show great promise as a tool in the treatment of diseases, such as cancer, additional CAR-T cell therapies are needed that provide reduced off-target toxicity and more precise control of CAR-T cell activation.

### SUMMARY

Compounds, compositions and methods for reducing off-target toxicity, and more precisely controlling activation of T cells that express chimeric antigen receptors (CAR-T cells), which advance CAR-T cell therapy (e.g., treating cancer), are provided. In such a therapy, a small molecule ligand linked to a first targeting moiety (by a first linker or directly) is used as an adaptor compound (i.e., a bridge) between one or more cancer cells and CAR-T cells that, in use, directs the CAR-T cells to the cancer for treatment of the cancer. The small molecule ligand that is part of the adaptor compound can be, for example, a folate, a 2-[3-(1,3-dicarboxypropyl)ureido]pentanedioic acid (DUPA) ligand, a neurokinin 1 receptor (NK-1R) ligand, a carbonic anhydrase IX (CAIX) ligand, a ligand of gamma glutamyl transpeptidase, a natural killer group 2D receptor (NKG2D) ligand, or a cholecystokinin B receptor (CCKBR or CCK2) ligand, each of which is a small molecule ligand that binds specifically a receptor that is overexpressed on certain types of cancer cells (i.e., the receptor for these ligands is overexpressed on the target cancer cells as compared to normal tissues and non-target cancer cells).

In the adaptor compound, the small molecule ligand can be linked to a first targeting moiety that binds to the chimeric antigen receptors (CAR) expressed by CAR-T cells. The first “targeting moiety” can be selected, for example, from 2,4-dinitrophenol (DNP), 2,4,6-trinitrophenol (TNP), biotin, digoxigenin, fluorescein, fluorescein isothiocyanate (FITC), NHS-fluorescein, pentafluorophenyl ester (PFP), tetrafluorophenyl ester (TFP), a knottin, a centyrin, and a designed ankyrin repeat protein (DARPin).

The first targeting moiety binds to the recognition region of the genetically engineered CAR expressed by CAR-T cells. Accordingly, the recognition region of the CAR (e.g., a single chain fragment variable region (scFv) of an antibody, a Fab, Fv, Fc, (Fab′)₂ fragment, and the like) is directed to the first targeting moiety. Thus, the adaptor compound comprising the small molecule ligand linked to the first targeting moiety acts as a bridge between the cancer and the CAR-T cells, directing the CAR-T cells to the cancer for treatment of the cancer. Accordingly, this CAR-T cell therapy can provide reduced off-target toxicity, and more precise control of CAR-T cell activation because the small molecule ligand in the adaptor compound binds specifically to targeted cancer cells and not to non-targeted tissues resulting in reduced off-target toxicity, and the adaptor compound has a short half-life in circulation which allows for rapid changes in the concentration of the adaptor compound to control precisely CAR-T cell activation.

In certain embodiments, at least one of the one or more adaptor compounds, or the pharmaceutically acceptable salts thereof, is not an antibody nor comprises a fragment of an antibody.

The therapy described above also has the advantage that a cocktail of different tumor-specific adaptor compounds (e.g., different small molecule ligands but the same targeting moiety) can be administered. As a result, heterogeneous solid tumors that have mutated and have lost their primary tumor antigen can still be eradicated because multiple tumor-specific adaptor compounds are used and a different tumor-specific adaptor compound(s) can act as a bridge(s) between the cancer and the CAR-T cells when a tumor mutates and loses its primary tumor antigen.

The use of adaptor compounds also provides ‘universality’ because a single type of CAR-T cell, with a single type of recognition region, can be used to eradicate multiple tumor types. The CAR-T cell has a single type of recognition region directed to the first targeting moiety in the adaptor compounds, but different adaptor compounds have different small molecule ligands (i.e., tumor targeting ligands) directed to multiple tumor types. Thus, the adaptor compounds make the CAR-T cells ‘universal’ CAR-T cells for killing tumors that express different antigens because the different small molecule ligands in the adaptor compounds bind to different tumor types, but only one type of CAR-T cell with one type of recognition region is used.

Additional problems faced in CAR-T cell therapies are that CAR-T cells may become dysfunctional or “exhausted” or reduced proliferation may result upon chronic exposure to tumor antigens or immunosuppressive factors (e.g., myeloid-derived suppressor cells (MDSCs), tumor-associated macrophages (TAMs), regulatory T cells (Tregs), and inhibitory cytokines) in the tumor microenvironment. In contrast, CAR-T cells may become overactive resulting in unwanted side effects of CAR-T cell therapies, such as cytokine release syndrome (CRS), which may be fatal to the patient. To address these problems, in at least one embodiment, the endocytosis of the recognition region (e.g., a scFV fragment) that is part of the CAR is exploited to deliver an activity modifying compound, or a pharmaceutically acceptable salt thereof, to CAR-T cells.

The activity modifying compound, or a pharmaceutically acceptable salt thereof, is linked to a second targeting moiety, either directly or by a second linker. In certain embodiments, the second targeting moiety is selected from a group consisting of: DNP, TNP, biotin, digoxigenin, fluorescein, FITC, NHS-fluorescein, pentafluorophenyl ester, tetrafluorophenyl ester, knottin, centyrin, and DARPin. In some embodiments, one or both of the first targeting moiety and the second targeting moiety do not comprise a peptide epitope.

The activity modifying compound, or a pharmaceutically acceptable salt thereof, can comprise a rejuvenating compound, or a pharmaceutically acceptable salt thereof. The rejuvenating compound, or the pharmaceutically acceptable salt thereof, can be a compound, drug or active agent formulated to rejuvenate exhausted CAR-T cells (e.g., by blocking inhibitory signaling or exhausted CAR-T cells, reactivating the exhausted CAR-T cells through an antigen independent pathway, performing both of the foregoing, or via other methods). In various embodiments, the rejuvenating compound, or the pharmaceutically acceptable salt thereof, is selected from a group consisting of a Toll-Like Receptor (TLR) agonist (e.g., a TLR1, TLR2, TLR7, TLR8, TLR7/8, TLR9, TLR3, TLR4, etc.), a stimulator of interferon genes (STING) agonist, a Nod-like receptor stimulant (NLRs), an absent in melanoma 2 (AIM2)-like receptor (ALRs) agonist, a kinase inhibitor targeting kinases such as GSK-3beta, PI3K, etc., and a phosphatase inhibitor. In additional embodiments, for example where the rejuvenating compound or the pharmaceutically acceptable salt thereof is a TLR7/8 agonist, the rejuvenating compound or pharmaceutically acceptable salt thereof has one of the following formulae:

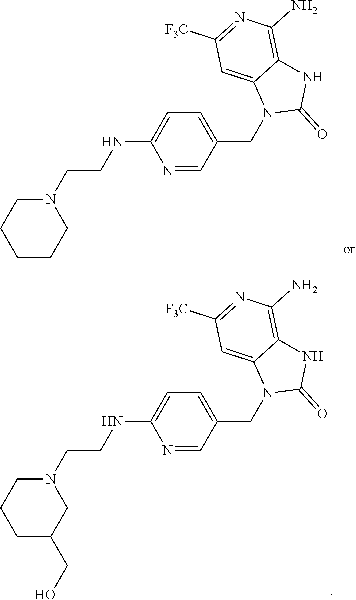 [SMILES: Nc1nc(C(F)(F)F)cc2c1[nH]c(=O)n2Cc1ccc(NCCN2CCCCC2)nc1 ; Nc1nc(C(F)(F)F)cc2c1[nH]c(=O)n2Cc1ccc(NCCN2CCCC(CO)C2)nc1]

When administered, the rejuvenating drug is delivered into the CAR-T cell (e.g., via the second targeting moiety and the CAR recognition region). By concentrating the rejuvenating drug within the CAR-T cell, exhausted or dysfunctional CAR-T cells can be rejuvenated to functional and tumor-killing CAR-T cells, leading to renewed eradication of a solid tumor. The administration of the rejuvenating compounds to target CAR-T cells can reverse exhaustion or dysfunction of CAR-T cells induced by the tumor microenvironment.

In some embodiments, the rejuvenating compound or pharmaceutically acceptable salt thereof has the following formula:

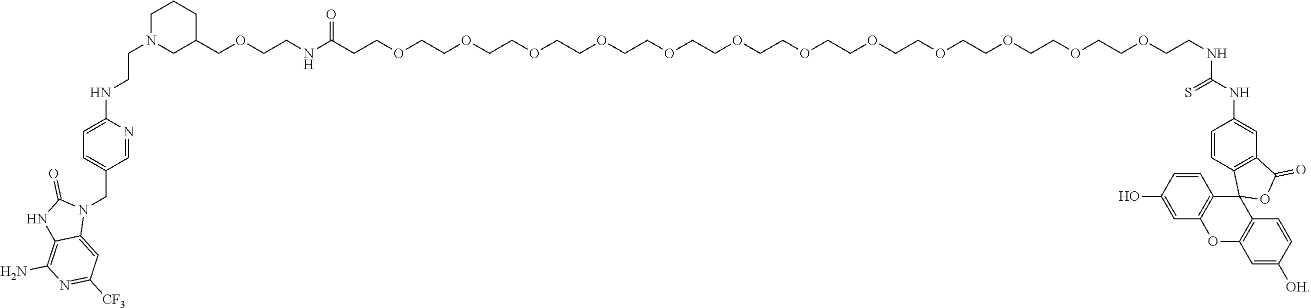 [SMILES: Nc1nc(C(F)(F)F)cc2c1[nH]c(=O)n2Cc1ccc(NCCN2CCCC(COCCNC(=O)CCOCCOCCOCCOCCOCCOCCOCCOCCOCCOCCOCCOCCNC(=S)Nc3ccc4c(c3)C(=O)OC43c4ccc(O)cc4Oc4cc(O)ccc43)C2)nc1]

In some embodiments, the rejuvenating compound or pharmaceutically acceptable salt thereof has a structure of one of the following formulae:

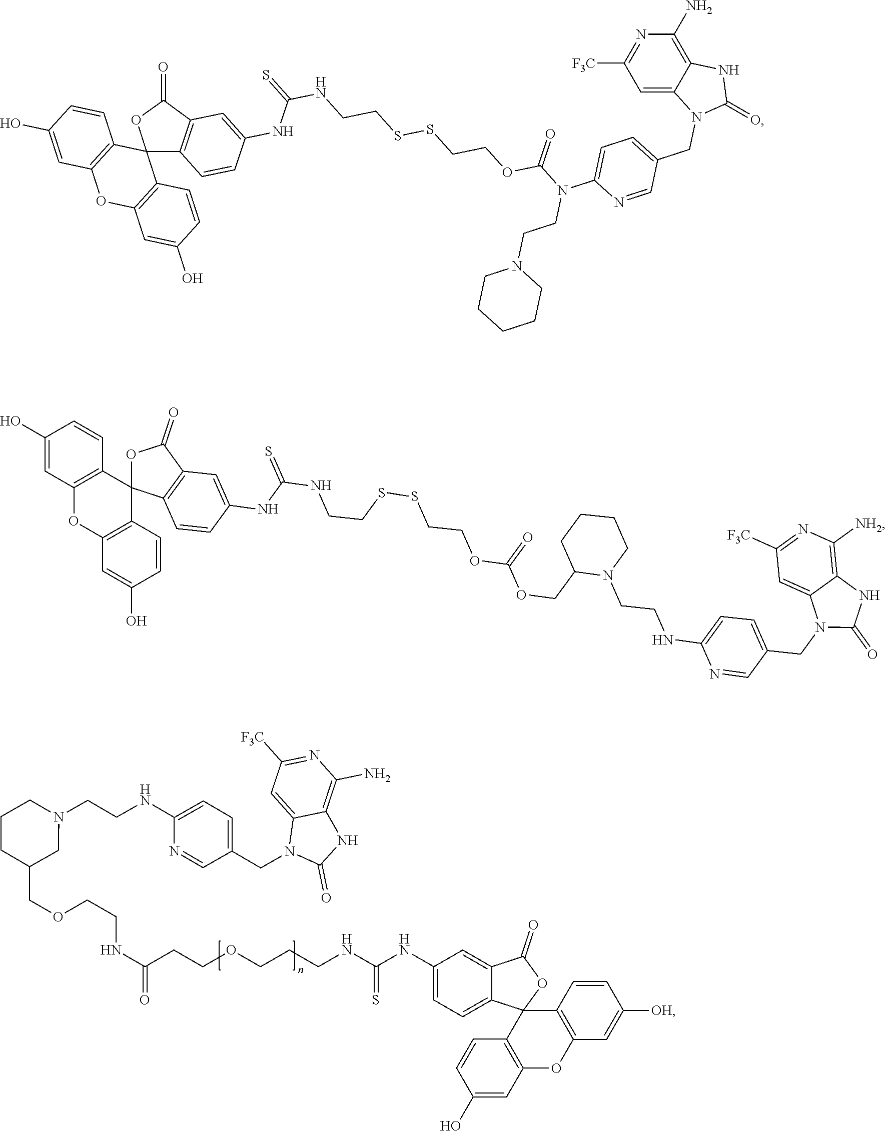

wherein n=0 to 200, or

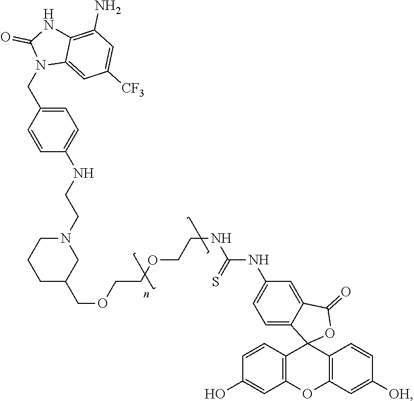

wherein n=0 to 50.

To allow for precise control of CAR-T cell activity and to reduce activity of CAR-T cells, the activity modifying compound, or a pharmaceutically acceptable salt thereof, can comprise an immunosuppressive compound, or a pharmaceutically acceptable salt thereof. The immunosuppressive compound or pharmaceutically acceptable salt thereof can be a compound, drug or active agent formulated to reduce the activity of the CAR-T cells. In various embodiments, the immunosuppressive compound or pharmaceutically acceptable salt thereof is selected from a group consisting of tacrolimus, sirolimus, and cyclosporine. When administered, the immunosuppressive drug is delivered into the CAR-T cell (e.g., via the second targeting moiety and the CAR recognition region). By concentrating the immunosuppressive drug within the CAR-T cell, CAR-T cell activity can be reduced leading to inhibition of adverse side effects of excessive CAR-T cell activation such as CRS.

A method of modifying T cell activity in a subject with cancer (e.g. treating cancer) and/or treatment of a cancer is also provided. The method comprises administering to a subject a composition comprising: a vector (e.g., a lentiviral vector) comprising a promoter operatively linked to a nucleic acid sequence encoding a CAR, or CAR-T cells expressing the CAR, wherein the CAR is directed to a first targeting moiety, a second targeting moiety, or both the first and second targeting moieties; administering to the patient one or more adaptor compounds, or pharmaceutically acceptable salts thereof, wherein each adaptor compound, or pharmaceutically acceptable salt thereof, comprises a small molecule ligand linked to the first targeting moiety; and administering to the subject an activity modifying compound linked to a second targeting moiety (e.g., a rejuvenating compound or a pharmaceutically acceptable salt thereof or an immunosuppressive compound or a pharmaceutically acceptable salt thereof). The method can further comprise imaging the subject prior to administering the one or more adaptor compounds, or the pharmaceutically acceptable salts thereof, or prior to administering the CAR-T cell composition.

The small molecule ligand of each adaptor compound can be linked to the first targeting moiety thereof by a first linker. Independently, the activity modifying compound can be linked to the second targeting moiety by a second linker. In embodiments where both the first and second linkers are employed, the first and second linkers can have the same structure or different structures. The first and second linker can each independently be a releasable linker or a non-releasable linker. In certain embodiments, the first linker, the second linker, or both can independently comprise a C₁-C₂₀ alkyl, a polyethylene glycol (PEG), a polyproline, an oligo-(4-piperidine carboxylic acid, an oligo piperidine, a peptide, a saccharo-peptide, a hydrophilic amino acid, a sugar, an unnatural peptidoglycan, a polyvinylpyrrolidone, pluronic F-127, or a combination thereof. In some embodiments, the first linker or the second linker comprises a PEG.

In certain embodiments, the one or more adaptor compounds, or the pharmaceutically acceptable salts thereof, each have the formula:

B—L—T,

where B represents the small molecule ligand, L represents the first linker, and T represents the first targeting moiety, and L comprises a structure having the formula:

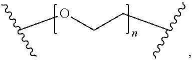

where n is an integer from 0 to 200.

Similarly, in certain embodiments, the second linker can have a structure having the formula:

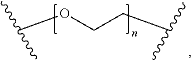

where n is an integer from 0 to 200.

Where employed, the first linker in the one or more adaptor compounds (or pharmaceutically acceptable salts thereof) can be positioned between the small molecule ligand and the first targeting moiety. Similarly, where employed, the second linker in the activity modifying compound can be positioned between the second targeting moiety and the activity modifying compound. In certain embodiments, the first linker and/or the second linker can each comprise a chemical moiety having a structure independently selected from the following formulae:

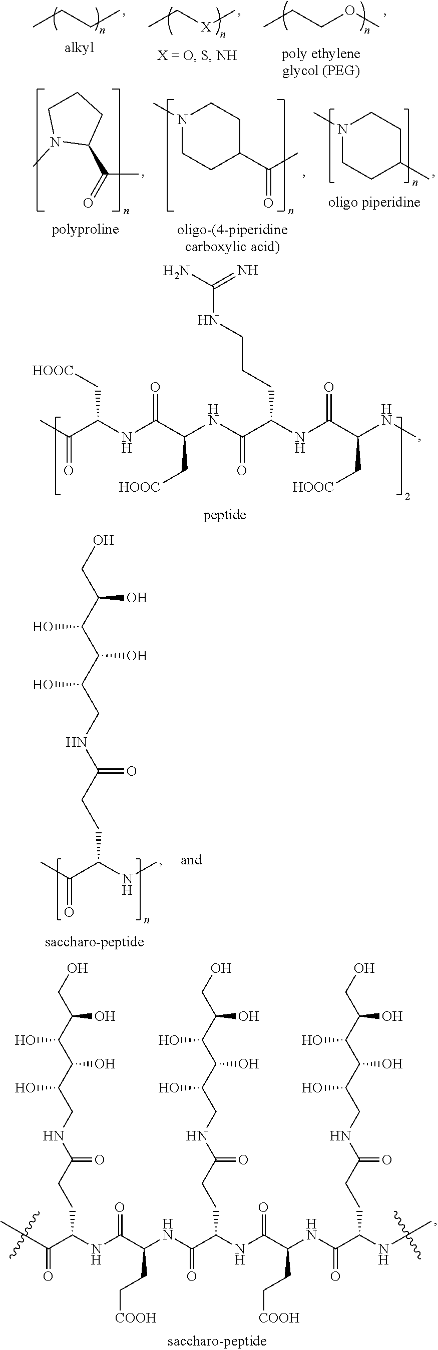

wherein n is an integer from 0 to 200.

In certain embodiments, the small molecule ligand of the one or more adaptor compounds, or pharmaceutically acceptable salts thereof, comprises a structure having the formula:

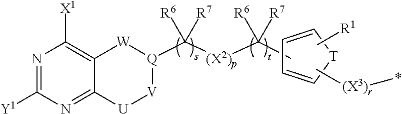

wherein:
X¹ and Y¹ are each independently selected from the group consisting of a halo, R², OR², SR³, and NR⁴R⁵;
U, V, and W represent divalent moieties each independently selected from the group consisting of —(R^{6a})C═, —N═, —(R^{6a})C(R^{7a})—, and —N(R^{4a})—;
Q is selected from the group consisting of C and CH;
T is selected from the group consisting of S, O, N, and —C═C—;
X² and X³ are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C≡CH)—, —N(CH₂C≡CH)—, C₁-C₁₂ alkylene, and C₁-C₁₂ alkyeneoxy, where Z is oxygen or sulfur;
R¹ is selected-from the group consisting of hydrogen, halo, C₁-C₁₂ alkyl, and C₁-C₁₂ alkoxy;
R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;
R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group;
R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;
p, r, s, and t are each independently either 0 or 1; and
* represents a covalent bond, if the one or more adaptor compound, or pharmaceutically acceptable salt thereof, comprises a chemical moiety.

In at least one embodiment, the one or more adaptor compounds, or pharmaceutically acceptable salts thereof comprises at least: a first set of adaptor compounds or pharmaceutically acceptable salts thereof, each adaptor compound or pharmaceutically acceptable salt thereof of the first set comprising a first small molecule ligand linked to the first targeting moiety; and a second set of adaptor compounds or pharmaceutically acceptable salts thereof, each adaptor compound or pharmaceutically acceptable salt thereof of the second set comprising a second small molecule ligand linked to the first targeting moiety; wherein the first small molecule ligand is specific to a receptor overexpressed on a first type of cancer cell and the second small molecule ligand is specific to a receptor overexpressed on a second type of cancer cell.

In certain embodiments, the CAR can have a recognition region comprising a scFv region of an antibody that binds to the first targeting moiety and/or the second targeting moiety with high affinity (e.g., in the sub-nanomolar range). For example, and without limitation, the scFv region can be a scFv region of an anti-FITC antibody. In this manner, when administered, binding the recognition region of the CAR-T cell to the second targeting moiety internalizes the activity modifying compound linked to the second targeting moiety into the CAR-T cell.

The CAR can further comprise a co-stimulation domain and/or an activation signaling domain. In certain embodiments, the co-stimulation domain is selected from a group consisting of: CD28, CD137 (4-1BB), CD134 (OX40), and CD278 (ICOS). In certain embodiments, the activation signaling domain is a T cell CD3ζ chain or an Fc receptor γ.

A method for treating a subject having received CAR-T cell therapy (e.g., by modifying the CAR-T cell activity) is also provided using the disclosed compositions, cells, vectors, and compounds. In some embodiments, the method of treating cancer and/or killing cancer cells in a subject comprises: administering to the subject one or more adaptor compounds, or pharmaceutically acceptable salts thereof, each adaptor compound or pharmaceutically acceptable salt thereof comprising a small molecule ligand linked to a first targeting moiety, wherein, prior to the administering step, the subject has received at least a dose of T cells expressing a CAR that recognizes and binds to the first targeting moiety. In some embodiments, the method further comprises the step of administering to the subject an activity modifying compound linked to a second targeting moiety, wherein the CAR recognizes and binds to the second targeting moiety.

Also provided is a combination for modifying T cell activity (which, for example, can result in treating a cancer). The combination comprises one or more adaptor compounds, or pharmaceutically acceptable salts thereof, wherein each adaptor compound, or pharmaceutically acceptable salt thereof, comprises a small molecule ligand linked to the first targeting moiety; and an activity modifying compound, the activity modifying compound comprising: a rejuvenating compound, or a pharmaceutically acceptable salt thereof, or an immunosuppressive compound, or a pharmaceutically acceptable salt thereof. The one or more adaptor compounds (or pharmaceutically acceptable salts thereof) and the activity modifying compound can be any of the compounds described herein and include, without limitation, a first linker and a second linker, respectively.

Other combinations for modifying T cell activity in a subject with cancer and/or treating cancer comprise one or more adaptor compounds, or pharmaceutically acceptable salts thereof, wherein each adaptor compound, or pharmaceutically acceptable salt thereof, comprises a small molecule ligand linked to the first targeting moiety; an activity modifying compound, the activity modifying compound comprising: a rejuvenating compound, or a pharmaceutically acceptable salt thereof, or an immunosuppressive compound, or a pharmaceutically acceptable salt thereof; and a composition comprising CAR-T cells, wherein the CAR-T cells comprise a CAR directed to the first targeting moiety, the second targeting moiety or both the first and second targeting moiety. For example, the CAR can have a recognition region that binds to the first targeting moiety and/or the second targeting moiety with high affinity (e.g., in a sub-nanomolar range). The one or more adaptor compounds (or pharmaceutically acceptable salts thereof), the activity modifying compound, and the CAR-T cell composition can be any of the compounds and compositions described herein.

Combinations for modifying T cell activity/treating cancer comprising a vector composition are also provided. In some embodiments, the combination comprises one or more adaptor compounds, or pharmaceutically acceptable salts thereof, wherein each adaptor compound, or pharmaceutically acceptable salt thereof, comprises a small molecule ligand linked to the first targeting moiety; an activity modifying compound, the activity modifying compound comprising: a rejuvenating compound, or a pharmaceutically acceptable salt thereof, or an immunosuppressive compound, or a pharmaceutically acceptable salt thereof; and a vector composition comprising a promoter operatively linked to a nucleic acid sequence encoding a CAR. The one or more adaptor compounds (or pharmaceutically acceptable salts thereof), the activity modifying compound, the vector composition, and the CAR can be any of the compounds, compositions, or CARs (as appropriate) described herein and include, without limitation, a first linker and a second linker, respectively. The vector composition can comprise a lentiviral vector. For example, and without limitation, the vector composition can comprise lentiviral particles comprising the nucleic acid vector. In at least one embodiment, the vector composition comprises a therapeutically effective amount of the disclosed viral particles.

Compounds for rejuvenating CAR-T cells are also provided. A compound for rejuvenating CAR-T cells comprises a rejuvenating compound, or a pharmaceutically acceptable salt thereof, selected from a group comprising a TLR agonist, a STING agonist, a NLR, an ALR agonist, a kinase inhibitor targeting kinase, a RLR, a RAGE, a phosphatase inhibitor, and any other pattern recognition receptor that is located in the endosome or cytoplasm of the targeted CAR-T cell.

In certain embodiments, the compound for rejuvenating CAR-T cells, or the pharmaceutically acceptable salt thereof, has a structure of the following formula:

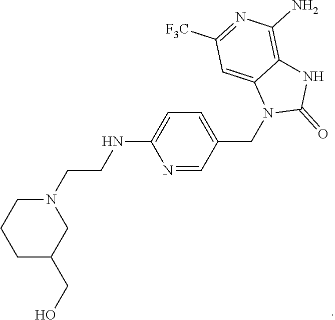 [SMILES: Nc1nc(C(F)(F)F)cc2c1[nH]c(=O)n2Cc1ccc(NCCN2CCCC(CO)C2)nc1]

In certain embodiments, the compound for rejuvenating CAR-T cells, or the pharmaceutically acceptable salt thereof, has a structure of the formula:

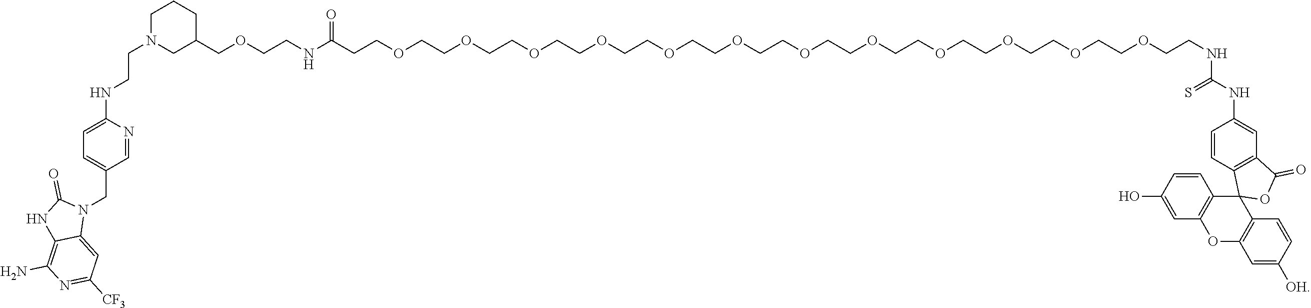 [SMILES: Nc1nc(C(F)(F)F)cc2c1[nH]c(=O)n2Cc1ccc(NCCN2CCCC(COCCNC(=O)CCOCCOCCOCCOCCOCCOCCOCCOCCOCCOCCOCCOCCNC(=S)Nc3ccc4c(c3)C(=O)OC43c4ccc(O)cc4Oc4cc(O)ccc43)C2)nc1]

In certain embodiments, the compound for rejuvenating CAR-T cells, or the

pharmaceutically acceptable salt thereof, has a structure of one of the following formulae:

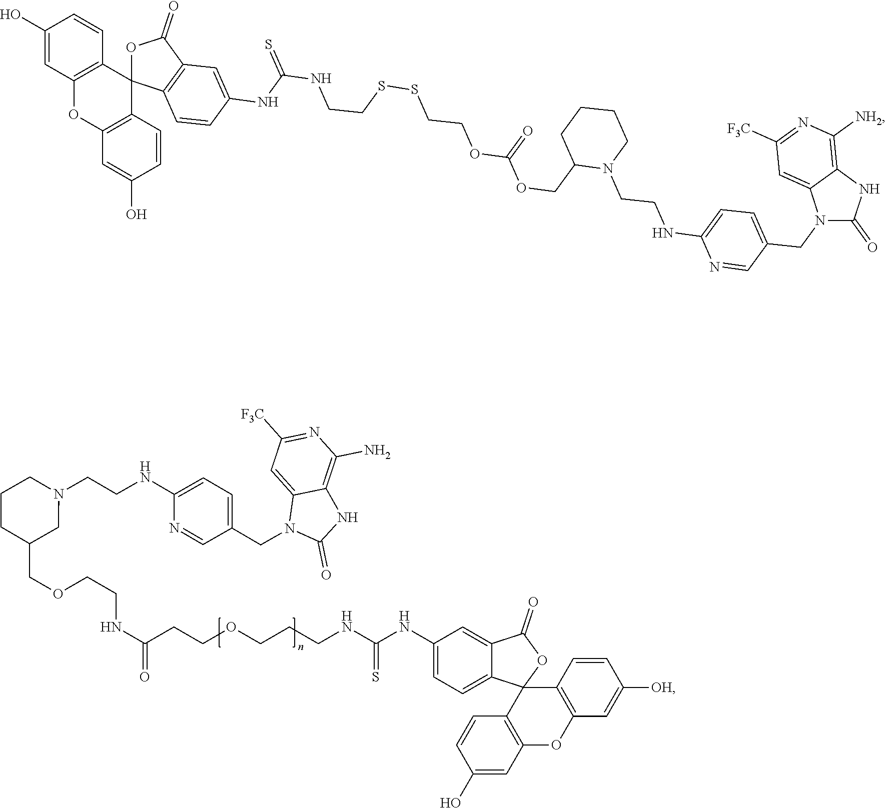

wherein n=0 to 200, or

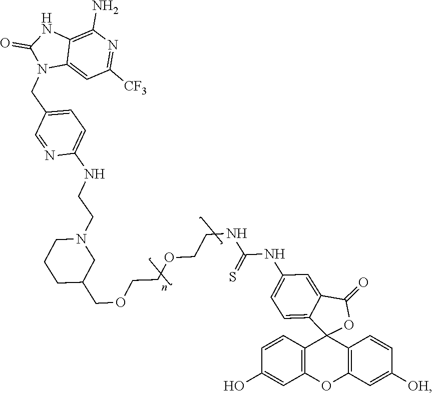

wherein n=0 to 50.

In certain embodiments, the compound for rejuvenating CAR-T cells, or the pharmaceutically acceptable salt thereof, has the following formula:

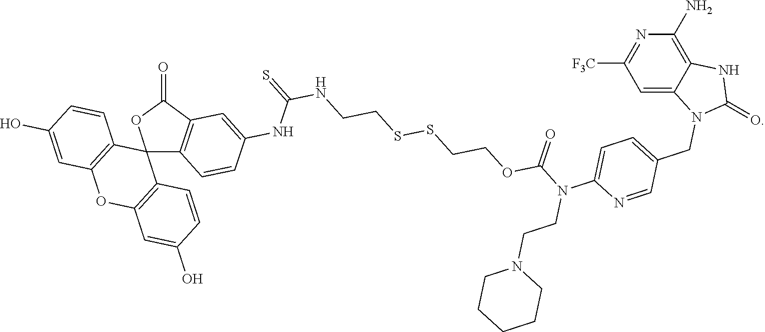 [SMILES: Nc1nc(C(F)(F)F)cc2c1[nH]c(=O)n2Cc1ccc(N(CCN2CCCCC2)C(=O)OCCSSCCNC(=S)Nc2ccc3c(c2)C(=O)OC32c3ccc(O)cc3Oc3cc(O)ccc32)nc1]

### BRIEF DESCRIPTION OF THE DRAWINGS

The embodiments and other features, advantages, and aspects, and the matter of attaining them, will become apparent in light of the following detailed description of various examplary embodiments. The detailed description will be better understood when taken in conjunction with the accompanying drawings.

FIG. 1 shows a general diagram of constructs that can be used for CAR-T cell transduction.

FIG. 2A shows an illustration of the in vitro exhaustion model.

FIG. 2B shows that anti-FITC CAR-T cells became exhausted after being stimulated twice with fresh MDA-MB-231 cells in vitro as shown by the decreased killing effect and FIG. 2C shows the corresponding increased expression of T cell exhaustion markers (PD-1+Tim3+LAG3+).

FIG. 3 shows the structure of TLR7 agonist-FITC conjugates.

FIG. 3B shows the rejuvenation effect of the TLR7 agonist-FITC conjugates on exhausted anti-FITC CAR-T cells in vitro in the exhaustion model, shown as the increased killing effect; FITC-AF647 has a K_{d}=8.03 nM towards anti-FITC CAR-T cells;

FIG. 4A shows the rejuvenation effect of the FITC-TLR7 agonist conjugates in a KB xenograft model, shown as decreased tumor size.

FIG. 4B and FIG. 4C show the rejuvenation effect of the FITC-TLR7 agonist conjugates in a KB xenograft model, shown as decreased expression of exhaustion markers (PD-1⁺Tim3⁺).

FIG. 4D shows the change in CAR T cell population.

FIG. 4E shows the change in mice body weight.

Wherever feasible and convenient, like reference numerals are used in the figures and the description to refer to the same or like parts or steps. The drawings are in a simplified form and not to precise scale. While the present disclosure is susceptible to various modifications and alternative forms, examplary embodiments thereof are shown in the drawings and are described in detail.

### BRIEF DESCRIPTION OF THE SEQUENCE LISTINGS

SEQ ID NO: 1 is an examplary nucleic acid sequence for encoding a chimeric antigen receptor (CAR);

SEQ ID NO: 2 is an exemplary CAR amino acid sequence encoded by SEQ ID NO: 1 or 3; and

SEQ ID NO: 3 is an examplary nucleic acid sequence for encoding a CAR.

The above-described sequences are set forth in the Sequence Listing Section below and also provided in computer readable form encoded in a file filed herewith and herein incorporated by reference. The information recorded in computer readable form is identical to the written Sequence Listings provided herein, pursuant to 37 C.F.R. § 1.821(f).

### DETAILED DESCRIPTION

The present disclosure relates to the preparation and use of compounds and compositions that reduce the propensity for off-target toxicity following administration of T cells expressing a chimeric antigen receptor (CAR-T cells) to a subject as compared to conventional CAR-T cell therapies. The term “off-target toxicity” means organ or tissue damage or a reduction in the subject's weight that is not desirable to the physician treating the subject, or any other effect on the subject that are potential adverse indicators to the treating physician, for example, B cell aplasia, a fever, a drop in blood pressure, or pulmonary edema. The terms “treat,” “treating,” “treated,” or “treatment” (with respect to a disease or condition) is an approach for obtaining beneficial or desired results including and preferably clinical results and includes, but is not limited to, one or more of the following: improving a condition associated with a disease, curing a disease, lessening severity of a disease, delaying progression of a disease, alleviating one or more symptoms associated with a disease, increasing the quality of life of one suffering from a disease, prolonging survival and/or prophylactic or preventative treatment. In reference to cancer, in particular, the terms “treat,” “treating,” “treated,” or “treatment” can additionally mean reducing the size of a tumor, completely or partially removing the tumor (e.g., a complete or partial response), causing stable disease, preventing progression of the cancer (e.g., progression free survival), or any other effect on the cancer that would be considered by a physician to be a therapeutic, prophylactic, or preventative treatment of the cancer.

In various embodiments, the compositions comprise genetically engineered CAR-T cells and at least one adaptor compound. The adaptor compound can have specificity to one or more targeted cancer cells and is adapted to bind with a chimeric antigen receptor (CAR) of the CAR-T cells via a targeting moiety such that, when administered, the adaptor compound forms a bridge between the targeted cancer cell and the CAR-T cell. In at least one embodiment, the CAR of the CAR-T cells is directed to the targeting moiety of the adaptor compound such that it binds therewith with specificity and, thus, reduces off-target toxicity and other interactions. In this manner, the CAR-T cell can bind to the adaptor compound and, when so bound, the adaptor compound can direct the CAR-T cells to the targeted cancer cells for treatment of the cancer.

Various embodiments are formulated to enhance control of CAR-T cell activation in vivo following administration of CAR-T cells to a subject. For example, embodiments of such compositions can comprise genetically engineered CAR-T cells and at least one activity modifying compound (e.g., a rejuvenating compound, an immunosuppressive compound, or a pharmaceutically acceptable salt of either of the foregoing). The CAR-T cell can comprise a CAR directed to a targeting moiety of the activity modifying compound such that when the CAR-T cell and the activity modifying compound are administered to a subject, the CAR-T cell links (e.g., with specificity) to the targeting moiety coupled with the activity modifying compound. In at least one embodiment, the recognition region of the CAR can additionally be exploited to deliver the activity modifying compound into the CAR-T cell. Such embodiments can be used in lieu of, or in conjunction with, the adaptor compounds.

As used herein, “CAR-T cells” refer to a T cell or population thereof that has been modified through molecular biological methods to express a chimeric antigen receptor (CAR) on the T cell surface. The CAR is a polypeptide having a pre-defined binding specificity to a desired target and is operably connected to (e.g., as a fusion, separate chains linked by one or more disulfide bonds, etc.) the intracellular part of a T cell activation domain. By bypassing MHC class I and class II restriction, CAR engineered T cells of both CD8+ and CD4+ subsets can be recruited for redirected target cell recognition.

As is described in further detail below, the CARs comprise a recognition region as is further defined herein. In certain embodiments, a CAR can additionally include an activation signaling domain that, for example, can be derived from a T cell CD3-zeta (CD3ζ) chain, a Fc receptor gamma signaling domain or a Fc receptor γ, or one or more costimulatory domains such as CD28, CD137 (4-1BB), CD278 (ICOS), or CD134 (OX40).

Certain CARs are fusions of binding functionality (e.g., as a single-chain variable fragment (scFv) derived from a monoclonal antibody) to CD3ζ transmembrane and endodomain. Such molecules result in the transmission of a zeta signal in response to recognition by the recognition receptor binding functionality of its target. There are, however, many alternatives. By way of non-limiting example, an antigen recognition domain from native T cell receptor (TCR) alpha and beta single chains can be used as the binding functionality. Alternatively, receptor ectodomains (e.g., CD4 ectodomain) can be employed. All that is required of the binding functionality is that it can bind a given target with high affinity in a specific manner.

Additionally, “binds with specificity,” “binds with high affinity,” or “specifically” or “selectively” binds, when referring to a ligand/receptor, a recognition region/targeting moiety, a nucleic acid/complementary nucleic acid, an antibody/antigen, or other binding pair indicates a binding reaction that is determinative of the presence of the protein in a heterogeneous population of proteins and other biologics. Thus, under designated conditions, a specified ligand or recognition region binds to a particular receptor (e.g., one present on a cancer cell) or targeting moiety, respectively, and does not bind in a significant amount to other proteins present in the sample (e.g., those associated with normal, healthy cells). Specific binding or binding with high affinity can also mean, for example, that the binding compound, ligand, antibody, or binding composition derived from the antigen-binding site of an antibody, of the contemplated method binds to its target with an affinity that is often at least 25% greater, more often at least 50% greater, most often at least 100% (2-fold) greater, normally at least ten times greater, more normally at least 20-times greater, and most normally at least 100-times greater than the affinity with any other binding compound.

In a typical embodiment, a molecule that specifically binds a target will have an affinity that is at least about 10⁶ liters/mol (K_{O}=10^{˜6} M), and preferably at least about 10 liters/mol, as determined, for example, by Scatchard analysis. It is recognized by one of skill in the art that some binding compounds can specifically bind to more than one target, for example an antibody specifically binds to its antigen, to lectins by way of the antibody's oligosaccharide, and/or to an Fc receptor by way of the antibody's Fc region.

The compounds, compositions, and methods will now be described in detail. For the purposes of promoting an understanding of the principles presented herein, reference is made to the embodiments illustrated in the drawings and specific language will be used to describe the same. It will nevertheless be understood that no limitation of scope is intended by the description of these embodiments. On the contrary, this disclosure is intended to cover alternatives, modifications, and equivalents as may be included within the spirit and scope of this application as defined by the appended claims. As previously noted, while this technology may be illustrated and described in one or more preferred embodiments, the compositions, compounds and methods hereof may comprise many different configurations, forms, materials, and accessories.

As noted above, certain compounds comprise at least one adaptor compound (or a pharmaceutically acceptable salt thereof) adapted to form a bridge between a cancer cell and a CAR-T cell, where the CAR-T cell comprises a CAR directed to a targeting moiety of the adaptor compound. Accordingly, the CAR-T cells can bind to the targeting moiety of the adaptor compound and, when so bound, the adaptor compound can direct the CAR-T cells to the cancer for treatment of the cancer.

### Adaptor Compounds

In at least one embodiment, the adaptor compound (or pharmaceutically acceptable salt thereof) is a small molecule ligand linked to a first targeting moiety, via a first linker or otherwise. The small molecule ligand can be any small molecule ligand that binds with specificity to a cancer cell type (i.e., wherein the receptor for such ligand is overexpressed on targeted cancer cells as compared to normal tissues or other non-targeted types of cancer). As used herein, a “ligand” is a molecule, ion, or atom that is attached to the central atom or ion (e.g., a drug) of a compound. “Ligand” also encompasses a binding agent that is not an agonist or antagonist and has no agonist or antagonist properties. Further, the terms “overexpressed,” “overexpression,” and their formatives (when used in connection with receptor expression and the like) have the meaning ascribed thereto by one of ordinary skill in the relevant arts, which includes (without limitation) the increased presence of a particular receptor on a target cell (e.g., a tumor cell) as compared to normal tissues or other non-targeted types of cells.

In at least one embodiment, the small molecule ligand of the adaptor compound is a folate, a carbonic anhydrase IX (CAIX) ligand, a 2-[3-(1,3-dicarboxypropyl)ureido]pentanedioic acid (DUPA) ligand, a neurokinin 1 receptor (NK-1R) ligand, a ligand of gamma glutamyl transpeptidase, a natural killer group 2D receptor (NKG2D) ligand, or a cholecystokinin B receptor (CCKBR or CCK2) ligand. Each of the aforementioned is a small molecule ligand that binds specifically to a receptor that is overexpressed on a certain cancer cell type (i.e., the receptor for each of these ligands is overexpressed on cancers compared to expression of such receptor on normal tissues or, potentially, in diseased tissue not experiencing the targeted cancer type). Indeed, receptors for the CAIX ligand are found, for example, on renal, ovarian, vulvar, and breast cancer; receptors for the NK-1R ligand are found, for example, on cancers of the colon and pancreas; DUPA ligand is a ligand bound by PSMA-positive human prostate cancer cells and other cancer cell types; receptors for the NKG2D ligand are found, for example, on cancers of the lung, colon, kidney, prostate, and on T and B cell lymphomas; receptors for the CCKBR ligand are found on cancers of the thyroid, lung, pancreas, ovary, brain, stomach, gastrointestinal stroma, and colon, among others; and the transpeptidase is overexpressed, for example, in ovarian cancer, colon cancer, liver cancer, astrocytic gliomas, melanomas, and leukemias.

In other embodiments, the adaptor compound, or the pharmaceutically acceptable salt thereof, is not an antibody, and does not comprise a fragment of an antibody. In yet another embodiment, the first targeting moiety does not comprise a peptide epitope.

In one embodiment, the small molecule ligand can have a mass of less than about 10,000 Daltons, less than about 9,000 Daltons, less than about 8,000 Daltons, less than about 7,000 Daltons, less than about 6,000 Daltons, less than about 5,000 Daltons, less than about 4,500 Daltons, less than about 4,000 Daltons, less than about 3,500 Daltons, less than about 3,000 Daltons, less than about 2,500 Daltons, less than about 2,000 Daltons, less than about 1,500 Daltons, less than about 1,000 Daltons, or less than about 500 Daltons. In another embodiment, the small molecule ligand can have a mass of about 1 to about 10,000 Daltons, about 1 to about 9,000 Daltons, about 1 to about 8,000 Daltons, about 1 to about 7,000 Daltons, about 1 to about 6,000 Daltons, about 1 to about 5,000 Daltons, about 1 to about 4,500 Daltons, about 1 to about 4,000 Daltons, about 1 to about 3,500 Daltons, about 1 to about 3,000 Daltons, about 1 to about 2,500 Daltons, about 1 to about 2,000 Daltons, about 1 to about 1,500 Daltons, about 1 to about 1,000 Daltons, or about 1 to about 500 Daltons. In other embodiments, these masses can apply to the targeting moieties as well.

In one embodiment, a DUPA derivative can be the ligand of the small molecule ligand linked to the first targeting moiety in the adaptor compound, or the pharmaceutically salt thereof. Such DUPA derivatives are described in International Publication No. WO 2015/057852, which is incorporated herein by reference in its entirety for its teachings regarding same.

In at least one embodiment, the small molecule ligand is a folate. “Folate” can be folic acid, a folic acid analog, or another folate receptor-binding molecule, including for example, analogs and derivatives of folic acid such as, without limitation, folinic acid (e.g., leucovorin), pteroylpolyglutamic acid, pteroyl-D-glutamic acid, and folate receptor-binding pterdines such as tetrahydropterins, dihydrofolates, tetrahydrofolates, and their deaza and dideaza analogs.

An “analog” or “derivative” with reference to a peptide, polypeptide or protein refers to another peptide, polypeptide or protein that possesses a similar or identical function as the original peptide, polypeptide or protein, but does not necessarily comprise a similar or identical amino acid sequence or structure of the original peptide, polypeptide or protein. An analog preferably satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the original amino acid sequence; (b) a proteinaceous agent encoded by a nucleotide sequence that hybridizes under stringent conditions to a nucleotide sequence encoding the original amino acid sequence; or (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding the original amino acid sequence.

The terms “deaza” and “dideaza” analogs refer to the art-recognized analogs having a carbon atom substituted for one or two nitrogen atoms in the naturally occurring folic acid structure, or analog or derivative thereof. For example, the deaza analogs can include the 1-deaza, 3-deaza, 5-deaza, 8-deaza, and 10-deaza analogs of folate, folinic acid, pteropolyglutamic acid, and folate receptor-binding pteridines such as tetrahydropterins, dihydrofolates, and tetrahydrofolates. The dideaza analogs include, for example, 1,5-dideaza, 5,10-dideaza, 8,10-dideaza, and 5,8-dideaza analogs. The foregoing folic acid analogs are conventionally termed “folates,” reflecting their capacity to bind to folate receptors. Other folate receptor-binding analogs include aminopterin, amethopterin (methotrexate), N10-methylfolate, 2-deamino-hydroxyfolate, deaza analogs such as 1-deazamethopterin or 3-deazamethopterin, and 3′,5′-dichloro-4-amino-4-deoxy-N¹⁰-methylpteroylglutamic acid (dichloromethotrexate).

The foregoing analogs and/or derivatives are also termed “a folate,” “the folate,” or “folates” reflecting their ability to bind to folate-receptors. Such molecules, when conjugated with exogenous molecules, are effective to enhance transmembrane transport, such as via folate-mediated endocytosis. The foregoing can be used in the folate receptor-binding ligands described herein.

In another embodiment, the small molecule ligand of the adaptor compound hereof can have the formula:

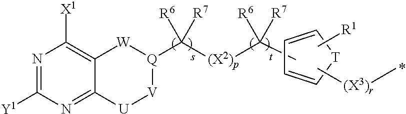

wherein:
X¹ and Y¹ are each independently selected from the group consisting of a halo, R², OR², SR³, and NR⁴R⁵;
U, V, and W represent divalent moieties each independently selected from the group consisting of —(R^{6a})C═, —N═, —(R^{6a})C(R^{7a})—, and —N(R^{4a})—;
Q is selected from the group consisting of C and CH;
T is selected from the group consisting of S, O, N, and —C═C—;
X² and X³ are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C≡CH)—, —N(CH₂C≡CH)—, C₁-C₁₂ alkylene, and C₁-C₁₂ alkyeneoxy, where Z is oxygen or sulfur;
R¹ is selected-from the group consisting of hydrogen, halo, C₁-C₁₂ alkyl, and C₁-C₁₂ alkoxy;
R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;
R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group;
R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;
p, r, s, and t are each independently either 0 or 1; and
* represents an optional covalent bond to the rest of the conjugate, if any additional chemical moieties are part of or coupled with the folate.

In one aspect, the cancer may overexpress (as compared to a normal tissue or cell or another type of cancer) a receptor for the small molecule ligand. In one illustrative embodiment, the adaptor compound comprises fluorescein isothiocyanate (FITC) linked to the small molecule ligand. In another aspect, for example, cytotoxic T cells, or another type of T cell, can be transformed to express a CAR that comprises anti-FITC scFv. In this aspect, the CAR can target FITC decorating the cancer as a result of binding of the small molecule ligand in the adaptor compound to the cancer. Thus, toxicity to normal, non-target cells can be avoided. In this embodiment, when the anti-FITC CAR-expressing T cells bind FITC, the CAR-T cells are activated and the cancer is treated.

The adaptor compound is linked to a first targeting moiety (directly or, for example, via a first linker). The first targeting moiety of the adaptor compound is configured to bind to a recognition region of a genetically engineered CAR expressed by a population of CAR-T cells. For example, and without limitation, the first targeting moiety can comprise 2,4-dinitrophenol (DNP), 2,4,6-trinitrophenol (TNP), biotin, digoxigenin, fluorescein, FITC, NHS-fluorescein, pentafluorophenyl ester, tetrafluorophenyl ester, a knottin, a centyrin, a DARPin, an affibody, an affilin, an anticalin, an atrimer, an avimer, a bicicyclic peptide, an FN3 scaffold, a cys-knot, a fynomer, a Kunitz domain, or an Obody. Various embodiments of available targeting moieties are described below and any of these embodiments can be employed with the first targeting moiety of the adaptor compound.

In at least one embodiment, the small molecule ligand is linked to the first targeting moiety via a first linker and comprises the following structure:

B—L—T,

wherein B represents the small molecule ligand, L represents the first linker, and T represents the first targeting moiety. Various embodiments of available linkers are described below and any of these embodiments can be employed with the first linker of the adaptor compound.

When administered to a subject, the small molecule ligand linked to the first targeting moiety (e.g., by a first linker) acts as a bridge between the target cancer cell and the CAR-T cell and directs the CAR-T cells to the cancer for treatment thereof. In various embodiments, the bridge (i.e., the adaptor compound, or a pharmaceutically acceptable salt thereof) between the cancer and the CAR-T cells can be any of the adaptor compounds described herein or shown in the Examples.

In at least one embodiment, the adaptor compound (or pharmaceutically acceptable salt thereof) is a small organic molecule so clearance from the bloodstream following administration can be rapidly achieved (i.e., in about 20 minutes or less).

The CAR-T cell population with which the at least one adaptor compound is to be administered comprises a CAR having a recognition region (e.g., a scFv of an antibody, a Fab fragment, a variable region (Fv), a Fc region, a (Fab′)2 fragment, or the like) directed to the first targeting moiety. In certain embodiments, for example, where the first targeting moiety of the adaptor compound is a FITC antibody, the recognition region of the CAR is a scFv region of an anti-FITC antibody.

In at least one embodiment, the recognition region of the CAR binds to the first targeting moiety with high affinity such as, for example, in the sub-nanomolar range. Accordingly, when administered, the CAR-T cells will bind to the targeting moiety of the adaptor compound (or pharmaceutically acceptable salt thereof) and the CAR-T cell response can be targeted to only those cancer cells expressing a receptor for the small molecule ligand portion of the ‘bridge,’ thereby reducing off-target toxicity to normal tissues. Indeed, the small molecule ligand can direct any CAR-T cell linked thereto to a target cell with specificity.

Use of the at least one adaptor compound (or pharmaceutically acceptable salt thereof) provides ‘universality’ because a single type of CAR-T cell, with a single type of recognition region, can be used with multiple adaptor compounds to eradicate multiple tumor types. Illustratively, the targeting moiety of each adaptor compound as recognized by the CAR-T cell can remain constant so that one type of CAR-T cell construct can be used, while the small molecule ligand that binds to the cancer can be altered between adaptor compounds to allow for targeting of a wide variety of cancers. For example, and without imitation, a first set and a second set of adaptor compounds may be administered to a subject, wherein the first set comprises a first small molecule ligand linked to the first targeting moiety and the second set comprises a second small molecule ligand linked to the first targeting moiety. In such embodiment, the first small molecule ligand is specific to a receptor overexpressed on a first type of cancer cell (as compared to a baseline expression of such receptor on a healthy tissue or a different type of cancer cell, collectively, a “non-targeted cell”) and the second small molecule ligand is specific to a receptor overexpressed on a second type of cancer cell (as compared to a baseline expression of such receptor on a non-targeted cell). While both sets of adaptor compound (or pharmaceutically acceptable salts thereof) are linked to the same targeting moiety (i.e., the first targeting moiety) such that the CAR is directed to bind therewith, it will be appreciated that the different small molecule ligands are targeted to, and can be used to treat, different cancer cell types. Accordingly, the adaptor compounds make the CAR-T cells ‘universal’ CAR-T cells for killing tumors that express different antigens because the different small molecule ligands in the adaptor compounds bind to different tumor types, but only one type of CAR-T cell with one type of recognition region need be used.

In certain embodiments, the small molecule ligand linked to the first targeting moiety by a first linker (i.e., the ‘bridge’ or adaptor compound) can have any of the following structures:

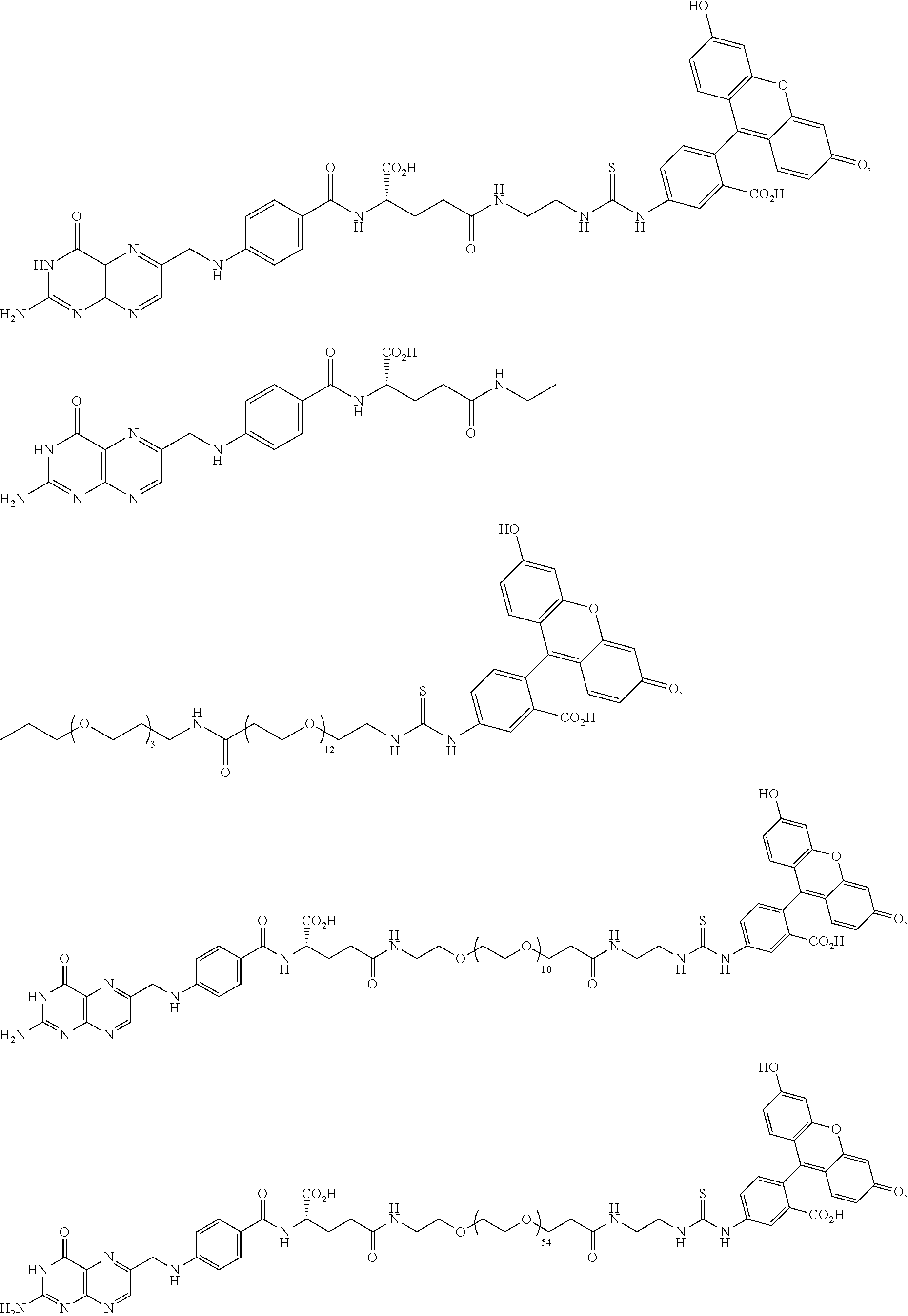

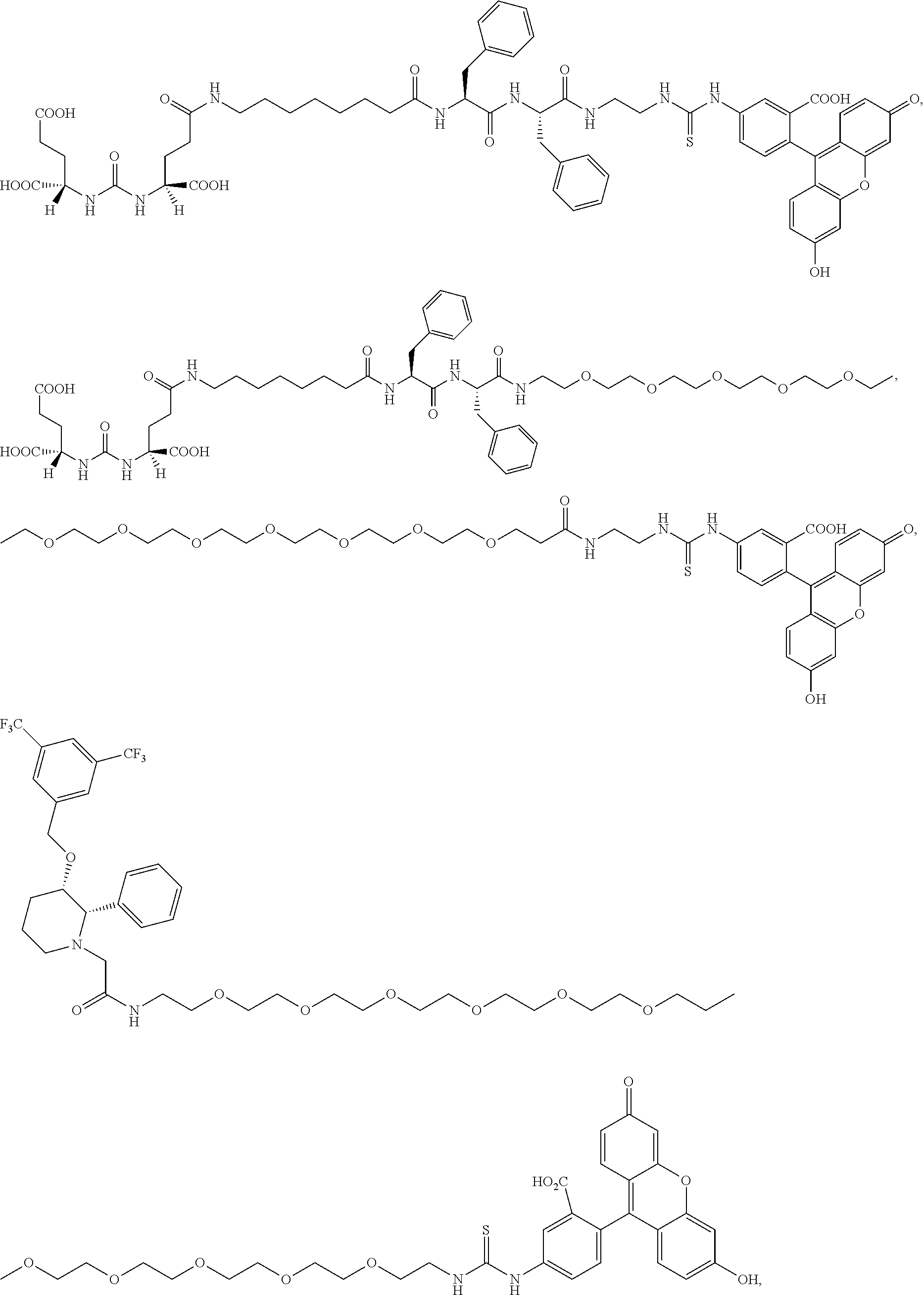

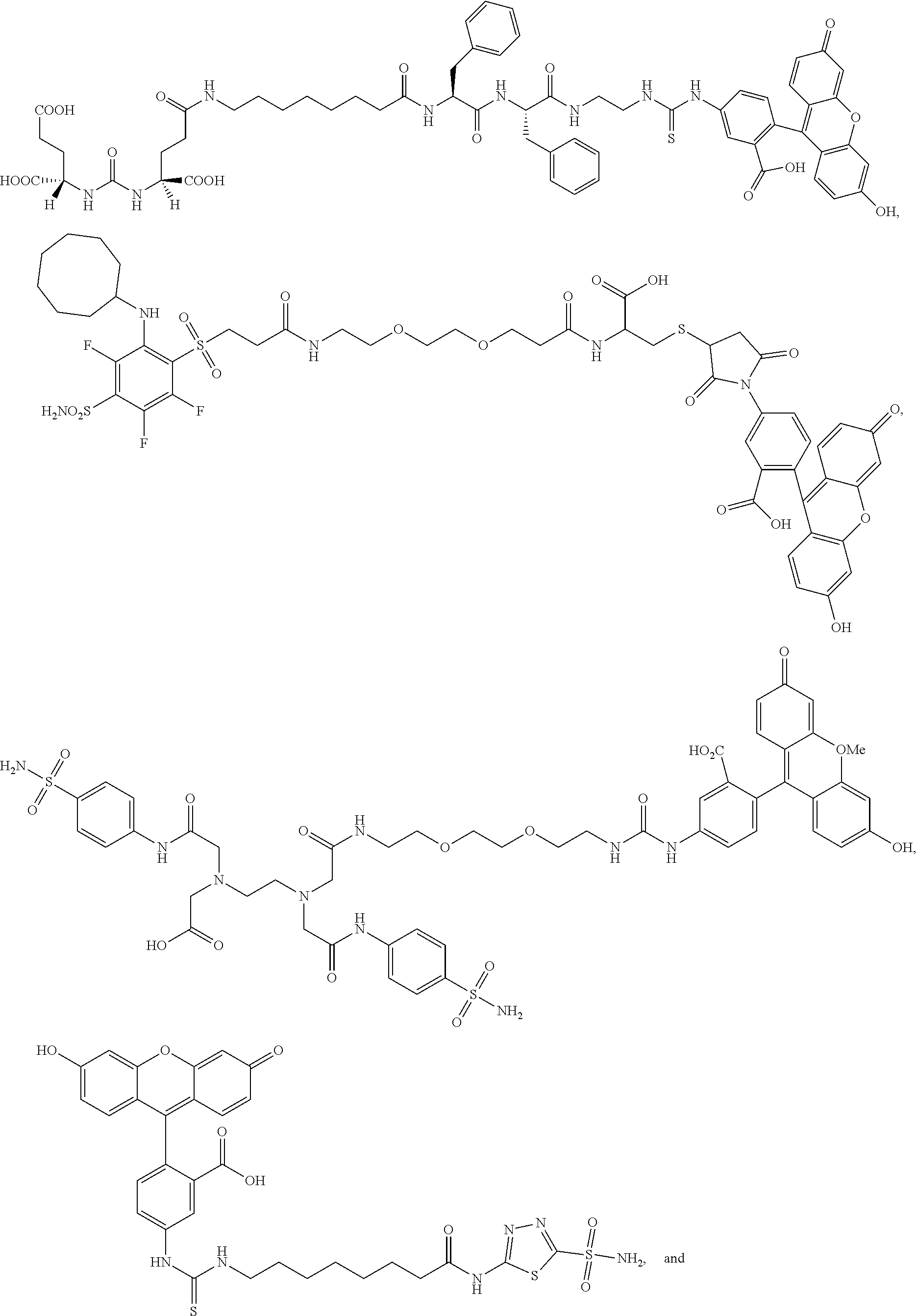

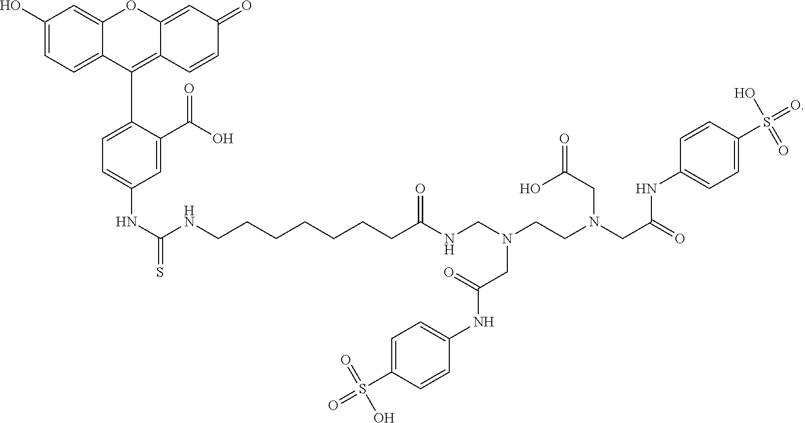 [SMILES: O=C(O)CN(CCN(CNC(=O)CCCCCCCNC(=S)Nc1ccc(-c2c3ccc(=O)cc-3oc3cc(O)ccc23)c(C(=O)O)c1)CC(=O)Nc1ccc(S(=O)(=O)O)cc1)CC(=O)Nc1ccc(S(=O)(=O)O)cc1]

### Activity Modifying Compounds

A common problem faced by conventional CAR-T cell therapies is that the CAR-T cells may become dysfunctional or “exhausted” such that reduced proliferation results upon chronic exposure to tumor antigens or immunosuppressive factors (e.g., myeloid-derived suppressor cells (MDSCs), tumor-associated macrophages (TAMs), regulatory T cells (Tregs), and inhibitory cytokines) in the tumor microenvironment. T cell exhaustion is defined by poor effector function, sustained expression of inhibitory receptors and a transcriptional state distinct from that of functional effector or memory T cells. Exhaustion prevents optimal control of tumors and reduced T cell exhaustion can result in improved clinical responses.

In contrast, CAR-T cells can also become overactive resulting in unwanted side effects of conventional CAR-T cell therapies, such as cytokine release syndrome (CRS) and other toxicities, which can be fatal to the patient. These conditions can result from high-levels of CAR-T cell expansion and immune activation, which ultimately causes lysis of both normal and tumor cells and the release of several cytokines such as interferon gamma (IFN-γ) and tumor-necrosis factor alpha (TNFα). The combination of these signals also triggers the activation of monocytes and macrophages with enhanced tumoricidal capacity and that secrete high levels of pro-inflammatory cytokines (e.g., interleukin 6 (IL-6), interleukin 1 (IL-1), and interleukin 10 (IL-10)) and other mediators such as inducible nitric oxide synthase (iNOS), all of which can promote the progression of CRS and other related toxicities.

To address the unwanted side effects of CAR-T cell therapies, certain compounds and compositions hereof comprise an activity modifying compound or a pharmaceutically acceptable salt thereof for targeting and modifying activity of CAR-T cells when administered in conjunction therewith. For example, the activity modifying compound comprises a rejuvenating compound for rejuvenating CAR-T cells, an immunosuppressive compound for reducing activity of CAR-T cells, or a pharmaceutically acceptable salt of either of the foregoing.

Additionally, the recognition region of a CAR (e.g., a scFv fragment) of the CAR-T cells can be modified to exploit endocytosis and facilitate the internalization of the activity modifying compounds into the CAR-T cell. In one embodiment, the immunosuppressive compound, or the pharmaceutically acceptable salt thereof, can be internalized into the CAR-T cells in this manner such that the immunosuppressive compound is concentrated in the CAR-T cell, and, as a result, CAR-T cell activation is reduced, thus leading to control of adverse side effects of excessive CAR-T cell activation such as CRS. In this manner, an immunosuppressive compound, or a pharmaceutically acceptable salt thereof, can be used with the CAR-T cell compositions to target CAR-T cells and can reduce CAR-T cell activation.

Similarly, a rejuvenating compound, or a pharmaceutically acceptable salt thereof, can be internalized into the CAR-T cells via the recognition region of the CAR. By concentrating a rejuvenating compound (or, where applicable, an active component thereof) within the CAR-T cell, exhausted or dysfunctional CAR-T cells can be rejuvenated to functional and tumor-killing CAR-T cells, leading to renewed eradication of tumors. Accordingly, a rejuvenating compound, or a pharmaceutically acceptable salt thereof, can be used with the CAR-T cell compositions to target CAR-T cells and can reverse exhaustion or dysfunction of the CAR-T cells induced by the tumor microenvironment.

The activity modifying compound (or a pharmaceutically acceptable salt thereof) can be linked to a second targeting moiety and at least a portion of the recognition region of a CAR of the CAR-T cells is directed to the second targeted moiety. Various embodiments of available targeting moieties are described below and any of these embodiments may be employed with the second targeting moiety of the activity modifying compound. Accordingly, the CAR-T cells can bind with high affinity to the second targeting moiety of the activity modifying compound.

In at least one embodiment, the activity modifying compound is linked to the second targeting moiety via a second linker. The first linker in the adaptor compound and the second linker in the activity modifying compound can have the same structures or different structures. In one embodiment, the second linker in the activity modifying compound, or the pharmaceutically acceptable salt thereof, can be a releasable linker or a non-releasable linker, and the first linker in the adaptor compound, or the pharmaceutically acceptable salt thereof, can be a non-releasable linker. Various embodiments of available linkers are described below and any of these embodiments may be employed with the second linker of the activity modifying compound.

### Rejuvenating Compounds

“Rejuvenating CAR-T cells” means activating CAR-T cells, increasing proliferation of CAR-T cells, blocking the inhibitory signaling of exhausted or dysfunctional CAR-T cells, re-activating CAR-T cells through an antigen-independent pathway, or otherwise increasing the function of CAR-T cells. Where the activity modifying compound comprises a rejuvenating compound (or a pharmaceutically acceptable salt thereof), embodiments of the present compounds can be particularly useful in preventing or reversing T cell exhaustion or dysfunction, reduced proliferation, and like conditions induced by the tumor microenvironment.

The rejuvenating compound or pharmaceutically acceptable salt thereof can comprise any drug or other compound that can rejuvenate T cells such as, for example, a toll-like receptor (TLR) agonist (e.g., agonists of TLR1, TLR2, TLR3, TLR4, TLR7, TLR8, TLR7/8, TLR9, etc.), a stimulator of interferon genes (STING) agonist, a Nod-like receptor (NLR) stimulant, an absent in melanoma 2 (AIM2)-like receptor (ALR) agonist, a kinase inhibitor targeting kinases such as GSK-3beta, PI3K, etc., and/or a phosphatase inhibitor.

In certain embodiments, the rejuvenating compound or pharmaceutically acceptable salt thereof comprises a retinoic acid-inducible gene-I (RIG-I)-like receptor (RLR), a receptor for advanced glycation end products (RAGE), or any other pattern recognition receptor that is located in the endosome or cytoplasm of a cell.

As used herein, “pattern recognition receptors” means any immune receptors that are expressed on the membranes of leukocytes and are capable of binding specific ligands that activate the receptor and ultimately lead to an innate immune response. Further, TLRs are a class of proteins that play a key role in the innate immune system and are an example of pattern recognition receptors. TLRs are typically single, membrane-spanning receptors that recognize structurally conserved molecules derived from microbes and are typically expressed on the membranes of leukocytes including dendritic cells, macrophages, natural killer (NK) cells, cells of adaptive immunity (i.e. T and B lymphocytes) and non-immune cells (epithelial and endothelial cells and fibroblasts).

In at least one embodiment, the rejuvenating compound, or the pharmaceutically acceptable salt thereof, has a structure of one of the following formulae:

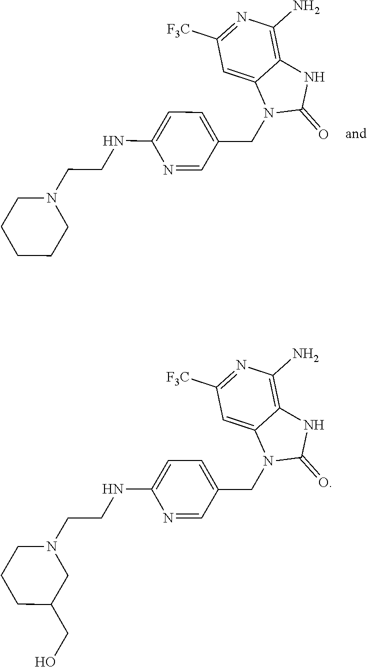 [SMILES: Nc1nc(C(F)(F)F)cc2c1[nH]c(=O)n2Cc1ccc(NCCN2CCCCC2)nc1 ; Nc1nc(C(F)(F)F)cc2c1[nH]c(=O)n2Cc1ccc(NCCN2CCCC(CO)C2)nc1]

In some embodiments, the rejuvenating compound, or pharmaceutically acceptable

salt thereof, has a structure of one of the following formulae:

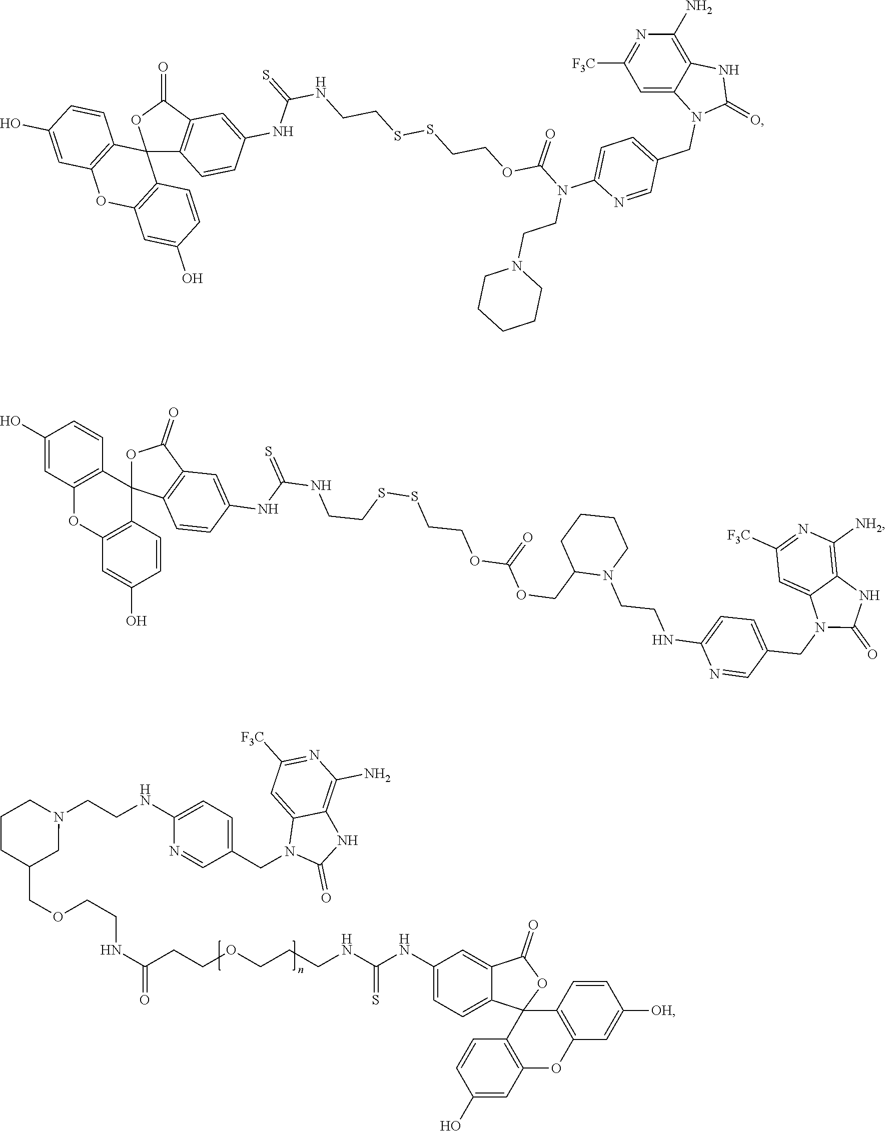

wherein n=0 to 200, and

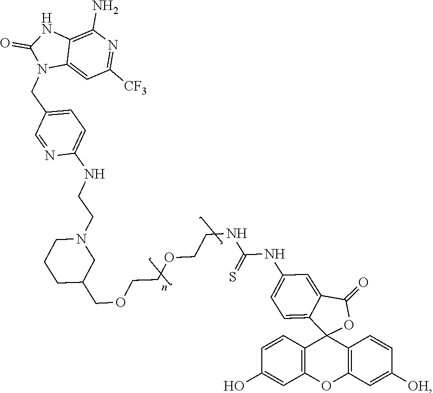

wherein n=0 to 50.

### Immunosuppressive Compounds

In another embodiment, the activity modifying compound comprises an immunosuppressive compound, or a pharmaceutically acceptable salt thereof, which can target and reduce the activity of CAR-T cells. The “reducing activity” of CAR-T cells means suppressing any activity of CAR-T cells, killing CAR-T cells, reducing the number of CAR-T cells, or preventing or reducing proliferation of CAR-T cells. Such compositions can be useful in treating or preventing CRS or other toxicities.

The immunosuppressive compound or pharmaceutically acceptable salt thereof can comprise any drug or other compound capable of reducing the activity of T cells such as, for example, tacrolimus, sirolimus, cyclosporine and/or other immunosuppressive compounds.

### Pharmaceutically Acceptable Salts

A “pharmaceutically acceptable salt” of a small molecule ligand linked to the first targeting moiety (i.e., the adaptor compound) or of an activity modifying compound (i.e., the rejuvenating compound or the immunosuppressive compound) linked to a second targeting moiety are contemplated. The term “pharmaceutically acceptable salt” refers to those salts whose counter ions may be used in pharmaceuticals. In various embodiments, such salts include, but are not limited to 1) acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or 2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, trimethamine, N-methylglucamine, and the like. Pharmaceutically acceptable salts are well-known to those skilled in the art, and any such pharmaceutically acceptable salt is contemplated in connection with the embodiments described herein.

In various embodiments, suitable acid addition salts are formed from acids which form non-toxic salts. Illustrative examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

In various embodiments, suitable base salts are formed from bases which form non-toxic salts. Illustrative examples include the arginine, benzathine, calcium, choline, diethylamine, diolamine, glycine, lysine, magnesium, meglumine, olamine, potassium, sodium, tromethamine and zinc salts. Hemisalts of acids and bases also can be formed, for example, hemisulphate and hemicalcium salts.

### Targeting Moieties

The identity of the first or second targeting moiety is limited only in that each should be recognized and bound by the CAR, preferably with specificity, have a relatively low molecular weight, and bind to the CAR with high affinity, such as, and without limitation, in a sub-nanomolar range.

In various embodiments, the first targeting moiety in the adaptor compound and the second targeting moiety in the activity modifying compound have the same structure. In other embodiments, the first targeting moiety and the second targeting moiety have different structures.

Examples of the first targeting moiety of the one or more adaptor compounds (or the pharmaceutically acceptable salts thereof) and the second targeting moiety in the activity modifying compound that bind to the CAR expressed by CAR-T cells can include, for example, DNP, TNP, biotin, digoxigenin, fluorescein, FITC, NHS-fluorescein, pentafluorophenyl ester, tetrafluorophenyl ester, a knottin, a centyrin, a DARPin, an affibody, an affilin, an anticalin, an atrimer, an avimer, a bicicyclic peptide, an FN3 scaffold, a cys-knot, a fynomer, a Kunitz domain, or an Obody. In various aspects, exemplary first or second targeting moieties are haptens, including small molecular weight organic molecules.

The first and second targeting moieties hereof can have the same or different structures. In at least one embodiment where the activity modifying compound comprises a rejuvenating compound, the first targeting moiety in the one or more adaptor compound (or pharmaceutically acceptable salt(s) thereof) and the second targeting moiety in the activity modifying compound have the same structure. In another embodiment where the activity modifying compound comprises an immunosuppressive compound, the first targeting moiety in the one or more adaptor compound (or the pharmaceutically acceptable salt(s) thereof) and the second targeting moiety in the activity modifying compound have the same structure.

In at least one illustrative embodiment, the first and/or the second targeting moieties can have the following illustrative structure:

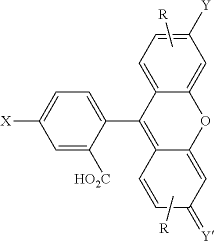

where:
X is oxygen, nitrogen, or sulfur, and where X is attached to linker L;
Y is OR^{a}, NR^{a}₂, or NR^{a}₃⁺; and Y′ is O, NR^{a}, or NR^{a}₂⁺;
each R is independently selected in each instance from H, a fluoro, sulfonic acid, sulfonate, and salts thereof, and the like; and
R^{a} is hydrogen or alkyl.

### Linkers

As noted above, both the adaptor compound and the activity modifying compound can have a first linker and a second linker, respectively. The first and second linkers can have the same or different structures.

The term “linker” includes a chain of atoms that is bio-functionally adapted to form a chemical bond with a small molecule ligand of an adaptor compound or an activity modifying compound (each an “active compound”) and/or a targeting moiety (e.g., the first or second targeting moieties) and connects two or more parts of a molecule to form a compound. Illustratively, the chain of atoms may be selected from carbon (C), nitrogen (N), oxygen (O), sulfur (S), silicon (Si), and phosphorus (P), or C, N, O, S, and P, C, N, O, and S. The chain of atoms may covalently connect different functional capabilities, such as the small molecule ligand and the targeting moiety. The linker (e.g., the first or second linker) may comprise a wide variety of links, such as in the range from about 2 to about 2,000 atoms in the contiguous backbone and can comprise a releasable or non-releasable linker.

The term “releasable” in the context of a linker means a linker that includes at least one bond that can be broken (e.g., chemically or enzymatically hydrolyzed) under physiological conditions, such as, for example, by reducing agent-labile, pH-labile, acid-labile, base-labile, oxidatively labile, metabolically labile, biochemically labile, enzyme-labile or p-aminobenzylic based multivalent releasable bond. It is appreciated that the physiological conditions resulting in bond breaking do not necessarily include a biological or metabolic process and instead may include a standard chemical reaction, such as a hydrolysis reaction for example, at physiological pH or as a result of compartmentalization into a cellular organelle, such as an endosome, having a lower pH than cytosolic pH. A cleavable bond can connect two adjacent atoms within the releasable linker and/or connect other linker portions or the targeting moiety and/or active component, as described herein, for example, at either or both ends of the releasable linker. In some instances, the releasable linker is broken into two or more fragments. In some instances, the releasable linker is separated from the targeting moiety.

In contrast, the term “non-releasable” in the context of a linker means a linker that includes at least one bond that is not easily or quickly broken under physiological conditions. In some embodiments, a non-releasable linker comprises a backbone that is stable under physiological conditions (e.g., the backbone is not susceptible to hydrolysis (e.g., aqueous hydrolysis or enzymatic hydrolysis)). In some embodiments, an activity modifying compound or adaptor compound provided herein comprising a non-releasable linker does not release from the targeting moiety. In some embodiments, the non-releasable linker lacks a disulfide bond (e.g., S—S) or an ester in the backbone. In some embodiments, the compounds comprise a targeting moiety and an active component connected by a backbone that is substantially stable for the entire duration of the compound's circulation. The non-releasable linker can comprise: an amide, ester, ether, amine, and/or thioether (e.g., thio-maleimide). While specific examples are provided herein, it will be understood that any molecule(s) can be used in the non-releasable linker provided that at least one bond that is not easily or quickly broken under physiological conditions is formed.

Both releasable and non-releasable linkers can be engineered to optimize biodistribution, bioavailability, and PK/PD (e.g., of the respective compound) and/or to increase uptake (e.g., of the respective compound) into the targeted cell pursuant to methodologies commonly known in the art or hereinafter developed such as through PEGlaytion and the like.

The first linker in each adaptor compound (or the pharmaceutically acceptable salt thereof) or the second linker in the activity modifying compound (or the pharmaceutically acceptable salt thereof) can comprise a C₁-C₂₀ alkyl, a polyethylene glycol (PEG), a polyproline, an oligo-(4-piperidine) carboxylic acid, an oligo piperidine, a peptide, a saccharo-peptide, a hydrophilic amino acid, a sugar, an unnatural peptidoglycan, a polyvinylpyrrolidone, a pluronic F-127, or a combination thereof. In another embodiment, the linker does not comprise a peptide epitope. Additionally, the linker can further comprise a chemical moiety between the small molecule ligand and the first targeting moiety.

In certain embodiments hereof, the first linker (L) of an adaptor compound (or pharmaceutically acceptable salt thereof) and/or the second linker of the activity modifying compound (or pharmaceutically acceptable salt thereof) comprises a structure having the formula:

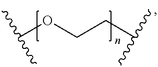

wherein n is an integer from 0 to 200. In another embodiment, n can be an integer from 0 to 150, 0 to 110, 0 to 100, 0 to 90, 0 to 80, 0 to 70, 0 to 60, 0 to 50, 0 to 40, 0 to 30, 0 to 20, 0 to 15, 0 to 14, 0 to 13, 0 to 12, 0 to 11, 0 to 10, 0 to 9, 0 to 8, 0 to 7, 0 to 6, 0 to 5, 0 to 4, 0 to 3, 0 to 2, 0 to 1, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 15 to 31, 15 to 32, 15 to 33, 15 to 34, 15 to 35, 15 to 36, 15 to 37, 15 to 38, 15 to 39, 15 to 40, 15 to 50, 15 to 60, 15 to 70, 15 to 80, 15 to 90, 15 to 100, 15 to 110, 15 to 120, 15 to 130, 15 to 140, 15 to 150, or n can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 80, 90, 100, 108, 110, 120, 130, 140, or 150.

The first linker in the adaptor compound, or the pharmaceutically acceptable salt thereof, and/or the second linker in activity modifying compound, or the pharmaceutically acceptable salt thereof, can be a direct linkage (e.g., a reaction between the isothiocyanate group of FITC and a free amine group of a small molecule ligand for the adaptor compound) or the linkage can be through an intermediary linker. In one embodiment, if present, an intermediary linker can be any biocompatible linker known in the art, such as a divalent linker. In one illustrative embodiment, the divalent linker can comprise about 1 to about 30 carbon atoms. In another illustrative embodiment, the divalent linker can comprise about 2 to about 20 carbon atoms. In other embodiments, lower molecular weight divalent linkers (i.e., those having an approximate molecular weight of about 30 Daltons to about 300 Daltons) are employed. In another embodiment, linker lengths that are suitable include, but are not limited to, linkers having 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, or more atoms.

In another embodiment, the first or the second linker can be a divalent linker that may include one or more linkers. Illustrative linkers are shown in the following table, where * indicates the point of attachment to the small molecule ligand of the adaptor compound, to the first targeting moiety of the adaptor compound, to the second targeting moiety of the activity modifying compound, to the rejuvenating compound, to the immunosuppressive compound, or to other divalent first or second linker portions.

TABLE 1
Linker Formulae

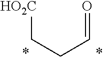

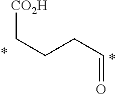

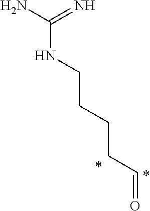

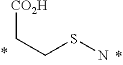

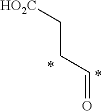

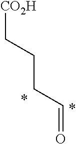

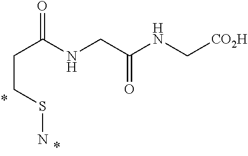 [SMILES: NSCCC(=O)NCC(=O)NCC(=O)O]

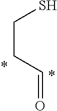

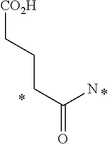

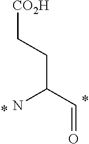

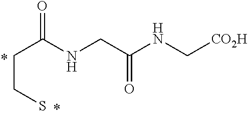 [SMILES: O=C(O)CNC(=O)CNC(=O)CCS]

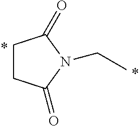

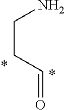

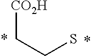

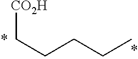

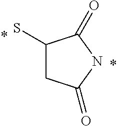

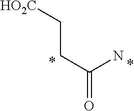

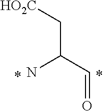

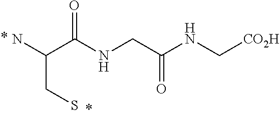 [SMILES: NC(CS)C(=O)NCC(=O)NCC(=O)O]

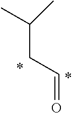

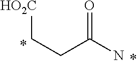

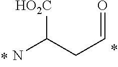

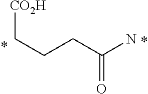

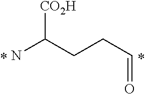

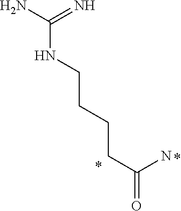 [SMILES: N=C(N)NCCCCC(N)=O]

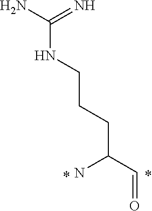 [SMILES: N=C(N)NCCCC(N)C=O]

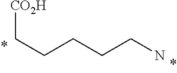

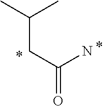

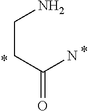

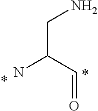

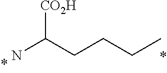

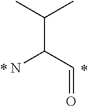

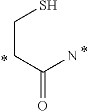

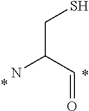

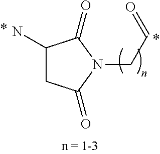

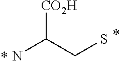

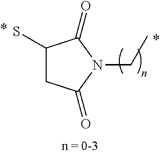

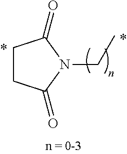

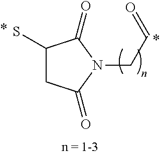

In another embodiment, the first linker in the adaptor compound, or the pharmaceutically acceptable salt thereof, or the second linker in the activity modifying compound (a rejuvenating compound or an immunosuppressive compound), or the pharmaceutically acceptable salt thereof, can each comprise a linker moiety that has a structure selected from the following formulae:

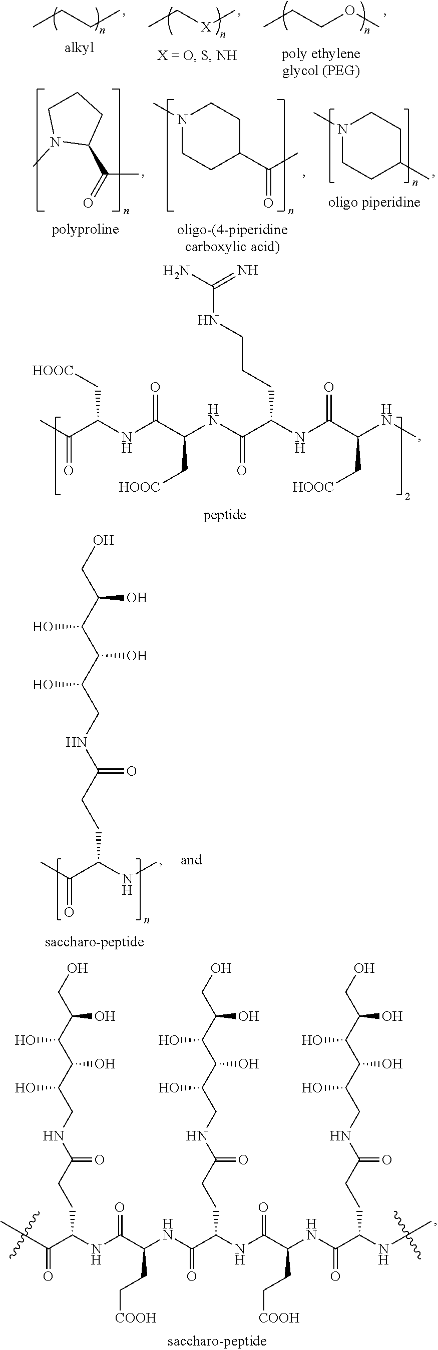

wherein n is an integer from 0 to 200.

The compounds (e.g., the adaptor compounds, the activity modifying compounds, and pharmaceutical salts thereof) can be prepared by conventional methods of organic synthesis practiced by those skilled in the art. Descriptions of compounds are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art, thereby avoiding inherently unstable compounds.

In certain embodiments, the adaptor compound(s), the activity modifying compound, and/or the pharmaceutically acceptable salt of either of the foregoing can contain one or more chiral centers, or can exist as multiple stereoisomers. Accordingly, various embodiments include pure stereoisomers as well as mixtures of stereoisomers, such as enantiomers, diastereomers, and enantiomerically or diastereomerically enriched mixtures.

Additionally, in at least one embodiment, the adaptor compound(s), the activity modifying compound, and/or the pharmaceutically acceptable salt of either of the foregoing can exist as geometric isomers. Accordingly, various embodiments can include pure geometric isomers or mixtures of geometric isomers of the compounds.

The adaptor compound(s), the activity modifying compound, and/or the pharmaceutically acceptable salt of either of the foregoing can also exist in unsolvated forms as well as solvated forms, including hydrated forms. In general, the solvated forms are equivalent to unsolvated forms and are encompassed within the scope of the present disclosure.

### CAR-T Cell Compositions

The compositions and methods can include engineered CAR-T cell compositions. In at least one embodiment, T lymphocytes (e.g., cytotoxic T lymphocytes) are engineered to express CAR that recognizes and binds to the first targeting moiety (e.g., FITC, DNP, or TNP) of the bridge (i.e., the adaptor compound or the pharmaceutically acceptable salt thereof) or the second targeting moiety of the activity modifying compound.

In at least one embodiment, the CARs comprise three domains including 1) a recognition region (e.g., a scFv region of an antibody, a Fab fragment, or the like) that recognizes and binds to the first or second targeting moiety with specificity, 2) a co-stimulation domain that enhances the proliferation and survival of the T lymphocytes, and 3) an activation signaling domain that generates a T lymphocyte activation signal.

Where the recognition region of the CAR comprises a scFv region, the scFv region can be prepared from (i) an antibody known in the art that binds a first or a second targeting moiety, (ii) an antibody newly prepared using a selected first or second targeting moiety, such as a hapten, and (iii) sequence variants derived from the scFv regions of such antibodies, e.g., scFv regions having at least about 80%, at least about 90%, at least about 91%, at least about 92%, at least about 93%, at least about 94%, at least about 95%, at least about 96%, at least about 97%, at least about 98%, at least about 99%, or at least about 99.5% sequence identity with the amino acid sequence of the scFv region from which they are derived.

“Percent (%) sequence identity” with respect to a reference to a polypeptide sequence is defined as the percentage of amino acid or nucleic acid residues, respectively, in a candidate sequence that are identical with the residues in the reference sequence, after aligning the sequences and introducing gaps, if necessary, to achieve the maximum percent sequence identity and not considering any conservative substitutions as part of the sequence identity. Alignment for purposes of determining percent sequence identity can be achieved in various ways that are within the skill of the art, for instance, using publicly available computer software. For example, determination of percent identity or similarity between sequences can be done, for example, by using the GAP program (Genetics Computer Group, software; now available via Accelrys on http://www.accelrys.com), and alignments can be done using, for example, the ClustalW algorithm (VNTI software, InforMax Inc.). Further, a sequence database can be searched using the nucleic acid or amino acid sequence of interest. Algorithms for database searching are typically based on the BLAST software (Altschul et al., 1990), but those skilled in the art can determine appropriate parameters for aligning sequences, including any algorithms needed to achieve maximal alignment over the full length of the sequences being compared. In some embodiments, the percent identity can be determined along the full-length of the nucleic acid or amino acid sequence.

In certain embodiments, the co-stimulation domain of a CAR can serve to enhance the proliferation and survival of the cytotoxic T lymphocytes upon binding of the CAR to a first or a second targeting moiety. Suitable co-stimulation domains include, but are not limited to, CD28, CD137 (4-1BB), a member of the tumor necrosis factor (TNF) receptor family, CD134 (OX40), a member of the TNFR-superfamily of receptors, CD27, CD30, CD150, a DNAX-activating protein of 10 KDa (DAP10), NKG2D, and CD278 (ICOS), a CD28-superfamily co-stimulatory molecule expressed on activated T cells, or combinations thereof. A skilled artisan will understand that sequence variants of these co-stimulation domains can be used without adversely impacting the invention, where the variants have the same or similar activity as the domain upon which they are modeled. In various embodiments, such variants can have at least about 80%, at least about 90%, at least about 91%, at least about 92%, at least about 93%, at least about 94%, at least about 95%, at least about 96%, at least about 97%, at least about 98%, at least about 99%, or at least about 99.5% sequence identity to the amino acid sequence of the domain from which they are derived.

In an illustrative embodiment, the activation signaling domain serves to activate T lymphocytes (e.g., cytotoxic T lymphocytes) upon binding of the CAR to a first or second targeting moiety. Suitable activation signaling domains include, without limitation, the T cell CD3ζ chain, CD3 delta receptor protein, mbl receptor protein, B29 receptor protein, and Fc receptor γ. The skilled artisan will understand that sequence variants of these activation signaling domains can be used where the variants have the same or similar activity as the domain upon which they are modeled. In various embodiments, the variants have at least about 80%, at least about 90%, at least about 91%, at least about 92%, at least about 93%, at least about 94%, at least about 95%, at least about 96%, at least about 97%, at least about 98%, at least about 99%, or at least about 99.5% sequence identity with the amino acid sequence of the domain from which they are derived.

Constructs encoding the CARs can be prepared using genetic engineering techniques. Such techniques are described in detail in Sambrook et al., “Molecular Cloning: A Laboratory Manual,” 3rd Edition, Cold Spring Harbor Laboratory Press, (2001), and Green and Sambrook, “Molecular Cloning: A Laboratory Manual,” 4th Edition, Cold Spring Harbor Laboratory Press, (2012), which are both incorporated herein by reference in their entireties (collectively, the “Protocols”).

By way of non-limiting examples, a plasmid or viral expression vector (e.g., a lentiviral vector, a retrovirus vector, sleeping beauty, and piggyback (transposon/transposase systems that include a non-viral mediated CAR gene delivery system)) can be prepared that encodes a fusion protein comprising a recognition region, one or more co-stimulation domains, and an activation signaling domain, in frame and linked in a 5′ to 3′ direction.

Other arrangements are also acceptable and include a recognition region, an activation signaling domain, and one or more co-stimulation domains.

The term “vector” means any nucleic acid that functions to carry, harbor, or express a nucleic acid of interest. Nucleic acid vectors can have specialized functions such as expression, packaging, pseudotyping, or transduction. Vectors can also have manipulatory functions if adapted for use as a cloning or shuttle vector. The structure of the vector can include any desired form that is feasible to make and desirable for a particular use. Such for can include, for example, circular forms such as plasmids and phagemids, as well as linear or branched forms. A nucleic acid vector can be composed of, or example, DNA or RNA, as well as contain partially or fully, nucleotide derivatives, analogs or mimetics. Such vectors can be obtained from natural sources, produced recombinantly or chemically synthesized.

The placement of the recognition region in the fusion protein will generally be such that display of the region on the exterior of the cell is achieved. Where desired, the CARs can also include additional elements, such as a signal peptide (e.g., CD8α signal peptide) to ensure proper export of the fusion protein to the cell surface, a transmembrane domain to ensure the fusion protein is maintained as an integral membrane protein (e.g., CD8α transmembrane domain, CD28 transmembrane domain, or CD3ζ transmembrane domain), and a hinge domain (e.g., CD8α hinge) that imparts flexibility to the recognition region and allows strong binding to the targeting moiety.

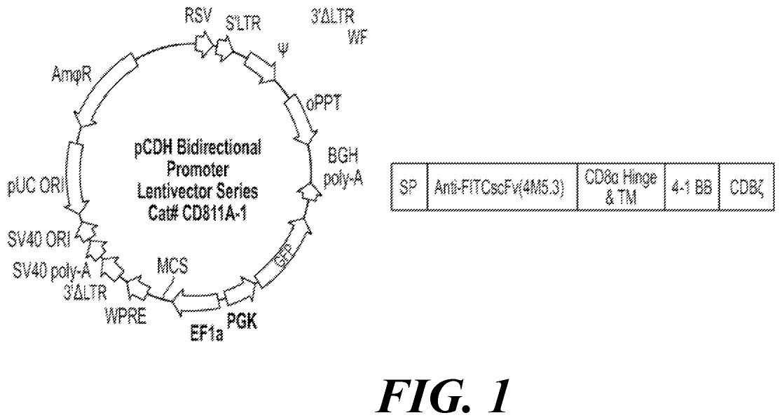

A diagram of an exemplary CAR is shown in FIG. 1 where the fusion protein sequence is incorporated into a lentivirus expression vector and where “SP” is a signal peptide, the CAR is an anti-FITC CAR, a CD8α hinge and a CD8α transmembrane domain are present, the co-stimulation domain is 4-1BB, and the activation signaling domain is CD3ζ. Exemplary nucleic acid sequences of a CAR insert are provided as SEQ ID NOS: 1 and 3, and the encoded amino acid sequence is provided as SEQ ID NO: 2. In yet another embodiment, SEQ ID NO: 2 can comprise or consist of humanized, or human amino acid sequences.

In at least one embodiment, the CAR has a recognition region comprising a scFv region of an anti-FITC antibody, a co-stimulation domain and the co-stimulation domain is CD137 (4-1BB), and an activation signaling domain and the activation signaling domain is a T cell CD3ζ chain. It is well-known to the skilled artisan that an anti-FITC scFv and an anti-fluorescein scFv are equivalent terms.

T lymphocytes (e.g., cytotoxic T lymphocytes) can be genetically engineered to express CAR constructs by transfecting a population of the T lymphocytes with an expression vector encoding the CAR construct. Suitable methods for preparing a transduced population of T lymphocytes expressing a selected CAR construct are well-known to the skilled artisan and are described in at least the Protocols.

In at least one embodiment, CAR-T cells comprising a nucleic acid of SEQ ID NO: 1 or 3 are used. In another embodiment, CAR-T cells comprising a polypeptide of SEQ ID NO: 2 are used. In another aspect, a lentiviral vector is used comprising SEQ ID NO: 1 or 3. In yet another embodiment, SEQ ID NO: 2 can comprise or consist of humanized or human amino acid sequences.

In each of these embodiments, variant nucleic acid sequences or amino acid sequences having at least about 80%, at least about 90%, at least about 95%, at least about 97%, at least about 98%, at least about 99%, or at least about 99.5% sequence identity to SEQ ID NOS: 1 to 3 can be used. In another embodiment, the nucleic acid sequence can be a variant nucleic acid sequence having at least about 80%, at least about 90%, at least about 95%, at least about 97%, at least about 98%, at least about 99%, or at least about 99.5% sequence identity to SEQ ID NOS: 1 or 2 as long as the variant sequence encodes a polypeptide of SEQ ID NO: 2. In another embodiment, the nucleic acid sequence or the amino acid sequence can be a variant nucleic acid or amino acid sequence having at least about 80%, at least about 90%, at least about 95%, at least about 97%, at least about 98%, at least about 99%, or at least about 99.5% sequence identity to SEQ ID NO: 1 or 3 along a stretch of 200 nucleic acids or, for SEQ ID NO: 2, along a stretch of 200 amino acids.

In one embodiment, the T lymphocytes (e.g., cytotoxic T lymphocytes used to prepare CAR-T cells) used in the methods described herein, can be autologous cells, although heterologous cells can also be used, such as when the patient being treated has received high-dose chemotherapy or radiation treatment to destroy the patient's immune system. In one embodiment, allogenic cells can be used.

The T lymphocytes can be obtained from a subject by means well-known in the art. For example, T cells (e.g., cytotoxic T cells) can be obtained by collecting peripheral blood from the subject, subjecting the blood to Ficoll density gradient centrifugation, and then using a negative T cell isolation kit (such as EasySep™ T Cell Isolation Kit) to isolate a population of T cells from the peripheral blood.

In certain embodiments, the population of T lymphocytes (e.g., cytotoxic T cells) need not be pure and may contain other cells, such as other types of T cells (in the case of cytotoxic T cells, for example), monocytes, macrophages, natural killer cells, and B cells. Further, in at least one embodiment, the population being collected can comprise at least about 90% of the selected cell type, at least about 91%, 92%, 93%, 94%, 95%, 96%, 97%, 98%, 99%, or 100% of the selected cell type.

Generally, after the T lymphocytes are obtained, the cells are cultured under conditions that promote the activation of the cells. In at least one embodiment, the culture conditions are such that the cells can be administered to a subject without concern for reactivity against components of the culture medium. For example, the culture conditions may not include bovine serum products, such as bovine serum albumin. In one aspect, the activation can be achieved by introducing known activators into the culture medium, such as anti-CD3 antibodies in the case of cytotoxic T cells. Other suitable activators are generally known and include, for example, anti-CD28 antibodies. The population of lymphocytes can be cultured under conditions promoting activation for about 1 to about 4 days, for example. The appropriate level of activation can be determined by cell size, proliferation rate, or activation markers determined by flow cytometry.

In at least one embodiment, after the population of T lymphocytes has been cultured under conditions promoting activation, the cells are transfected with an expression vector encoding a CAR. Suitable vectors and transfection methods for use in various embodiments are described above. After transfection, the cells can be immediately administered to the patient or the cells can be cultured for a time period to allow time for the cells to recover from the transfection, for example, at least about 1, 2, 3, 4, 5, 6, 7, 8, 9, 10, 11, 12, 13, 14, 15, 16, 17, 18 or more days, or between about 5 and about 12 days, between about 6 and about 13 days, between about 7 and about 14 days, or between about 8 and about 15 days. In one aspect, suitable culture conditions can be similar to the conditions under which the cells were cultured for activation either with or without the agent that was used to promote activation.

Thus, as described above, the methods of treatment described herein can further comprise 1) obtaining a population of autologous or heterologous T lymphocytes (e.g., cytotoxic T lymphocytes used to prepare CAR-T cells), 2) culturing the T lymphocytes under conditions that promote the activation of the cells, and 3) transfecting the lymphocytes with an expression vector encoding a CAR to form CAR-T cells.

When the cells have been transfected and activated, a composition comprising the CAR-T cells can be prepared and administered to the subject. In at least one embodiment, culture media that lacks any animal products, such as bovine serum, can be used to culture the CAR-T cells. In another embodiment, tissue culture conditions typically used by the skilled artisan to avoid contamination with bacteria, fungi and mycoplasma can be used. In certain embodiments, prior to being administered to a patient, the cells (e.g., CAR-T cells) are pelleted, washed, and are resuspended in a pharmaceutically acceptable carrier or diluent.

Exemplary compositions comprising CAR-expressing T lymphocytes (e.g., cytotoxic T lymphocytes) include compositions comprising the cells in sterile 290 mOsm saline, in infusible cryomedia (containing Plasma-Lyte A, dextrose, sodium chloride injection, human serum albumin and DMSO), in 0.9% NaCl with 2% human serum albumin, or in any other sterile 290 mOsm infusible materials. Alternatively, in another embodiment, depending on the identity of the culture medium, the CAR-T cells can be administered in the culture media as the composition, or concentrated and resuspended in the culture medium before administration. In various embodiments, the CAR-T cell composition can be administered to the subject via any suitable means, such as parenteral administration, e.g., intradermally, subcutaneously, intramuscularly, intraperitoneally, intravenously, or intrathecally.

In one aspect, the total number of CAR-T cells and the concentration of the cells in the composition administered to the patient will vary depending on a number of factors including the type of T lymphocytes (e.g., cytotoxic T lymphocytes) being used, the binding specificity of the CAR, the identity of the first or second or third targeting moiety and the small molecule ligand, the rejuvenating compound, or the immunosuppressive compound, the identity of the cancer, the location of the cancer in the patient, the means used to administer the compositions to the patient, and the health, age and weight of the patient being treated. In various embodiments, suitable compositions comprising transduced CAR-T cells include those having a volume of about 0.1 ml to about 200 ml and about 0.1 ml to about 125 ml.

### Vector Compositions

Certain embodiments of the compositions and methods can include vector compositions. In some embodiments, the composition comprises a vector comprising a promoter operatively linked to a nucleic acid sequence encoding a CAR construct described herein (for example, and without limitation, SEQ ID NO: 1 or 3). In some embodiments, the vector composition comprises lentiviral particles that carry a nucleic acid sequence encoding a CAR described herein. In some embodiments, the vector composition comprises a therapeutically effective amount of such lentiviral particles.

A lentivirus is a non-limiting example of a vector system that can be used. Lentiviruses are complex retroviruses that, in addition to the common retroviral genes Gag, Pol and Env, contain other genes with regulatory or structural function. The higher complexity enables the virus to modulate its life cycle, as in the course of latent infection. Some examples of lentivirus include the Human Immunodeficiency Viruses (HIV-1 and HIV-2) and the Simian Immunodeficiency Virus (SIV). Lentiviral vectors have been generated by multiply attenuating the HIV virulence genes, for example, the genes Env, Vif, Vpr, Vpu and Nef are deleted, making the vector biologically safe.

Lentiviral vectors offer many advantages for gene therapy. Unless engineered to be non-integrating, lentiviral vectors integrate stably into chromosomes of target cells, permitting long-term expression of delivered transgenes. Further, they do not transfer viral genes thus avoiding the problem of generating transduced cells that can be destroyed by cytotoxic T cells. Further, they have a relatively large cloning capacity, sufficient for most envisioned clinical applications. Among retroviruses, lentiviruses have the unique ability to integrate their genome into the chromatin of nondividing cells. This is especially important in the context of gene-therapy for tissues, for example, in the hematopoietic system, the brain, liver, lungs, and muscle. For example, vectors derived from HIV-1 allow efficient in vivo and ex vivo delivery, integration and stable expression of transgenes into cells such as neurons, hepatocytes, and myocytes (Blomer et al., 1997; Kafri et al., 1997; Naldini et al., 1996; Naldini et al., 1998).

Lentiviral vectors are known in the art. For example, see Naldini et al. (1996) Science 272: 263-267; Zufferey et al. (1998) J. Virol. 72: 9873-9880; Dull et al. (1998) J. Virol. 72: 8463-8471; U.S. Pat. Nos. 6,013,516; 5,994,136. Generally, these vectors are configured to carry the essential sequences for selection of cells containing the vector, for incorporating foreign nucleic acid into a lentiviral particle, and for transfer of the nucleic acid into a target cell.

A commonly used lentiviral vector system is the so-called third-generation system. Third-generation lentiviral vector systems can include four plasmids. The “transfer plasmid” encodes the polynucleotide sequence that is delivered by the lentiviral vector system to the target cell. The transfer plasmid generally has one or more transgene sequences of interest flanked by long terminal repeat (LTR) sequences that facilitate integration of the transfer plasmid sequences into the host genome. For safety reasons, transfer plasmids are generally designed to make the resulting vector replication incompetent. For example, the transfer plasmid lacks gene elements necessary for generation of infective particles in the host cell. Additionally, the transfer plasmid can be designed with a deletion of the 3′ LTF, rendering the virus “self-inactivating” (SIN). See Dull et al. (1998) J. Virol. 72:8463-8471; Miyoshi et al. (1998) J. Virol. 72:8150-8157.

Third-generation systems also generally include two “packaging plasmids” and an “envelope plasmid.” The “envelope plasmid” generally encodes an Env gene operatively linked to a promoter. In at least one embodiment of a third-generation system, the Env gene is VSV-G and the promoter is the CMV promoter. The third-generation system uses two packaging plasmids, one encoding Gag and Pol and the other encoding Rev as a further safety feature—an improvement over the single packaging plasmid of so-called second-generation systems. Although safer, the third-generation system can be more cumbersome to use and result in lower viral titers due to the addition of an additional plasmid. Examplary packing plasmids including, without limitation, pMD2.G, pRSV-rev, pMDLG-pRRE, and pRRL-GOI.

In some instances, lentiviral vector systems rely on the use of a “packaging cell line.” In general, the packaging cell line is a cell line whose cells are capable of producing infectious lentiviral particles when the transfer plasmid, packaging plasmid(s), and envelope plasmid are introduced into the cells. Various methods of introducing the plasmids into the cells can be used, including transfection or electroporation. In some cases, a packaging cell line is adapted for high-efficiency packaging of a lentiviral vector system into lentiviral particles.

As used herein, the term “lentiviral vector” means a nucleic acid that encodes a lentiviral cis nucleic acid sequence required for genome packaging. A lentiviral vector also can encode other cis nucleic acid sequences beneficial for gene delivery, including for example, cis sequences required for reverse transcription, proviral integration or genome transcription. A lentiviral vector performs transduction functions of a lentiviral vector. As such, the exact makeup of a vector genome will depend on the genetic material desired to be introduced into a target cell. Therefore, a vector genome can encode, for example, additional polypeptides or functions other than that required for packaging, reverse transcription, integration, or transcription. Such functions generally include coding for cis elements required for expression of a nucleic acid of interest. The lentiviral cis sequences or elements can be derived from a lentivirus genome or other virus or vector genome so long as the lentiviral vector genome can be packaged by a packaging cell line into a lentiviral particle and introduced into a target cell. In some embodiments, the target cell for the lentiviral vector is an immune cell. In some embodiments, the target immune cell is a T cell or NK cell. In some embodiments, the target immune cell exists in a tumor microenvironment.

The lentiviral particles produced generally include an RNA genome (e.g., derived from a transfer plasmid), a lipid-bilayer envelope in which the Env protein is embedded, and other accessory proteins including integrase, protease, and matrix protein. As used herein, the term “lentiviral particle” means a viral particle that includes an envelope, has one or more characteristics of a lentivirus, and is capable of invading a target host cell (e.g., a T cell or NK cell). Such characteristics can include, for example, infecting non-dividing host cells, transducing non-dividing host cells, infecting or transducing host immune cells, containing a lentiviral virion including one or more of the gag structural polypeptides p7, p24, and p17, containing a lentiviral envelope including one or more of the env encoded glycoproteins p41, p120, and p160, containing a genome including one or more lentivirus cis-acting sequences functioning in replication, proviral integration or transcription, containing a genome encoding a lentiviral protease, reverse transcriptase or integrase, or containing a genome encoding regulatory activities such as Tat or Rev. Detailed description of lentiviral vectors and lentiviral particles is provided in International Publication No. WO 2019/200056, which is incorporated by reference herein in its entirety.

Lentiviral vectors can be used to encode T cell activation receptors. A “T cell activation receptor” means one or more transmembrane proteins that are configured to be expressed on the cell surface of transduced cells such that the T cell activation receptor provides a mitogenic signal to the transduced cell. A T cell activation receptor is used because the target cells, in most cases, are T cells. The present methods can be adapted for use with other cell types by use of an activation receptor that retains activity in another cell type. T cell activation receptors useful here can include a signaling domain that is a cytokine receptor signaling domain, a co-stimulatory receptor signaling domain, a T cell receptor subunit signaling domain, a growth factor receptor signaling domain, or the like (e.g., as previously described in connection with the CAR compositions).

It can be advantageous, in some cases, to provide a means to target transduced cells to particular cells or tissues. Accordingly, the lentiviral vector can comprise (instead of or in addition to other genes) a polynucleotide encoding a CAR described herein (e.g., those directed to the first and/or second targeting moieties and, for example, inducibly dimerize the small molecule ligand or compound linked therewith).

As is known, lentiviral vectors can further comprise promoters and/or enhancers specific to T cells. In some cases, promoters can be used to control expression of the T cell activation receptor. Further, lentiviral vectors can include fusion glycoproteins (e.g., for pseudotyping purposes). See, e.g., Joglekar et al. (2017) Human Gene Therapy Methods 28:291-301. In certain embodiments, pseudotyping a fusion glycoprotein or functional variant thereof facilitates targeting transduction of specific cell types including, without limitation, T cells.

In certain embodiments, vectors hereof can include the Woodchuck Hepatitis Virus Posttranscriptional Regulatory Element (wPRE) or a nucleic acid sequence substantially identical to wPRE. See U.S. Pat. No. 6,136,597; Lee et al. (2005) Exp Physiol. 90:33-37. Variants of the wPRE element with reduced size are known in the art. wPRE-O refers to a variant of wPRE with the intermediate size. In some embodiments, the wPRE sequence increases expression of genes delivered by such viral vectors.

In some cases, lentiviral vectors can comprise a polynucleotide sequence encoding the 2A peptide. The term “2A peptide” refers to a self-cleaving peptide configured to generate two or more proteins from a single open reading frame. 2A peptides are 18-22 residue-long viral oligopeptides that mediate “cleavage” of polypeptides during translation in eukaryotic cells. “2A peptide” can refer to peptides with various amino acid sequences. Detailed methodology for design and use of 2A peptides is provided by Szymczak-Workman et al. (2012) Cold Spring Harb. Protoc. 2012: 199-204.

In some embodiments, vector compositions are administered directly to the subject. In some embodiments, vector compositions are administered in conjunction with T cells. In some embodiments, vector compositions and T cells are separately administered. In some embodiments, T cells are activated and transduced in vivo by administered vector compositions.

### Combinations, Compositions and Methods for Treating Cancer

Combinations and compositions for treating a cancer are also provided. As used herein, the term “combination” generally refers to any product comprising more than one ingredient, including one or more of the compounds described herein (e.g., an adaptor compound, an activity modifying compound (e.g., a rejuvenating compound or an immunosuppressive compound), or a pharmaceutically acceptable salt of the foregoing). It is to be understood that the compositions described herein can be prepared from isolated compounds or from salts, solutions, hydrates, solvates, and other forms of the compounds. It is appreciated that certain functional groups, such as the hydroxy, amino, and like groups, can form complexes with water and/or various solvents, in the various physical forms of the compounds. It will also be understood that, in certain circumstances, the compounds (and compositions comprising the compounds) can be prepared from various amorphous, non-amorphous, partially crystalline, crystalline, and/or other morphological forms of the compounds, and the compositions can be prepared from various hydrates and/or solvates of the compounds. Accordingly, pharmaceutical compositions that recite the compounds described herein include each of, or any combination of, or individual forms of, the various morphological forms and/or solvate or hydrate forms of the compounds.

A combination for modifying T cell activity in a subject having cancer and/or treating cancer comprises one or more adaptor compounds, or pharmaceutically acceptable salts thereof, and an activity modifying compound (e.g., a rejuvenating compound, an immunosuppressive compound, or a pharmaceutically acceptable salt of either of the foregoing) is also provided. Such compounds can be any of the like compounds and, in at least one embodiment, each adaptor compound comprises a small molecule ligand linked to a first targeting moiety by a first linker, and the activity modifying compound comprises a rejuvenating compound or an immunosuppressive compound linked to a second targeting moiety via a second linker. Additionally, in at least one embodiment, the combination can further comprise a composition comprising CAR-T cells expressing a CAR or a vector comprising a promoter operatively linked to a nucleic acid sequence encoding the CAR (for example, SEQ ID NO: 1 or 3), with the CAR directed to the first targeting moiety, the second targeting moiety, or both the first and second targeting moieties.

In yet another embodiment, a combination for treating a cancer (for example, by modifying T cell activity in a subject) is provided. The combination comprises one or more of the compounds and compositions of the present disclosure.

Compounds and compositions can be administered in unit dosage forms and/or compositions containing one or more pharmaceutically acceptable carriers, adjuvants, diluents, excipients, and/or vehicles, and combinations thereof. The term “administering,” and its formatives, generally refer to any and all means of introducing the compounds and compositions described herein (e.g., the CAR-T cell compositions, the adaptor compound(s) or the pharmaceutically acceptable salt(s) thereof, and/or the activity modifying compounds) to a cell, tissue, organ, or biological fluid of a subject.

Administration of the compounds and compositions as salts may be appropriate. Examples of acceptable salts include, without limitation, alkali metal (for example, sodium, potassium or lithium) or alkaline earth metals (for example, calcium) salts; however, any salt that is generally non-toxic and effective when administered to the subject being treated is acceptable.

As used herein, a “subject” is a mammal, preferably a human, but it can also be a non-human animal (including, without limitation, a laboratory, an agricultural, a domestic, or a wild animal). Thus, the methods, compounds, and compositions described herein are applicable to both human and veterinary disease and applications. In various aspects, the subject can be a laboratory animal such as a rodent (e.g., mouse, rat, hamster, etc.), a rabbit, a monkey, a chimpanzee, a domestic animal such as a dog, a cat, or a rabbit, an agricultural animal such as a cow, a horse, a pig, a sheep, or a goat, or a wild animal in captivity such as a bear, a panda, a lion, a tiger, a leopard, an elephant, a zebra, a giraffe, a gorilla, a dolphin, or a whale. In certain embodiments, subjects are “patients,” i.e., living humans or animals that are receiving medical care for a disease or condition, which includes persons or animals with no defined illness who are being evaluated for signs of pathology. In certain embodiments, subjects that can be addressed using the methods hereof include subjects identified or selected as having or being at risk for having cancer. Such identification and/or selection can be made by clinical or diagnostic evaluation.

The compounds and compositions can be formulated as pharmaceutical compositions and/or administered to a subject, such as a human patient, in a variety of forms adapted to the chosen route of administration. Indeed, the adaptor compound, or the pharmaceutically acceptable salt thereof, or the activity modifying compound, or the pharmaceutically acceptable salt thereof, or the CAR-T cell composition, or the vector composition (including, for example, the lentiviral particles hereof) can be administered to a subject using any suitable method known in the art. In one aspect, the adaptor compound, or the pharmaceutically acceptable salt thereof, or the rejuvenating compound, or the pharmaceutically acceptable salt thereof, or the immunosuppressive compound, or the pharmaceutically acceptable salt thereof described herein may be administered in unit dosage forms and/or formulations containing conventional nontoxic pharmaceutically-acceptable carriers, adjuvants, and vehicles.

Further, the adaptor compound(s), or the pharmaceutically acceptable salt(s) thereof, or the rejuvenating compound, or the pharmaceutically acceptable salt thereof, or the immunosuppressive compound, or the pharmaceutically acceptable salt thereof, or the CAR-T cell composition, or the vector composition as described herein can be administered directly into the blood stream, into muscle, or into an internal organ. In various embodiments, suitable routes for such parenteral administration include intravenous, intraarterial, intraperitoneal, intrathecal, epidural, intracerebroventricular, intraurethral, intrasternal, intracranial, intratumoral, intramuscular and subcutaneous delivery. In one embodiment, means for parenteral administration include needle (including microneedle) injectors, needle-free injectors and infusion techniques. It will be appreciated that the compounds and compositions hereof can be formulated for the desired administration modality as well.

For example, parenteral formulations are typically aqueous solutions and can contain carriers or excipients such as salts, carbohydrates and buffering agents (preferably at a pH of from 3 to 9), but can also be formulated, where suitable, as a sterile non-aqueous solution or as a dried form to be used in conjunction with a suitable vehicle such as sterile, pyrogen-free water or sterile saline. In other embodiments, any of the liquid formulations described herein can be adapted for parenteral administration. The preparation under sterile conditions, by lyophilization to produce a sterile lyophilized powder for a parenteral formulation, can readily be accomplished using standard pharmaceutical techniques well-known to those skilled in the art. In one embodiment, the solubility of the adaptor compound, or the pharmaceutically acceptable salt thereof, or the rejuvenating compound, or the pharmaceutically acceptable salt thereof, or the immunosuppressive compound, or the pharmaceutically acceptable salt thereof, or the activity modifying compound, or the pharmaceutically acceptable salt thereof, used in the preparation of a parenteral formulation can be increased by the use of appropriate formulation techniques, such as the incorporation of solubility-enhancing agents.

The pharmaceutical dosage forms of the adaptor compound(s) and/or the activity modifying compound that are suitable for injection or infusion can include sterile aqueous solutions or dispersions or sterile powders comprising the active ingredients that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example and without limitation, water, ethanol, a polyol (e.g., glycerol, propylene glycol, liquid PEG(s), and the like), vegetable oils, nontoxic glyceryl esters, and/or suitable mixtures thereof. In at least one embodiment, the proper fluidity can be maintained by the formation of liposomes, by the maintenance of the required particle size in the case of dispersions or by the use of surfactants. The action of microorganisms can be prevented by the addition of various antibacterial and antifungal agents, such as parabens, chlorobutanol, phenol, sorbic acid, thimerosal, and the like. In certain cases, it can be desirable to include one or more isotonic agents, such as sugars, buffers, or sodium chloride. Prolonged absorption of the injectable compositions can be brought about by the incorporation of agents formulated to delay absorption, for example, aluminum monostearate and gelatin.

Sterile injectable solutions can be prepared by incorporating the active component in the required amount of the appropriate solvent with one or more of the other ingredients set forth above, as required, followed by filter sterilization. In the case of sterile powders for the preparation of sterile injectable solutions, the preferred methods of preparations are vacuum drying and the freeze-drying techniques, which yield a powder of the active ingredient plus any additional desired ingredient present in the previously sterile-filtered solutions.

Useful dosages of the compounds can be determined by comparing their in vitro activity and the in vivo activity in animal models. Methods of the extrapolation of effective dosages in mice and other animals to human subjects are known in the art. Indeed, the dosage of the compound can vary significantly depending on the condition of the subject, the cancer type being treated, how advanced the pathology is, the route of administration of the compound and tissue distribution, and the possibility of co-usage of other therapeutic treatments (such as radiation therapy or additional drugs in combination therapies). The amount of the compositions and/or compound(s) required for use in treatment (e.g., the therapeutically or prophylactically effective amount or dose) will vary not only with the particular application, but also with the salt selected (if applicable) and the characteristics of the subject (such as, for example, age, condition, sex, the subject's body surface area and/or mass, tolerance to drugs) and will ultimately be at the discretion of the attendant physician, clinician, or otherwise. “Therapeutically effective amount” or “prophylactically effective amount” is defined as an amount of a reagent or pharmaceutical composition that is sufficient to induce a desired immune response specific for encoded heterologous antigens or show benefit in a subject (i.e., to cause a decrease, prevention, or treatment of the symptoms of the condition being treated).

In various embodiments, the transduced CAR-T cells administered to the subject can comprise from about 1×10⁵ to about 1×10¹⁵ or 1×10⁶ to about 1×10¹⁵ transduced CAR-T cells. In various embodiments about 1×10⁵ to about 1×10¹⁰, about 1×10⁶ to about 1×10¹⁰, about 1×10⁶ to about 1×10⁹, about 1×10⁶ to about 1×10⁸, about 1×10⁶ to about 2×10⁷, about 1×10⁶ to about 3×10⁷, about 1×10⁶ to about 1.5×10⁷, about 1×10⁶ to about 1×10⁷, about 1×10⁶ to about 9×10⁶, about 1×10⁶ to about 8×10⁶, about 1×10⁶ to about 7×10⁶, about 1×10⁶ to about 6×10⁶, about 1×10⁶ to about 5×10⁶, about 1×10⁶ to about 4×10⁶, about 1×10⁶ to about 3×10⁶, about 1×10⁶ to about 2×10⁶, about 2×10⁶ to about 6×10⁶, about 2×10⁶ to about 5×10⁶, about 3×10⁶ to about 6×10⁶, about 4×10⁶ to about 6×10⁶, about 4×10⁶ to about 1×10⁷, about 1×10⁶ to about 1×10⁷, about 1×10⁶ to about 1.5×10⁷, about 1×10⁶ to about 2×10⁷, about 0.2×10⁶ to about 1×10⁷, about 0.2×10⁶ to about 1.5×10⁷, about 0.2×10⁶ to about 2×10⁷, or about 5×10⁶ CAR-T cells can be administered to the subject. In one aspect, in any embodiment described herein, a single dose or multiple doses of the CAR-T cells can be administered to the subject. In any of the embodiments described in this paragraph, the CAR-T cell dose can be in numbers of CAR-T cells per kg of subject body weight. In any embodiment described herein, the CAR-T cells can be administered before or after the adaptor compound(s), or the pharmaceutically acceptable salt thereof.

In other embodiments, the dose of the CAR-T cells administered to the subject in the CAR-T cell composition is selected from the group consisting of about 1 million, about 2 million, about 3 million, about 4 million, about 5 million, about 6 million, about 7 million, about 8 million, about 9 million, about 10 million, about 11 million, about 12 million, about 12.5 million, about 13 million, about 14 million, and about 15 million of the CAR-T cells. In these embodiments, the CAR-T cell dose can be in numbers of CAR-T cells per kg of subject body weight.

The amount of the one or more adaptor compounds, or the pharmaceutically acceptable salts thereof, or the rejuvenating compound, or the pharmaceutically acceptable salt thereof, or the immunosuppressive compound, or the pharmaceutically acceptable salt thereof, or the activity modifying compound, or the pharmaceutically acceptable salt thereof, to be administered to the subject can vary significantly depending on the cancer being treated, the route of administration of the one or more adaptor compounds, or the pharmaceutically acceptable salts thereof, or the rejuvenating compound, or the pharmaceutically acceptable salt thereof, and the tissue distribution. The amount to be administered to a subject can be based on body surface area, mass, and physician assessment.

In various embodiments, amounts to be administered can range, for example, from about 0.05 mg to about 30 mg, 0.05 mg to about 25.0 mg, about 0.05 mg to about 20.0 mg, about 0.05 mg to about 15.0 mg, about 0.05 mg to about 10.0 mg, about 0.05 mg to about 9.0 mg, about 0.05 mg to about 8.0 mg, about 0.05 mg to about 7.0 mg, about 0.05 mg to about 6.0 mg, about 0.05 mg to about 5.0 mg, about 0.05 mg to about 4.0 mg, about 0.05 mg to about 3.0 mg, about 0.05 mg to about 2.0 mg, about 0.05 mg to about 1.0 mg, about 0.05 mg to about 0.5 mg, about 0.05 mg to about 0.4 mg, about 0.05 mg to about 0.3 mg, about 0.05 mg to about 0.2 mg, about 0.05 mg to about 0.1 mg, about .01 mg to about 2 mg, about 0.3 mg to about 10 mg, about 0.1 mg to about 20 mg, or about 0.8 to about 3 mg. One of skill in the art will readily appreciate that the dose may vary within the various ranges provided above based on the factors noted above and may be at the physician's discretion.

In other embodiments, the dose of the one or more adaptor compounds, or the pharmaceutically acceptable salts thereof, or the rejuvenating compound, or the pharmaceutically acceptable salt thereof, or the immunosuppressive compound, or the pharmaceutically acceptable salt thereof, or the activity modifying compound, or the pharmaceutically acceptable salt thereof, can range, for example, from about 50 nmoles/kg to about 3,000 nmoles/kg of subject body weight, about 50 nmoles/kg to about 2,800 nmoles/kg about 50 nmoles/kg to about 2,600 nmoles/kg about 50 nmoles/kg to about 2,400 nmoles/kg about 50 nmoles/kg to about 2,200 nmoles/kg about 50 nmoles/kg to about 2,100 nmoles/kg about 50 nmoles/kg to about 2,000 nmoles/kg, about 50 nmoles/kg to about 1,000 nmoles/kg, about 50 nmoles/kg to about 900 nmoles/kg, about 50 nmoles/kg to about 800 nmoles/kg, about 50 nmoles/kg to about 700 nmoles/kg, about 50 nmoles/kg to about 600 nmoles/kg, about 50 nmoles/kg to about 500 nmoles/kg, about 50 nmoles/kg to about 400 nmoles/kg, about 50 nmoles/kg to about 300 nmoles/kg, about 50 nmoles/kg to about 200 nmoles/kg, about 50 nmoles/kg to about 100 nmoles/kg, about 100 nmoles/kg to about 300 nmoles/kg, about 100 nmoles/kg to about 500 nmoles/kg, about 100 nmoles/kg to about 1,000 nmoles/kg, about 100 nmoles/kg to about 2,000 nmoles/kg of subject body weight. In other embodiments, the dose may be about 1 nmoles/kg, about 5 nmoles/kg, about 10 nmoles/kg, about 20 nmoles kg, about 25 nmoles/kg, about 30 nmoles/kg, about 40 nmoles/kg, about 50 nmoles/kg, about 60 nmoles/kg, about 70 nmoles/kg, about 80 nmoles/kg, about 90 nmoles/kg, about 100 nmoles/kg, about 150 nmoles/kg, about 200 nmoles/kg, about 250 nmoles/kg, about 300 nmoles/kg, about 350 nmoles/kg, about 400 nmoles/kg, about 450 nmoles/kg, about 500 nmoles/kg, about 600 nmoles/kg, about 700 nmoles/kg, about 800 nmoles/kg, about 900 nmoles/kg, about 1000 nmoles/kg, about 2,000 nmoles/kg, about 2,500 nmoles/kg or about 3,000 nmoles/kg of body weight of the subject. In yet other embodiments, the dose may be about 0.1 nmoles/kg, about 0.2 nmoles/kg, about 0.3 nmoles/kg, about 0.4 nmoles kg, or about 0.5 nmoles/kg, about 0.1 nmoles/kg to about 1000 nmoles/kg, about 0.1 nmoles/kg to about 900 nmoles/kg, about 0.1 nmoles/kg to about 850 nmoles/kg, about 0.1 nmoles/kg to about 800 nmoles/kg, about 0.1 nmoles/kg to about 700 nmoles/kg, about 0.1 nmoles/kg to about 600 nmoles/kg, about 0.1 nmoles/kg to about 500 nmoles/kg, about 0.1 nmoles/kg to about 400 nmoles/kg, about 0.1 nmoles/kg to about 300 nmoles/kg, about 0.1 nmoles/kg to about 200 nmoles/kg, about 0.1 nmoles/kg to about 100 nmoles/kg, about 0.1 nmoles/kg to about 50 nmoles/kg, about 0.1 nmoles/kg to about 10 nmoles/kg, or about 0.1 nmoles/kg to about 1 nmoles/kg of body weight of the subject. In other embodiments, the dose may be about 0.3 nmoles/kg to about 1000 nmoles/kg, about 0.3 nmoles/kg to about 900 nmoles/kg, about 0.3 nmoles/kg to about 850 nmoles/kg, about 0.3 nmoles/kg to about 800 nmoles/kg, about 0.3 nmoles/kg to about 700 nmoles/kg, about 0.3 nmoles/kg to about 600 nmoles/kg, about 0.3 nmoles/kg to about 500 nmoles/kg, about 0.3 nmoles/kg to about 400 nmoles/kg, about 0.3 nmoles/kg to about 300 nmoles/kg, about 0.3 nmoles/kg to about 200 nmoles/kg, about 0.3 nmoles/kg to about 100 nmoles/kg, about 0.3 nmoles/kg to about 50 nmoles/kg, about 0.3 nmoles/kg to about 10 nmoles/kg, or about 0.3 nmoles/kg to about 1 nmoles/kg of body weight of the subject.

In various other embodiments, the dose of the one or more adaptor compounds, or the pharmaceutically acceptable salts thereof, or the rejuvenating compound, or the pharmaceutically acceptable salt thereof, or the immunosuppressive compound, or the pharmaceutically acceptable salt thereof, or the activity modifying compound, or the pharmaceutically acceptable salt thereof, may range from, for example, about 10 nmoles/kg to about 10,000 nmoles/kg, from about 10 nmoles/kg to about 5,000 nmoles/kg, from about 10 nmoles/kg to about 3,000 nmoles/kg, about 10 nmoles/kg to about 2,500 nmoles/kg, about 10 nmoles/kg to about 2,000 nmoles/kg, about 10 nmoles/kg to about 1,000 nmoles/kg, about 10 nmoles/kg to about 900 nmoles/kg, about 10 nmoles/kg to about 800 nmoles/kg, about 10 nmoles/kg to about 700 nmoles/kg, about 10 nmoles/kg to about 600 nmoles/kg, about 10 nmoles/kg to about 500 nmoles/kg, about 10 nmoles/kg to about 400 nmoles/kg, about 10 nmoles/kg to about 300 nmoles/kg, about 10 nmoles/kg to about 200 nmoles/kg, about 10 nmoles/kg to about 150 nmoles/kg, about 10 nmoles/kg to about 100 nmoles/kg, about 10 nmoles/kg to about 90 nmoles/kg, about 10 nmoles/kg to about 80 nmoles/kg, about 10 nmoles/kg to about 70 nmoles/kg, about 10 nmoles/kg to about 60 nmoles/kg, about 10 nmoles/kg to about 50 nmoles/kg, about 10 nmoles/kg to about 40 nmoles/kg, about 10 nmoles/kg to about 30 nmoles/kg, about 10 nmoles/kg to about 20 nmoles/kg, about 200 nmoles/kg to about 900 nmoles/kg, about 200 nmoles/kg to about 800 nmoles/kg, about 200 nmoles/kg to about 700 nmoles/kg, about 200 nmoles/kg to about 600 nmoles/kg, about 200 nmoles/kg to about 500 nmoles/kg, about 250 nmoles/kg to about 600 nmoles/kg, about 300 nmoles/kg to about 600 nmoles/kg, about 300 nmoles/kg to about 500 nmoles/kg, or about 400 nmoles/kg to about 600 nmoles/kg.

In various other embodiments, the dose of the one or more adaptor compounds, or the pharmaceutically acceptable salts thereof, or the rejuvenating compound, or the pharmaceutically acceptable salt thereof, or the immunosuppressive compound, or the pharmaceutically acceptable salt thereof, or the activity modifying compound, or the pharmaceutically acceptable salt thereof, may range from, for example, about 1 nmoles/kg to about 10,000 nmoles/kg, from about 1 nmoles/kg to about 5000 nmoles/kg, from about 1 nmoles/kg to about 3000 nmoles/kg, about 1 nmoles/kg to about 2500 nmoles/kg, about 1 nmoles/kg to about 2000 nmoles/kg, about 1 nmoles/kg to about 1000 nmoles/kg, about 1 nmoles/kg to about 900 nmoles/kg, about 1 nmoles/kg to about 800 nmoles/kg, about 1 nmoles/kg to about 700 nmoles/kg, about 1 nmoles/kg to about 600 nmoles/kg, about 1 nmoles/kg to about 500 nmoles/kg, about 1 nmoles/kg to about 400 nmoles/kg, about 1 nmoles/kg to about 300 nmoles/kg, about 1 nmoles/kg to about 200 nmoles/kg, about 1 nmoles/kg to about 150 nmoles/kg, about 1 nmoles/kg to about 100 nmoles/kg, about 1 nmoles/kg to about 90 nmoles/kg, about 1 nmoles/kg to about 80 nmoles/kg, about 1 nmoles/kg to about 70 nmoles/kg, about 1 nmoles/kg to about 60 nmoles/kg, about 1 nmoles/kg to about 50 nmoles/kg, about 1 nmoles/kg to about 40 nmoles/kg, about 1 nmoles/kg to about 30 nmoles/kg, or about 1 nmoles/kg to about 20 nmoles/kg.

In another embodiment, from about 20 μg/kg body weight to about 3 mg/kg body weight of the adaptor compound, or the pharmaceutically acceptable salt thereof, or the rejuvenating compound, or the pharmaceutically acceptable salt thereof, or the immunosuppressive compound, or the pharmaceutically acceptable salt thereof, or the activity modifying compound, or the pharmaceutically acceptable salt thereof, can be administered to the subject. In another aspect, amounts can be from about 0.2 mg/kg body weight to about 0.4 mg/kg body weight or can be about 50 μg/kg body weight.

Unless otherwise specified, in all the dosage embodiments set forth herein, “kg” is kilograms of body weight of the subject.

A single dose or multiple doses of the one or more adaptor compound(s), or the pharmaceutically acceptable salt thereof, or the rejuvenating compound, or the pharmaceutically acceptable salt thereof, or the immunosuppressive compound, or the pharmaceutically acceptable salt thereof, or the activity modifying compound, or the pharmaceutically acceptable salt thereof, can be administered to the subject.

The timing between the administration of CAR-T cells and the small molecule linked to the first targeting moiety (i.e., the adaptor compound, or the pharmaceutically acceptable salt thereof) can vary widely depending on factors that include the type of CAR-T cells being used, the binding specificity of the CAR, the identity of the first targeting moiety and the small molecule ligand, the identity of the cancer, the location in the subject of the cancer, the means used to administer to the subject the CAR-T cells and the adaptor compound, or the pharmaceutically acceptable salt thereof, and the health, age, and weight of the subject.

In at least one embodiment, the small molecule ligand linked to the first targeting moiety (i.e., the adaptor compound, or the pharmaceutically acceptable salt thereof) can be administered before or after the CAR-T cells (or composition thereof), such as within about 3, 6, 9, 12, 15, 18, 21, 24, 27, 30, 33, 36, 39, 42, 45, 48, or 51 hours, or within about 0.5, 1, 1.5, 2, 2.5, 3, 4 5, 6, 7, 8, 9, 10 or more days. In another embodiment, the one or more adaptor compounds, or pharmaceutically acceptable salts thereof, can be administered to the subject at the same time as the CAR-T cell composition, but in different formulations, or in the same formulation.

Any applicable dosing schedule known in the art can be used for administration of the adaptor compound(s), or the pharmaceutically acceptable salt thereof, the activity modifying compound, or the pharmaceutically acceptable salt thereof (e.g., the rejuvenating compound, or the pharmaceutically acceptable salt thereof, or the immunosuppressive compound, or the pharmaceutically acceptable salt thereof), or for the CAR-T cell composition. For example, once per day dosing (a.k.a qd), twice per day dosing (a.k.a. bid), three times per day dosing (a.k.a. tid), twice per week dosing (a.k.a. BIW), three times per week dosing (a.k.a. TIW), once weekly dosing, and the like, can be used. In one aspect, the dosing schedule selected can take into consideration the concentration of the compounds/compositions being administered (including, for example, the number of CAR-T cells administered) to regulate the cytotoxicity of the CAR-T cell composition and to control any potential adverse effects (e.g., CRS).

A method of facilitating treatment of cancer and/or killing cancer cells by way of modifying the activity of CAR-T cells in a subject, comprising administering the system of any of the foregoing embodiments to the subject, is also provided.

In some embodiments, a method of treating a cancer is provided using any of the compounds and compositions set forth herein. In the methods described herein, the cancer can additionally be imaged prior to administration to the subject of the adaptor compound(s), or the pharmaceutically acceptable salts thereof, or the CAR-T cell composition. For example, imaging can occur by positron emission tomography (PET) imaging, magnetic resonance imaging (MRI), or single-photon-emission computed tomography (SPECT)/computed tomography (CT) imaging. The imaging method can be any suitable imaging method known in the art. In one embodiment, the imaging method can involve the use of the small molecule ligand described herein (e.g., of the adaptor compound or salt thereof), but linked to an imaging agent suitable for the types of imaging described herein.

In at least one embodiment, a method of modifying the activity of CAR-T cells (e.g., treating a cancer) comprises administering to a subject a composition comprising CAR-T cells comprising a CAR directed to a first targeting moiety, a second targeting moiety, or both the first and second targeting moieties; administering to the subject one or more adaptor compounds, or pharmaceutically acceptable salts thereof, each adaptor compound comprising a small molecule ligand linked to the first targeting moiety; and administering to the subject an activity modifying compound linked to the second targeting moiety.

In certain embodiments, a method of modifying the activity of CAR-T cells and/or treating a cancer comprises administering to a subject a composition comprising a vector comprising a promoter operatively linked to a nucleic acid sequence encoding a CAR directed to a first targeting moiety, a second targeting moiety or both the first and second targeting moieties, wherein prior to, during, or after the administering step the subject received or receives a dose of one or more adaptor compounds, or pharmaceutically acceptable salts thereof, each adaptor compound comprising a small molecule ligand linked to the first targeting moiety. Additionally, in some embodiments, prior to, during, or after vector composition the administering step, the subject received or receives a dose of an activity modifying compound linked to the second targeting moiety.

Such vector composition can comprise the vector (e.g., lentiviral particles comprising the vector) comprising the nucleic acid vector that encodes at least a CAR described herein. In some embodiments, the vector composition comprises a therapeutically effective amount of the lentiviral particles according to any of the foregoing embodiments.

The vector composition can be administered by any route, including oral, nasal, intravenous, intraarterial, intramuscularly, or intraperitoneal routes. In some cases, the vector composition is administered by intravenous injection or by intratumoral injection.

Each of the one or more adaptor compounds, or pharmaceutically acceptable salts thereof, can be linked to the first targeting moiety via a first linker, and the activity modifying compound can be linked to the second targeting moiety via a second linker. The first and second linkers can comprise any of the linkers described herein and can have the same or different structures. Similarly, the first and second targeting moieties can comprise any of the targeting moieties described herein and can have the same or different structures. In another embodiment, the first and second targeting moieties have the same structure, while the first and second linkers have the same or different structures. Additionally, the activity modifying compound can comprise any of the activity modifying compounds described herein including, for example, a rejuvenating compound or an immunosuppressive compound.

“Cancer” has its plain and ordinary meaning when read in light of the specification and can include, but is not limited to, a group of diseases involving abnormal cell growth with the potential to invade or spread to other parts of the body. Numerous types of cancers can be treated using the compositions, compounds, and methods described herein including, without limitation, a carcinoma, a sarcoma, an osteosarcoma, a lymphoma, a melanoma, a mesothelioma, a nasopharyngeal carcinoma, a leukemia, an adenocarcinoma, and a myeloma. Other, and perhaps more specific, examples of cancers that can be treated in accordance with the methods and/or using the compounds and compositions hereof include, but are not limited to, lung cancer (including, without limitation, non-small cell lung cancer), bone cancer (including, without limitation, osteosarcoma), pancreatic cancer, skin cancer (including, without limitation, cutaneous melanoma), cancer of the head, cancer of the neck, intraocular melanoma, uterine cancer, ovarian cancer, endometrial cancer, rectal cancer, stomach cancer, colon cancer, breast cancer, triple negative breast cancer, carcinoma of the fallopian tubes, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, prostate cancer, leukemia (including, without limitation, chronic leukemia, acute leukemia, acute myelocytic leukemia, a lymphocytic lymphoma, myeloid leukemia, myelomonocytic leukemia, and hairy cell leukemia), pleural mesothelioma, cancer of the bladder, Burkitt's lymphoma, cancer of the ureter, cancer of the kidney (including, without limitation, renal cell carcinoma), carcinoma of the renal pelvis, a neoplasm of the central nervous system (CNS), primary CNS lymphoma, a spinal axis tumor, a brain stem glioma, a pituitary adenoma, and an adenocarcinoma of the gastroesophageal junction.

In some aspects of these embodiments, the cancer is a folate receptor expressing cancer, for example and without limitation, a folate receptor α-expressing cancer. In other embodiments, the cancer is a folate receptor β-expressing cancer. In some aspects of these embodiments, the cancer is an endometrial cancer, a non-small cell lung cancer, an ovarian cancer, or a triple-negative breast cancer.

The cancer being treated can be a tumor. In another embodiment, the cancer can be malignant. In another embodiment, the cancer is acute myelocytic leukemia such as, for example, an acute myelocytic leukemia where the cancer expresses folate receptor-β.

Certain embodiments of a method for modifying the activity of CAR-T cells and/or treating a cancer comprise: administering to a subject a composition comprising CAR-T cells, wherein the CAR-T cells comprise a CAR directed to a first targeting moiety, a second targeting moiety, or both the first and second targeting moieties; and administering to the subject one or more adaptor compounds, or pharmaceutically acceptable salts thereof, wherein each adaptor compound or pharmaceutically acceptable salt thereof comprises a small molecule ligand linked to the first targeting moiety via a first linker, wherein the first linker comprises a structure having the formula:

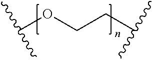

wherein n is an integer from 0 to 200.

In at least one embodiment, the method can additionally comprise administering to the subject a rejuvenating compound, or a pharmaceutically acceptable salt thereof, linked to the second targeting moiety (e.g., via a second linker or directly). In such embodiments, the rejuvenating compound, or the pharmaceutically acceptable salt thereof, can, for example, be selected from a group comprising a TLR agonist (e.g., agonists of TLR1, TLR2, TLR3, TLR4, TLR7, TLR8, TLR7/8, TLR9, etc.), a STING agonist, a NLR, an ALR agonist, a kinase inhibitor targeting kinase, a RLR, a RAGE, a phosphatase inhibitor, and any other pattern recognition receptor that is located in the endosome or cytoplasm of the targeted cell.

Additionally, the second linker can comprise a structure having the formula:

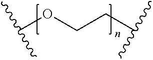

wherein n is an integer from 0 to 200.

In at least one examplary embodiment of a method for treating cancer, the rejuvenating compound, or the pharmaceutically acceptable salt thereof, is a TLR agonist (e.g., TLR 7, TLR7/8, or TLR8) having a structure of one of the following formulae:

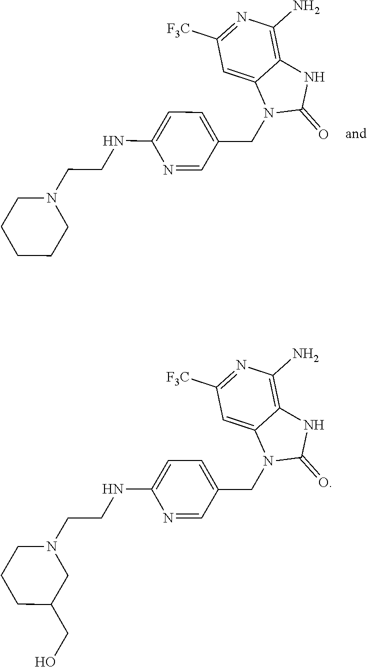 [SMILES: Nc1nc(C(F)(F)F)cc2c1[nH]c(=O)n2Cc1ccc(NCCN2CCCCC2)nc1 ; Nc1nc(C(F)(F)F)cc2c1[nH]c(=O)n2Cc1ccc(NCCN2CCCC(CO)C2)nc1]

In certain other embodiments, the rejuvenating compound or pharmaceutically acceptable salt thereof has the formula:

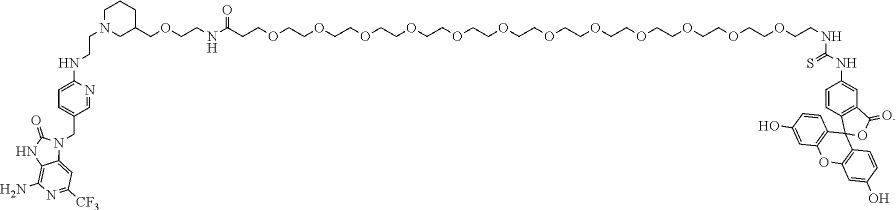 [SMILES: Nc1nc(C(F)(F)F)cc2c1[nH]c(=O)n2Cc1ccc(NCCN2CCCC(COCCNC(=O)CCOCCOCCOCCOCCOCCOCCOCCOCCOCCOCCOCCOCCNC(=S)Nc3ccc4c(c3)C(=O)OC43c4ccc(O)cc4Oc4cc(O)ccc43)C2)nc1]

In other embodiments of such methods, the rejuvenating compound has a structure of one of the following formulae:

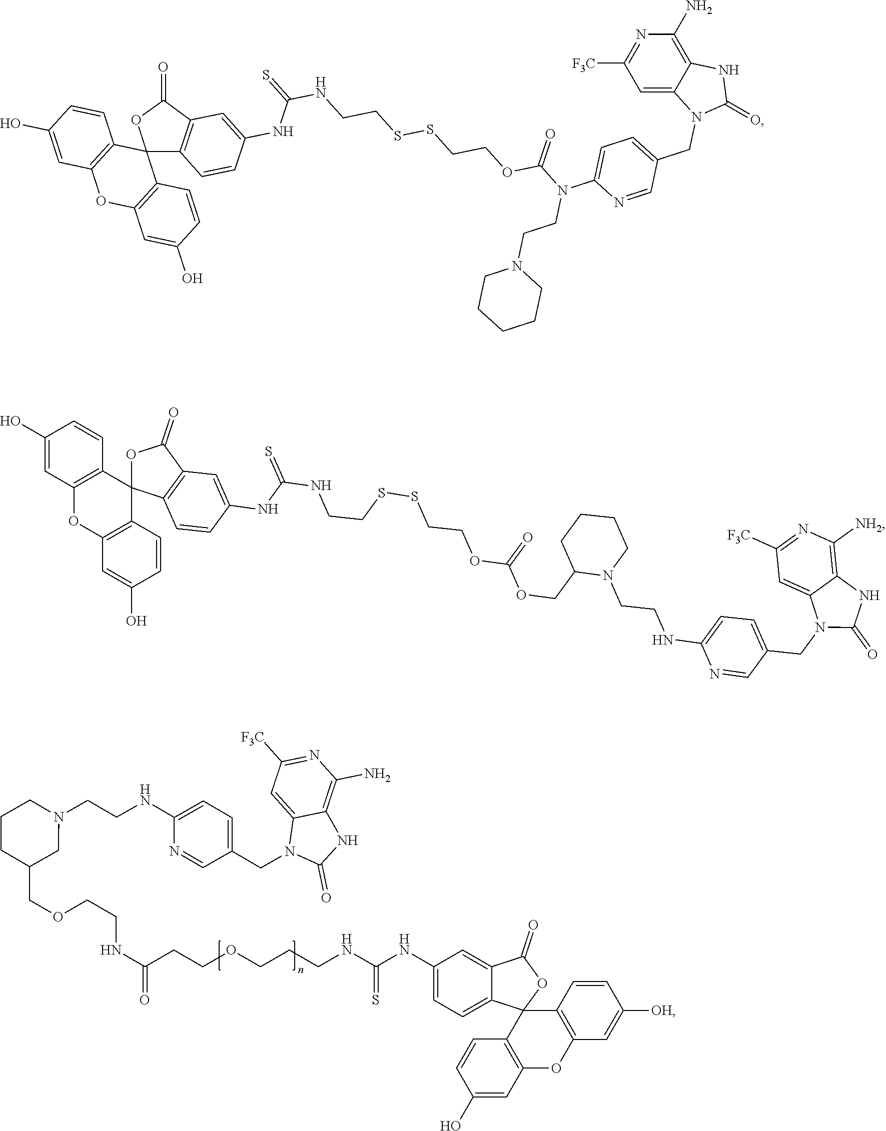

wherein n=0 to 200, and

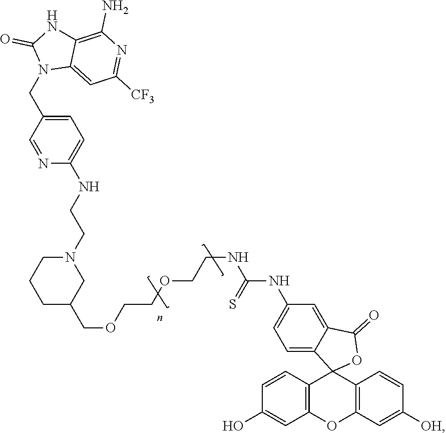

wherein n=0 to 50.

Methods of treating a subject having received CAR-T cell therapy are also provided. In certain embodiments, such methods comprise administering to the subject one or more adaptor compounds, or pharmaceutically acceptable salts thereof, each adaptor compound or pharmaceutically acceptable salt thereof comprising a small molecule ligand linked to a first targeting moiety; wherein, prior to the administering step, the subject has received a dose of CAR-T cells expressing a CAR that recognizes and binds to the first targeting moiety. In some embodiments, the method can further comprise administering to the subject an activity modifying compound linked to a second targeting moiety. The CAR-T cells can express one or more of the embodiments of CARs described herein (e.g., a CAR that recognizes and binds to the first targeting moiety, the second targeting moiety, or both the first and second targeting moieties). The one or more adaptor compounds, or pharmaceutically acceptable salts thereof, and the activity modifying compound of such methods can comprise any of the embodiments described herein.

In any of the embodiments described herein, cytokine release resulting in off-target toxicity in the subject may not occur even though CAR-T cell toxicity to the cancer occurs. In any embodiment described herein, off-target tissue toxicity may not occur in the subject even though CAR-T cell toxicity to the cancer occurs. In any embodiment described herein, the cancer may comprise a tumor, and tumor size may be reduced in the subject, even though off-target toxicity does not occur.

In any of the embodiments described herein, CRS can be reduced or prevented and the method can result in a decrease in tumor volume in the subject. In any embodiment described herein, body weight loss due to CRS can be reduced or prevented. In any embodiment described herein, the cancer can comprise a tumor and a complete response for the tumor can be obtained.

In the compounds, compositions, combinations, and methods, all embodiments of the adaptor compound, or a pharmaceutically acceptable salt thereof, the activity modifying compound, the CAR-T cell compositions, and the vector compositions are applicable, including, but not limited to, the targeting moiety embodiments and the linker embodiments.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

In the above description, numerous specific details are set forth to provide a thorough understanding of the present disclosure. Particular examples may be implemented without some or all of these specific details and it is to be understood that this disclosure is not limited to particular biological systems, particular cancers, or particular organs or tissues, which can, of course, vary but remain applicable in view of the data provided herein.

Additionally, various techniques and mechanisms of the present disclosure sometimes describe a connection or link between two components. Words such as attached, linked, coupled, connected, and similar terms with their inflectional morphemes are used interchangeably, unless the difference is noted or made otherwise clear from the context. These words and expressions do not necessarily signify direct connections but include connections through mediate components. It should be noted that a connection between two components does not necessarily mean a direct, unimpeded connection, as a variety of other components may reside between the two components of note. Consequently, a connection does not necessarily mean a direct, unimpeded connection unless otherwise noted.

Further, will be understood that the disclosure is presented in this manner merely for explanatory purposes and the principles and embodiments described herein may be applied to compounds and/or composition components that have configurations other than as specifically described herein. Indeed, it is expressly contemplated that the components of the composition and compounds of the present disclosure may be tailored in furtherance of the desired application thereof.

Unless defined otherwise, all technical and scientific terms used herein have the same meaning as commonly understood by one of skill in the chemical and biological arts. Although any methods and materials similar to or equivalent to those described herein can be used in the practice or testing of the subject of the present application, the preferred methods and materials are described herein. Additionally, as used in this specification and the appended claims, the singular forms “a”, “an” and “the” include plural referents unless the content clearly dictates otherwise. Thus, for example, where a compound/composition is substituted with “an” alkyl or aryl, the compound/composition is optionally substituted with at least one alkyl and/or at least one aryl.

When ranges are used herein for physical properties, such as molecular weight, or chemical properties, such as chemical formulae, all combinations and sub-combinations of ranges and specific embodiments therein are intended to be included.

Additionally, the term “about,” when referring to a number or a numerical value or range (including, for example, whole numbers, fractions, and percentages), means that the number or numerical range referred to is an approximation within experimental variability (or within statistical experimental error) and thus the numerical value or range can vary between 1% and 15% of the stated number or numerical range (e.g., +/−5% to 15% of the recited value) provided that one of ordinary skill in the art would consider equivalent to the recited value (e.g., having the same function or result). The term “comprising” (and related terms such as “comprise” or “comprises” or “having” or “including”) is not intended to exclude that in other certain embodiments, for example, an embodiment of any compound, composition of matter, composition, method, or process, or the like, described herein, may “consist of” or “consist essentially of” the described features. The term “substantially” can allow for a degree of variability in a value or range, for example, within 90%, within 95%, or within 99% of a stated value or of a stated limit of a range.

Where a method of therapy comprises administering more than one treatment, compound, or composition to a subject, it will be understood that the order, timing, number, concentration, and volume of the administration is limited only by the medical requirements and limitations of the treatment (i.e. two treatments can be administered to the subject, e.g., simultaneously, consecutively, sequentially, alternatively, or according to any other regimen).

Additionally, in describing representative embodiments, the disclosure may have presented a method and/or process as a particular sequence of steps. To the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present disclosure.

It is therefore intended that this description and the appended claims will encompass, all modifications and changes apparent to those of ordinary skill in the art based on this disclosure.

### EXAMPLES

The following examples illustrate certain specific embodiments of the present disclosure and are not meant to limit the scope of the claimed invention in any way.

### Example 1

### Synthesis of FITC-Folate

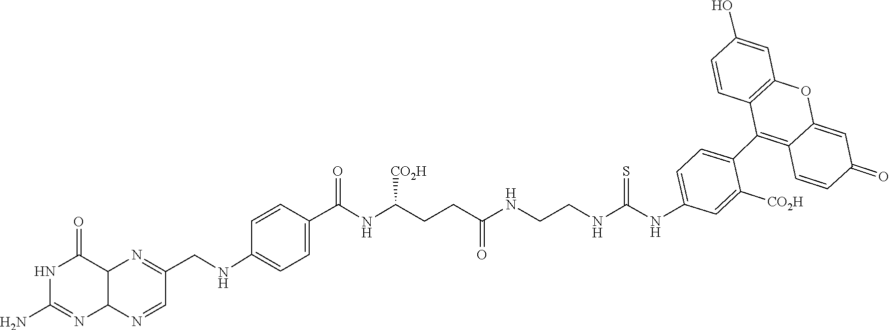 [SMILES: NC1=NC2N=CC(CNc3ccc(C(=O)N[C@@H](CCC(=O)NCCNC(=S)Nc4ccc(-c5c6ccc(=O)cc-6oc6cc(O)ccc56)c(C(=O)O)c4)C(=O)O)cc3)=NC2C(=O)N1]

Folate-γ-ethylenediamine was coupled to fluorescein isothiocyanate (FITC) isomer I (Sigma-Aldrich) in anhydrous dimethylsulfoxide (DMF) in the presence of tetramethylguanidine and diisopropylamine. The crude product was loaded onto an Xterra RP18 preparative HPLC column (Waters) and eluted with gradient conditions starting with 99% 5 mM sodium phosphate (mobile phase A, pH 7.4) and 1% acetonitrile (mobile phase B) and reaching 90% A and 10% B in 10 min at a flow rate of 20 mL/min.

Under these conditions, the FITC-folate main peak typically eluted at 27-50 min. The quality of the FITC-folate fraction was monitored by analytical reverse-phase HPLC with a UV detector. Fractions with greater than 98.0% purity (LCMS) were lyophilized to obtain the final FITC-folate product. As known in the art, the compound with this structure is also referred to as EC17.

### Example 2

### Synthesis of FITC-PEG12-Folate

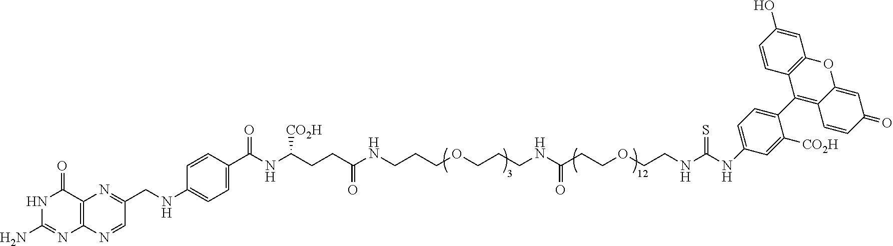

Universal polyethylene glycol (PEG) Nova Tag™ resin (0.2 g) was loaded into a peptide synthesis vessel and washed with isopropyl alcohol (i-PrOH) (3×10 mL) and dimethylformamide (DMF, 3×10mL). 9-fluorenylmethoxycarbonyl (Fmoc) deprotection was carried out using 20% piperidine in DMF (3×10 mL). Kaiser tests were performed to assess reaction progress. A solution of Fmoc-L-glutamic acid 5-tert-butyl ester (Fmoc-Glu-(O-t-Bu)-OH) (23.5 mg) in DMF, N,N-diisopropylethylamine (i-Pr₂NEt) (4 equiv), and benzotriazol-1-yl-oxytripyrrolidinophosphonium hexafluorophosphate (PyBOP) (2 equiv) was then introduced to the vessel. Fmoc deprotection was carried out using 20% piperidine in DMF (3×10 mL). A solution of N¹⁰-TFA-Pte-OH (22.5 mg), DMF, i-Pr₂NEt (4 equiv), and PyBOP (2 equiv) was then introduced to the vessel. Argon was bubbled for 2 h, and the resin was washed with DMF (3×3 mL) and i-PrOH (3×3 mL). After swelling the resin in dichloromethane (DCM), a solution of 1M hydroxybenzotriazole (HOBT) in DCM/trifluoroethane (TFE) (1:1) (2×3 mL) was added. Argon was bubbled for 1 h, the solvent was removed, and the resin was washed with DMF (3×3 mL) and i-PrOH (3×3 mL). After swelling the resin in DMF, a solution of Fmoc-NH-(PEG)₁₂-COOH (46.3 mg) in DMF, i-Pr₂NEt (4 equiv), and PyBOP (2 equiv) was added. Argon was bubbled for 2 h, and the resin was washed with DMF (3×3 mL) and i-PrOH (3×3 mL).

Fmoc deprotection was carried out using 20% piperidine in DMF (3×10 mL). Kaiser tests were performed to assess reaction progress.

A solution of FITC (Life Technologies 21.4 mg) in DMF and i-Pr₂NEt (4 equiv) was then introduced to the vessel, then Argon was bubbled for 2 h, and the resin was washed with DMF (3×3 mL) and i-PrOH (3×3 mL). Then to the vessel was added 2% NH₂NH₂ in DMF (2×2 mL). The final compound was cleaved from the resin using a TFA:H₂O: triisopropylsilane (TIS) (95:2.5:2.5) (Cleavage Solution) and concentrated under vacuum. The concentrated product was precipitated in Et₂O and dried under vacuum. The crude product was purified using preparative RP-HPLC (mobile phase: A=10 mM ammonium acetate pH=7, B=ACN; method: 0% B to 30% B in 30 min at 13 mL/min). The pure fractions were pooled and freeze-dried, providing the FITC-PEG12-Folate.

### Example 3

### Synthesis of FITC-PEG20-Folate

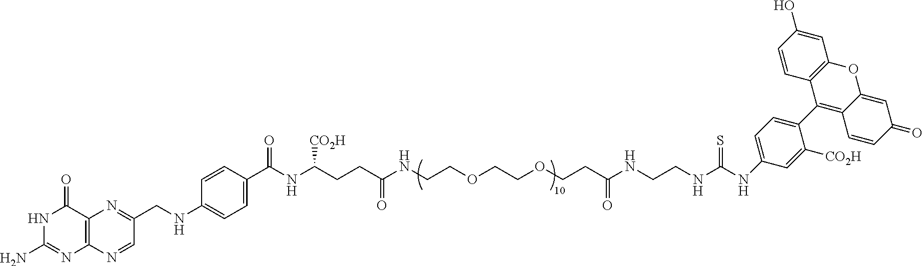

Ethylenediamine, polymer-bound (200-400 mesh)-resin (50 mg) was loaded into a peptide synthesis vessel and swollen with DCM (3 mL) followed by DMF (3 mL). To the vessel was then introduced the Fmoc-PEG₂₀-COOH solution (131 mg, 1.0 equiv) in DMF, i-Pr₂NEt (6.0 equiv), and PyBOP (4.0 equiv). Argon was bubbled for 6 h, the coupling solution was drained, and the resin was washed with DMF (3×10 mL) and i-PrOH (3×10 mL). Kaiser tests were performed to assess reaction progress. Fmoc deprotection was carried out using 20% piperidine in DMF (3×10 mL), before each amino acid coupling. The above sequence was repeated to complete the reaction with Fmoc-Glu-OtBu (72 mg, 2.0 equiv) and Tfa.Pteroic-acid (41 mg, 1.2 equiv) coupling steps. The resin was washed with 2% hydrazine in DMF 3×10 mL (5 min) to cleave the trifluoro-acetyl protecting group on pteroic acid and washed with i-PrOH (3×10 mL) followed by DMF (3×10mL). The resin was dried under argon for 30 min. The folate-peptide was cleaved from the resin using the Cleavage Solution. 10 mL of the cleavage mixture was introduced, and argon was bubbled for 1.5 h. The cleavage mixture was drained into a clean flask. The resin was washed 3 times with more cleavage mixture. The combined mixture was concentrated under reduced pressure to a smaller volume (˜5 mL) and precipitated in ethyl ether.

The precipitate was collected by centrifugation, washed with ethyl ether (3 times) and dried under high vacuum. The dried Folate-PEG₂₀-EDA (1.0 equiv) was treated with FITC (50 mg, 1.5 equiv) in DMSO and DIPEA at room temperature. Progress of the reaction monitored by LCMS. After 8 h the starting material was consumed to give the product. The crude reaction mixture was purified by preparative HPLC, (mobile phase A=10 mM Ammonium Acetate, pH=7; Organic phase B=Acetonitrile; Method: 0% B to 30% B in 35 minutes at 13 mL/min) and provided FITC-PEG20-Folate in 60% yield.

### Example 4

### Synthesis of FITC-PEG108-Folate

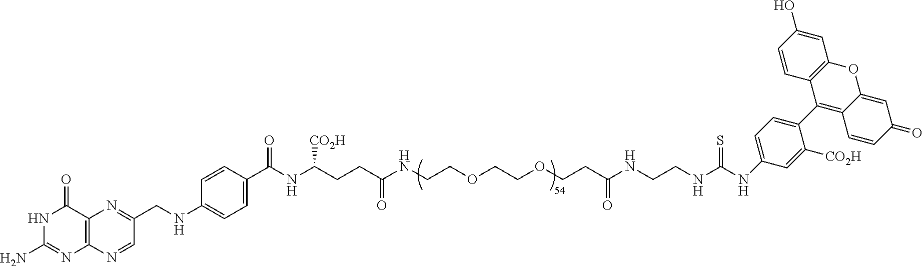

Ethylenediamine, polymer-bound (200-400 mesh)-resin (50 mg) was loaded in a peptide synthesis vessel and swollen with DCM (3 mL) followed by DMF (3 mL). To the vessel was then introduced the Fmoc-PEG₃₆-COOH solution (161 mg, 1.0 equiv) in DMF, i-Pr₂NEt (6.0 equiv), and PyBOP (4.0 equiv). Argon was bubbled for 6 h, the coupling solution was drained, and the resin was washed with DMF (3×10 mL) and i-PrOH (3×10 mL). Kaiser tests were performed to assess reaction progress. Fmoc deprotection was carried out using 20% piperidine in DMF (3×10 mL), before each amino acid coupling. The above sequence was repeated to complete reaction with 2× Fmoc-PEG₃₆-COOH (161 mg, 1.0 equiv), Fmoc-Glu-OtBu (72 mg, 2.0 equiv) and Tfa.Pteroic-acid (41.0 mg, 1.2 equiv) coupling steps. At the end the resin was washed with 2% hydrazine in DMF 3×10mL (5 min) to cleave the trifluoro-acetyl protecting group on pteroic acid and washed with i-PrOH (3×10mL) followed by DMF (3×10mL). The resin was dried under argon for 30 min. Folate-peptide was cleaved from the resin using the Cleavage Solution. 10 mL of the cleavage mixture was introduced and argon was bubbled for 1.5 h. The cleavage mixture was drained into a clean flask. The resin was washed 3× with more Cleavage Solution. The combined mixture was concentrated under reduced pressure to a smaller volume (˜5 mL) and precipitated in ethyl ether.

The precipitate was collected by centrifugation, washed with ethyl ether (3×) and dried under high vacuum. The dried Folate-PEG₁₀₈-EDA (1.0 equiv) was treated with FITC (50 mg, 1.5 equiv) in DMSO and DIPEA at room temperature. Reaction progress was monitored by LCMS. After 10 h starting material was consumed to give the product. The crude reaction mixture was purified by preparative HPLC, (mobile phase A=10 mM Ammonium Acetate, pH=7; Organic phase B=Acetonitrile; Method: 0% B to 30% B in 35 minutes at 13 mL/min) and provided FITC-PEG108-Folate in 64% yield.

Example 5

### Synthesis of FITC-DUPA

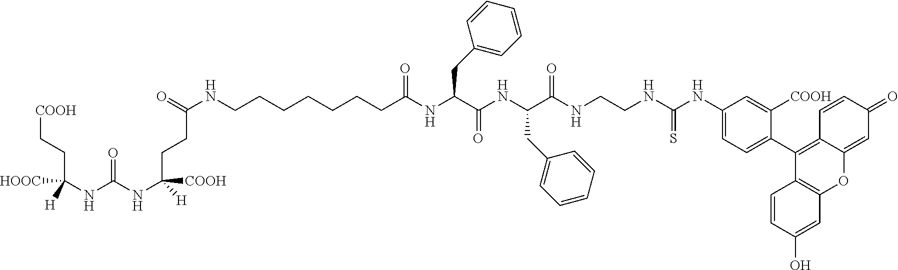 [SMILES: O=C(O)CC[C@H](NC(=O)N[C@@H](CCC(=O)NCCCCCCCC(=O)N[C@@H](Cc1ccccc1)C(=O)N[C@@H](Cc1ccccc1)C(=O)NCCNC(=S)Nc1ccc(-c2c3ccc(=O)cc-3oc3cc(O)ccc23)c(C(=O)O)c1)C(=O)O)C(=O)O]

DUPA-FITC was synthesized by solid phase methodology as follows. Universal Nova Tag™ resin (50 mg, 0.53 mM) was swollen with DCM (3 mL) followed by DMF 3 mL). A solution of 20% piperidine in DMF (3×3 mL) was added to the resin, and argon was bubbled for 5 min. The resin was washed with DMF (3×3 mL) and isopropyl alcohol (i-PrOH, 3×3 mL). After swelling the resin in DMF, a solution of DUPA-(OtBu)-OH (1.5 equiv), HATU (2.5 equiv), and i-Pr₂NEt (4.0 equiv) in DMF was added. Argon was bubbled for 2 h, and resin was washed with DMF (3×3 mL) and i-PrOH (3×3 mL).

After swelling the resin in DCM, a solution of 1 M HOBt in DCM/TFE (1:1) (2×3 mL) was added. Argon was bubbled for 1 h, the solvent was removed and resin was washed with DMF (3×3 mL) and i-PrOH (3×3 mL). After swelling the resin in DMF, a solution of Fmoc-Phe-OH (2.5 equiv), HATU (2.5 equiv) and DIPEA (4.0 equiv) in DMF was added. Argon was bubbled for 2 h, and the resin was washed with DMF (3×3 mL) and i-PrOH (3×3 mL).

The above sequence was repeated for 2 more coupling steps for addition of 8-aminooctanoic acid and fluorescein isothiocyanate or rhodamine B isothiocyanate.

The final compound was cleaved from the resin using the Cleavage Solution and concentrated under vacuum. The concentrated product was precipitated in diethyl ether and dried under vacuum. The crude product was purified using preparative RP-HPLC [λ=488 nm; solvent gradient: 1% B to 80% B in 25 min, 80% B wash 30 min run; A=10 mM NH₄OAc, pH=7; B=acetonitrile (ACN)]. ACN was removed under vacuum, and purified fractions were freeze-dried to yield FITC-DUPA as a brownish-orange solid. RP-HPLC: tR=8.0 min (A=10 mM NH₄OAc, pH=7.0; B=ACN, solvent gradient: 1% B to 50% B in 10 min, 80% B wash 15 min run). ¹H NMR (DMSO-d6/D₂O): δ0.98-1.27 (ms, 9H); 1.45 (b, 3H); 1.68-1.85 (ms, 11H); 2.03 (m, 8H); 2.6-3.44 (ms, 12H); 3.82 (b, 2H); 4.35 (m, 1H); 6.53 (d, J=8.1 Hz, 2H), 6.61 (dd, J=5.3, 3.5 Hz, 2H); 6.64 (s, 2H); 7.05 (d, J=8.2 Hz, 2H), 7.19 (m, 5H); 7.76 (d, J=8.0 Hz, 1H); 8.38 (s, 1H). HRMS (ESI) (m/z): (M+H)⁺ calculated for C₅₁H₅₉N₇O₁₅S, 1040.3712, found, 1040.3702. UV/vis: λ max=491 nm.

### Example 6

### Synthesis of FITC-PEG12-DUPA

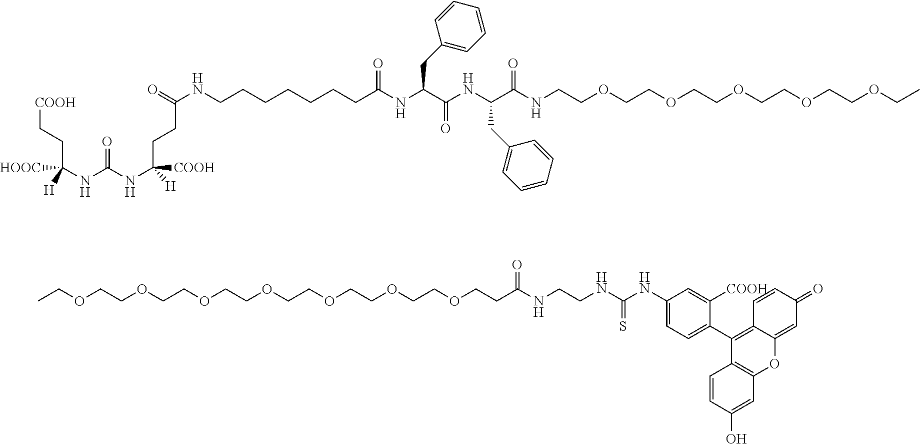

1,2-Diaminoethane trityl-resin (0.025 g) was loaded into a peptide synthesis vessel and washed with i-PrOH (3×10 mL), followed by DMF (3×10 mL). To the vessel was then introduced a solution of Fmoc-NH-(PEG)₁₂-COOH (42.8 mg) in DMF, i-Pr₂NEt (2.5 equiv), and PyBOP (2.5 equiv). The resulting solution was bubbled with Ar for 1 h, the coupling solution was drained, and the resin washed with DMF (3×10 mL) and i-PrOH (3×10 mL). Kaiser tests were performed to assess reaction progress. Fmoc deprotection was carried out using 20% piperidine in DMF (3×10 mL). This procedure was repeated to complete the all coupling steps (2×1.5 equiv of Fmoc-Phe-OH and 1.5 equiv of 8-aminooctanoic acid and 1.2 equiv of DUPA were used on each of their respective coupling steps).

After the DUPA coupling, the resin was washed with DMF (3×10 mL) and i-PrOH (3×10 mL) and dried under reduced pressure. The peptide was cleaved from the resin in the peptide synthesis vessel using the Cleavage Solution. 15 mL of the Cleavage Solution was added to the peptide synthesis vessel, and the reaction was bubbled under Ar for 15 min. The resin was treated with two additional 10 mL quantities of the Cleavage Solution for 5 min each. The cleavage mixture was concentrated to about 5 mL and precipitated with ethyl ether. The precipitate was collected by centrifugation, washed with ethyl ether (3×), and dried under high vacuum, resulting in the recovery of crude material.

To a stirred solution of the crude DUPA-(PEG)₁₂-EDA (10 mg) and FITC (5.6 mg) in dimethylsulfoxide (DMSO, 1 mL) was added i-Pr₂NEt (5 equiv) at room temperature and stirred for 6 h under argon. The reaction was monitored by LCMS and purified by preparative HPLC (mobile phase: A=10 mM ammonium acetate pH=7, B=ACN; method: 0% B to 50% B in 30 min at 13 mL/min). The purified fractions were pooled and freeze-dried, providing the FITC-PEG12-DUPA.

### Example 7

### Synthesis of FITC-PEG11-NK1

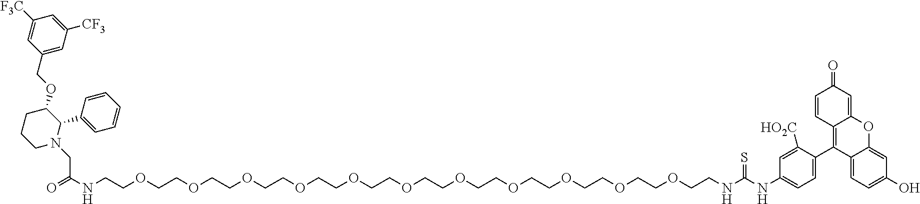 [SMILES: O=C(CN1CCC[C@H](OCc2cc(C(F)(F)F)cc(C(F)(F)F)c2)[C@@H]1c1ccccc1)NCCOCCOCCOCCOCCOCCOCCOCCOCCOCCOCCOCCNC(=S)Nc1ccc(-c2c3ccc(=O)cc-3oc3cc(O)ccc23)c(C(=O)O)c1]

To a stirred solution of NK-1 (0.02 g, 0.0433 mmol, 1.0 eq.), O-(2-Aminoethyl)-O′-[2-(Boc-amino)ethyl]decaethylene glycol (BocNH-PEG₁₁-NH₂) (Sigma, 0.0336 g, 0.0521 mmol, 1.2 eq.), Benzotriazol-1-yl-oxytripyrrolidinophosphonium hexafluorophosphate (PyBOP) (0.027 g, 0.0521 mmol, 1.2 eq.) in dry CH₂Cl₂ was added N,N-Diisopropylethylamine (DIPEA) (0.076 mL, 0.4338 mmol, 10 eq.) under argon at room temperature. The reaction progress was monitored by LCMS and purified by preparative RP-HPLC (Waters, XBridge™ Prep C18, 5 μm; 19×100 mm column, mobile phase A=20 mM ammonium acetate buffer, pH 7, B=acetonitrile, gradient 10-100% B in 30 min, 13 mL/min, λ=220 nm, 254 nm). The pure fractions were collected, all organic solvents were evaporated and the sample was lyophilized for 48 h to provide the NK1-PEG₁₁-NHBoc. Yield: 40.13 mg (97%). To the NK1-PEG₁₁-NHBoc (0.0165 g, 0.015 mmol) in dry DCM was added trifluoroacetic acid (TFA, 20 eq.) and the reaction mixture was stirred for 4 h at r.t. The excess TFA was removed, and the remaining solution was diluted with water and extracted using CH₂Cl₂ (3×5 mL). The combined organic layers were washed with brine, dried (Na₂SO₄) and concentrated. The residue obtained was dried under vacuum and used for the nextstep without further purification. A stirred solution of NK1-PEG₁₁-NH₂ (0.008 g, 0.0081 mmol, 1.0 eq.), Fluorescein isothiocyanate (FITC) (Sigma, 0.0037 g, 0.0097 mmol, 1.2 eq.) in dry dimethylsulfoxide (DMSO, 0.3 mL) was added to diisopropylethyl amine (0.0028 mL, 0.0162 mmol, 2.0 eq.) at room temperature under argon. The reaction progress was monitored by LCMS and the product was purified by preparative RP-HPLC (Waters, XBridge™ Prep C18, 5 μm; 19×100 mm column, mobile phase A=20 mM ammonium acetate buffer, pH 7, B=acetonitrile, gradient 10-100% B in 30 min, 13 mL/min, λ=280 nm). The pure fractions were collected, all organic solvents were evaporated and the sample was lyophilized for 48 h to provide the FITC-PEG11-NK1 in a yield of 8.54 mg (77%).

*Note: The NK-1 compound was synthesized by a two-step procedure starting from the base ligand, which was prepared by using a procedure in the literature. (Ref: DESIGN AND DEVELOPMENT OF NEUROKININ-1 RECEPTOR-BINDING AGENT DELIVERY CONJUGATES, Application Number: PCT/US2015/44229; incorporated herein by reference.

### Example 8

### Synthesis of FITC-PEG2-CA9

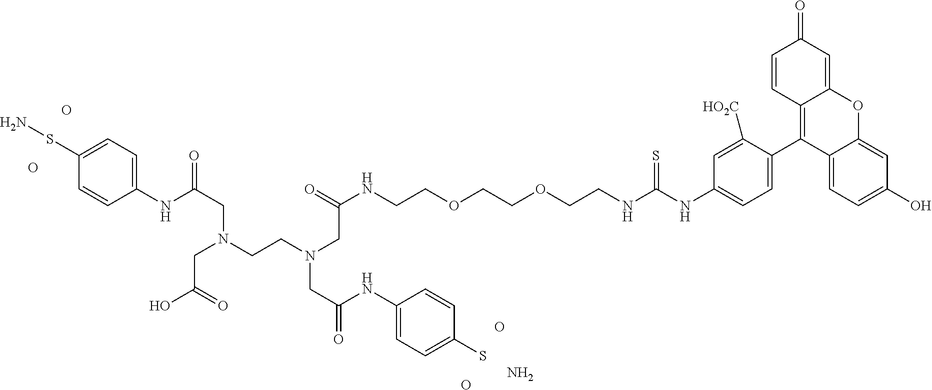

CA9 ligand (53.6 mg) was dissolved in DMF (2-3 mL) in a 50 mL round bottom flask using a Teflon magnetic stir bar. Ambient air was removed using a vacuum and replaced with nitrogen gas, this was done in three cycles. The round bottom flask was kept under constant nitrogen gas. To the flask, 28.9 mg of N-(3-Dimethylaminopropyl)-N′-ethylcarbodiimide hydrochloride (EDC) was added followed by 21.6 mg 1-Hydroxybenzotriazole hydrate (HOBt) and 18.9 μL of Boc-PEG₂-NH₂ (Sigma Aldrich). 5.4 μL of triethylamine (TEA) was added and the reaction was stirred overnight. The reaction mixture was purified using HPLC and confirmed with UHPLC-MS (target m/z of 831). Acetonitrile was removed using high vacuum rotary evaporation and the product lyophilized. The compound was mixed with 1:1 TFA:DCM for 30 minutes. The TFA/DCM was removed using high vacuum rotary evaporation followed by 30 minutes on high vacuum. The compound was then dissolved in DMF and combined with 5 molar equivalents of i-Pr₂NEt, 16 mg of fluorescein isothiocyanate (Life Technologies) and stirred for 1 h. The reaction mixture was purified by HPLC and the target compound was confirmed with UHPLC-MS (target m/z of 1120). The samples were lyophilized and stored at −20° C.

### Example 9

### T Cell Preparation

Human peripheral blood mononuclear cells (PBMCs) were isolated from whole blood of healthy donors by using Ficoll density gradient centrifugation (GE Healthcare Lifesciences). T cells were then isolated from PBMCs by using an EasySep™ Human T Cell Isolation Kit (STEM CELL technologies). T cells were cultured in TexMACS medium (Miltenyi Biotech Inc) with 40-100 IU/mL human IL-2 (Miltenyi Biotech), 2% human AB type serum, and 1% penicillin/streptomycin sulfate. Dynabeads Human T-Activator CD3/CD28 (ThermoFisher Scientific) were added to T cells at 1:1 ratio to activate T cells. 12-24 hours after activation, T cells were transduced with FITC-CAR lentiviral particles in the presence of 8 μg/mL polybrine (Santa Cruiz Biotech) by spinfection at 1,200 g for 90 minutes at 22-32° C.

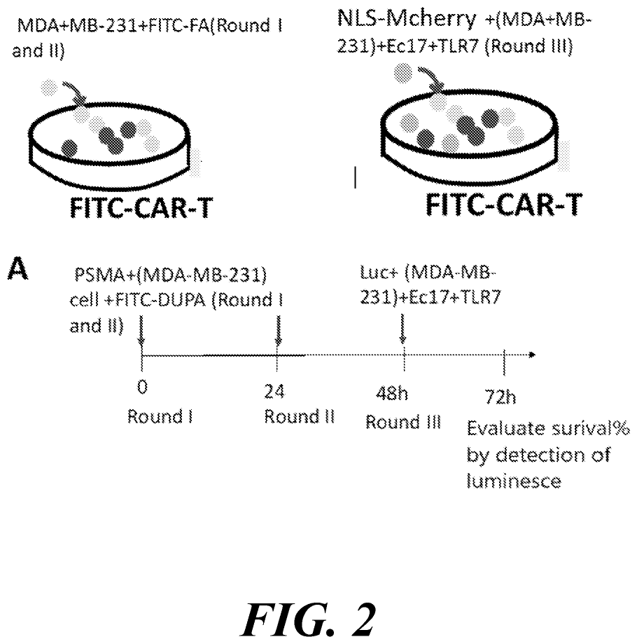

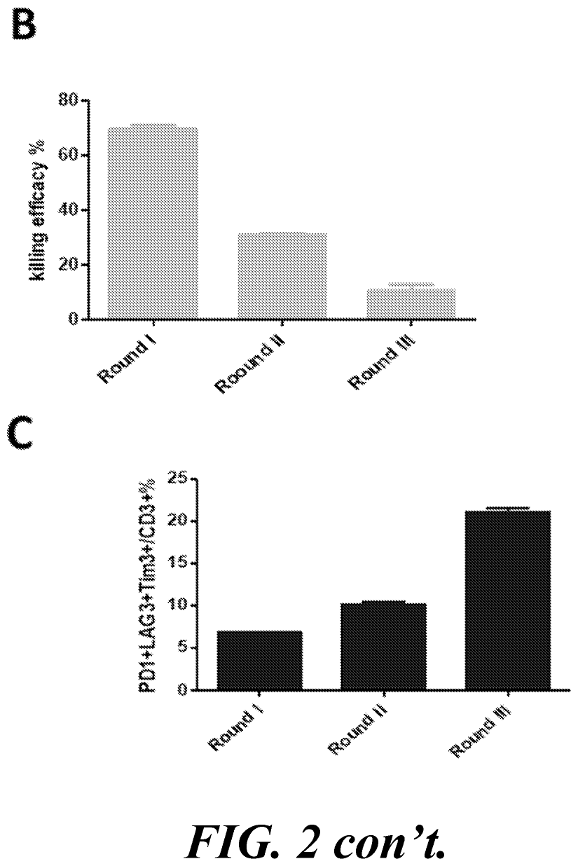

T cell mixture containing those with CAR modification (CAR-Ts) and those without CAR modification (non-transformed Ts) was cultured in the presence of activation beads for 6 days before the removal of activation beads. Fluorescence-Activated Cell Sorting was used to sort out CAR-T cells (GFP positive) and non-transformed T cells (GFP negative) based on their GFP expression. The sorted T cells were cultured for 7-15 days before injection into mice. When a T cell mixture was used, CAR-T cells and non-transformed T cells were mixed at the desired ratio before mouse injection. The data shown in FIGS. 2-4E was obtained with T cells prepared with these procedures.

### Example 10

### Generation of Lentiviral Vector Encoding CAR Gene

An overlap PCR method was used to generate CAR constructs comprising scFv against fluorescein. scFV against fluorescein, 4M5.3 (Kd=270 fM, 762 bp) derived from anti-fluorescein (4-4-20) antibody was synthesized. Sequence encoding the human CD8α signal peptide (SP, 63 bp), the hinge, and transmembrane region (249 bp), the cytoplasmic domain of 4-1BB (CD137, 141 bp) and the CD3ζ chain (336 bp), as shown in FIG. 1, were fused with the anti-fluorescein scFV by overlapping PCR. The resulting CAR construct (1551 bp) was inserted into EcoRI/NotI cleaved lentiviral expression vector pCDH-EF1-MCS-(PGK-GFP) (FIG. 1, System Biosciences). The sequence of the CAR construct in lentiviral vector was confirmed by DNA sequencing. Unless otherwise specified herein, the CAR construct used to generate the data for the Examples, has the nucleic acid sequence of SEQ ID NO: 1 and the amino acid sequence of SEQ ID NO: 2.

An exemplary CAR nucleic acid coding sequence can comprise SEQ ID NO: 1, wherein the first ATG is the start codon.

An exemplary CAR amino acid sequence can comprise SEQ ID NO: 2.

An exemplary insert can comprise SEQ ID NO: 3, wherein the first GCCACC sequence can comprise a restriction enzyme cleavage site, followed by the ATG start codon. In certain embodiments, the amino acid sequence encoded by SEQ ID NO: 1 or 3 can comprise SEQ ID NO: 2.

### Example 11

### Production of Lentivirus Containing CAR Gene for Human T Cell Transduction

To prepare lentiviral virus containing an anti-fluorescein (i.e., anti-FITC) single chain fragment variable (scFv) CAR, a HEK-293TN packaging cell line was co-transfected with the lentiviral vector encoding anti-fluorescein scFv CAR and a 2nd generation of a lentiviral packaging plasmid mix (Cellecta) or ViraPower Lentivrial Packaging Mix (ThermoFisher). After 24 and 48 hours of transfection, supernatants containing the lentivirus with the CAR gene were harvested and virus particles were concentrated by the standard polyethylene glycol virus concentration method (Clontech) for future transduction with human T cells.

### Example 12

### Isolation of Human T Cells from Human PBMC

T cells were isolated from human peripheral blood mononuclear cells (PBMC) by Ficoll density gradient centrifugation (GE Healthcare Lifesciences). After washing away remaining Ficoll solution, T cells were isolated by using an EasySep™ Human T Cell Isolation Kit (STEM CELL technologies). Purified T cells were cultured in TexMACS™ medium (Miltenyi Biotech Inc) with 1% penicillin and streptomycin sulfate in the presence of human IL-2 (100 IU/mL, Miltenyi Biotech Inc). T cells were cultured at density of 1×10⁶ cells/mL in multi-well plates. T cells were split and re-feed every 2-3 days.

### Example 13

### Transduction of Human T Cells

Isolated T cells were activated with Dynabeads coupled with anti-CD3/CD28 antibodies (Life Technologies) for 12-24 hours in the presence of human IL-2 (100 IU/mL), then transduced with lentivirus encoding an anti-fluorescein CAR gene. Cells were harvested after 72 hours and the expression of CAR on transduced T cells was identified by measuring GFP fluorescent cells using flow cytometry.

### Example 14

### CAR-T Cell Exhaustion Models (In Vitro and In Vivo)

For initial refinement of this strategy in vitro, exhausted CAR-T cells (10⁴/well) were generated by their continuous transfer (every 12 hours) to fresh MDA-MB-231 cells (10⁴/well) in presence of a FITC-folate adaptor compound while monitoring for appearance of exhaustion markers (PD-1⁺Tim3⁺LAG3⁺) and loss of CAR-T cell cytotoxicity. The abilities of both nontargeted and FITC-targeted TLR7 agonists to reverse the CAR-T cell exhaustion were then documented.

To evaluate whether the same FITC-TLR7 agonists might reverse exhaustion/dysfunction in vivo, NSG mice bearing KB tumors were treated with the same anti-FITC CAR-T cells until the above hallmarks of CAR-T cell dysfunction/exhaustion became prominent. Upon tail vein injection of the same FITC-TLR7 agonist, it was observed that the T cell exhaustion/dysfunction markers were reversed and tumor killing was restored.

For the in vitro model, more particularly, anti-FITC CAR-T cells (10⁴/well) were co-cultured with MDA-MB-231 (10⁴/well) at 1:1 ratio in a 96 well plate, while fresh MDA-MB-231 cell (10⁴/well) were added every 12 h to the wells two times to exhaust the FITC-CAR-T cells. For the treatment with the rejuvenating compound, after 2 rounds of stimulation with MDA-MB-231 cells, mcherry+ (MDA-MB-231; 10⁴/well) cells were added instead, and the rejuvenating compound was added at different concentrations. The cells were co-cultured with the exhausted anti-FITC CAR-T cells for 16 hours. The number of live mcherry+ (MDA-MB-231) cells were then counted by Incucyte S3 every 4 hours. The killing efficacy was calculated by dividing the numbers of cells at 16 h/numbers of cell at 8 h and multiplying ×100%.

For the in vivo model, more particularly, 8-10-week-old NSG mice (strain No. 005557) from Jackson Lab were used. All of the NSG mice were maintained on a folic acid-deficient diet (TD.95247, Envigo) in order to reduce the level of folic acid in mice to physiological levels found in humans. NSG mice were then implanted with 1.5×10⁶ KB cells into the flank. Once tumors reached around 30-50 mm³, all the mice were divided into two groups: a non-CAR-T cell treatment group and a CAR-T cell treatment group. The CAR-T cell treatment group received 1×10⁷ anti-FITC-CAR-T cells by i.v. injection. Four hours and 24 hours later, the CAR-T cell treatment group mice were given 500 nmol/kg of FITC-folic acid adaptor compound. Five days post CAR-T cell injection, all the CAR-T cell treatment group mice were divided into two subgroups: a saline vehicle control group and a TLR7 treatment group. The mice in the TLR7 treatment group received 10 nmol/mouse of FITC-TLR7 two times per week. Control groups received an equal volume of 100 μl of saline vehicle two times/week. All of the CAR-T cell treatment group mice continued to be given 500 nmol/kg of FITC-folic acid once a week. Tumor volume was measured unblinded with a caliper and was calculated using the formula (a×b²)/2 (a being the largest and b being the smallest diameter of the tumor).

### Example 15

### Flow Cytometry of CAR-T Cells Infiltrating Tumors

Tumor digestion was followed by use of the MACS kit (Cat#130-095-929). Single-cell suspensions were pre-incubated with Fc Blocker (anti-mouse CD16/CD32) for 10 minutes on ice followed by incubation with conjugated antibodies at 4° C. for 30 minutes in the dark. Cells were washed twice with FACS buffer prior to acquisition. FACS acquisition was performed using a Fortessa (BD) flow cytometer. Data were analyzed using Flowjo.

### Example 16

### Exhaustion of the Anti-FITC CAR-T Cells In Vitro

In this Example, an exhausted CAR-T cell model as described above was used. Briefly, FIG. 2B shows the results of an assay where MDA-MB-231 cells were co-cultured with anti-FITC CAR-T cells and FITC-folic acid adaptor compound, and fresh MDA-MB-231 cells were added to the co-culture every 12 hours 3 times consecutively. The results in FIG. 2B shows that these CAR-T cells became exhausted after stimulation 2 times by MDA-MB-231 cells in vitro, indicated by the decreased killing efficacy, and increased exhaustion markers expression (PD-1+Tim3+Lag3+).

### Example 17

### Synthesis of FITC-TLR7 Agonist Rejuvenating Compounds with Releasable or Non-Releasable Linkers

In this Example, the synthesis of FITC-TLR7 agonist rejuvenating compounds with releasable or non-releasable linkers is shown.

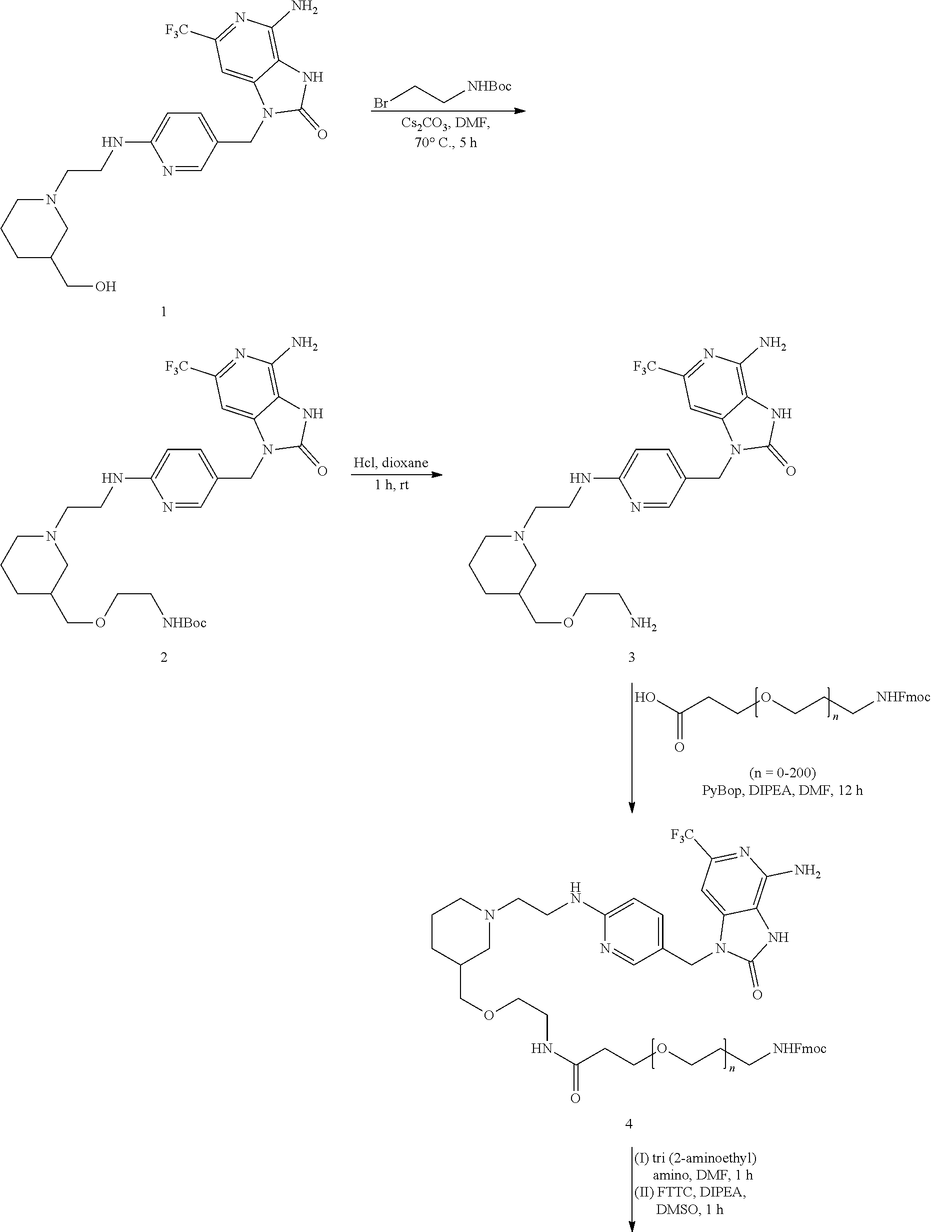

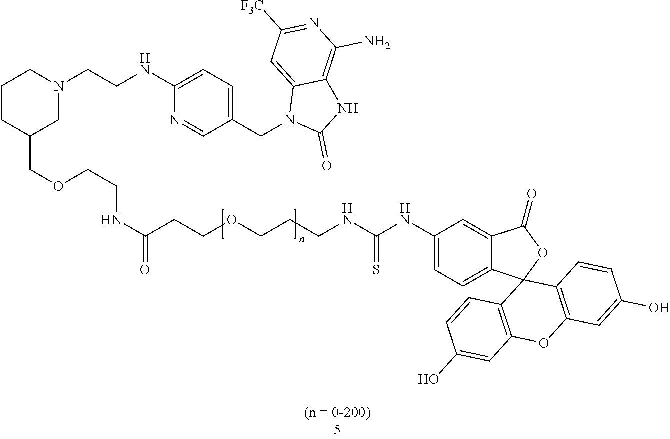

To a stirred solution of compound 1 (1 equiv) in DMF, Cs₂CO₃ (2 equiv) and Boc-bromoethyl amine (1.5 equiv) was added and heated at 70° C. for 5 h. Once the starting materials were consumed, the reaction mixture was diluted with water and purified using HPLC to get compound 2. To deprotect -Boc group, 2 was dissolved in HCl in dioxane and stirred for about 1 h. Then it was evaporated to dryness and was taken to next step without further purification. Compound 3 (1 equiv) was dissolved in DMF and treated with the appropriate Fmoc-N-amido-PEGn-acid (1.1 equiv), PyBop (1.5 equiv), DIPEA (2 equiv) and stirred for about 12 h. Once the starting materials were consumed, this was purified with HPLC to get 4. Compound 4 (1 equiv) was deprotected with tris(aminoethyl)amine (10 equiv) in DMF and purified again with HPLC (mobile phase A=10 mM Ammonium Acetate, pH=7; Organic phase B=Acetonitrile). Purified compound (1 equiv) was treated with FITC (1.1 equiv) to get the FITC-PEGn-TLR7 non-releasable conjugate in moderate yield.

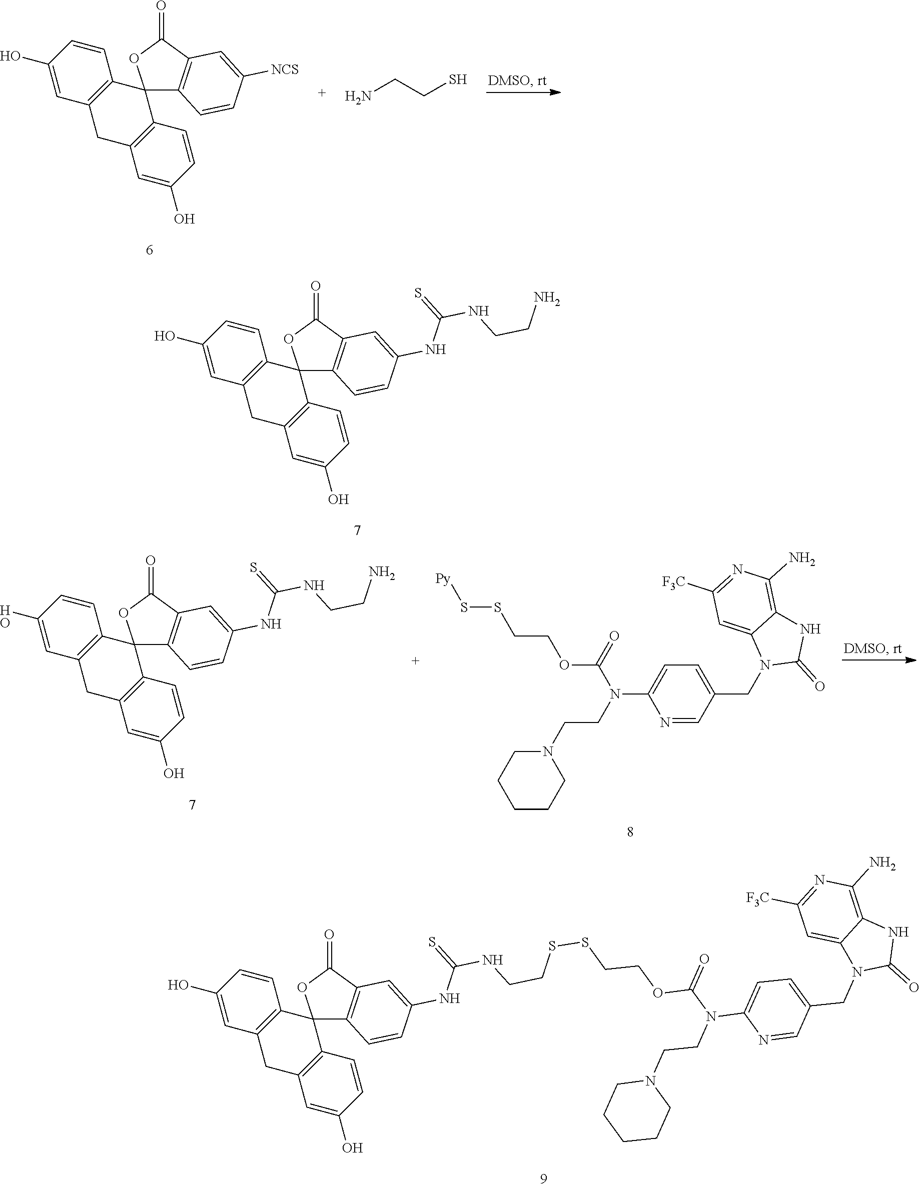

To a stirred solution of FITC (1 equiv) in DMSO, mercapto ethylamine (1 equiv) was added in presence of DIPEA (1 equiv). This was stirred for about 10 min to get compound 7. In another flask, compound 8 (1 equiv) was dissolved in DMSO and mixed with appropriate quantity of intermediate 7 (1 equiv). This was stirred for about 2 h and analyzed with LCMS. The product was purified with HPLC (mobile phase A=10 mM Ammonium Acetate, pH=7; Organic phase B=Acetonitrile) to get the compound 9 in moderate to good yield.

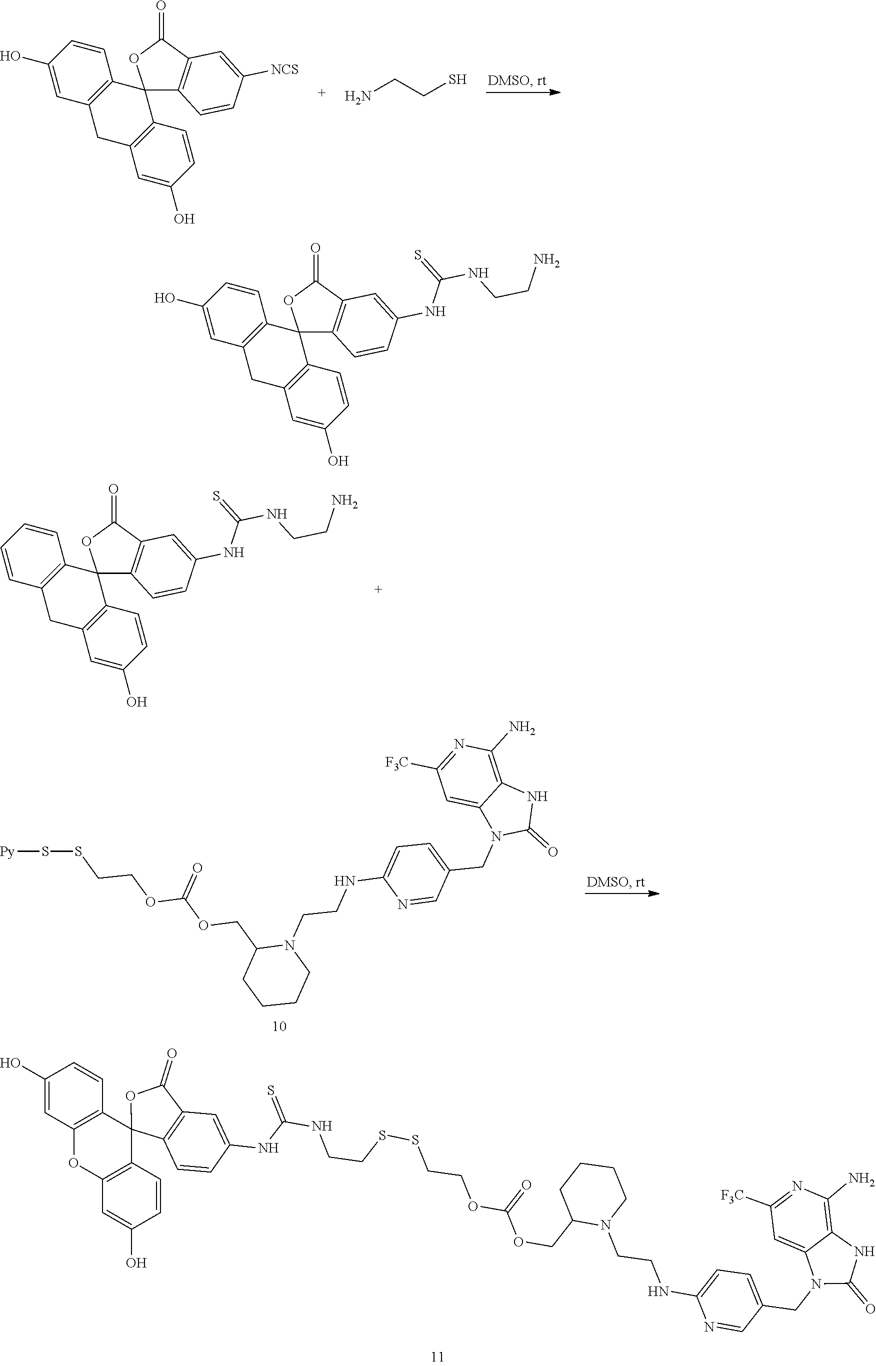

To a stirred solution of compound 10 (1 equiv) in DMSO was added intermediate 7 (1 equiv). This was stirred for about 2 h and analyzed with LCMS. The product was purified with HPLC (mobile phase A=10 mM Ammonium Acetate, pH=7; Organic phase B=Acetonitrile) to get the compound 11(FITC-TLR7-3) in moderate to good yield.

### Synthetic Scheme for FITC-PEGn-TLR7 Non Releasable Conjugate

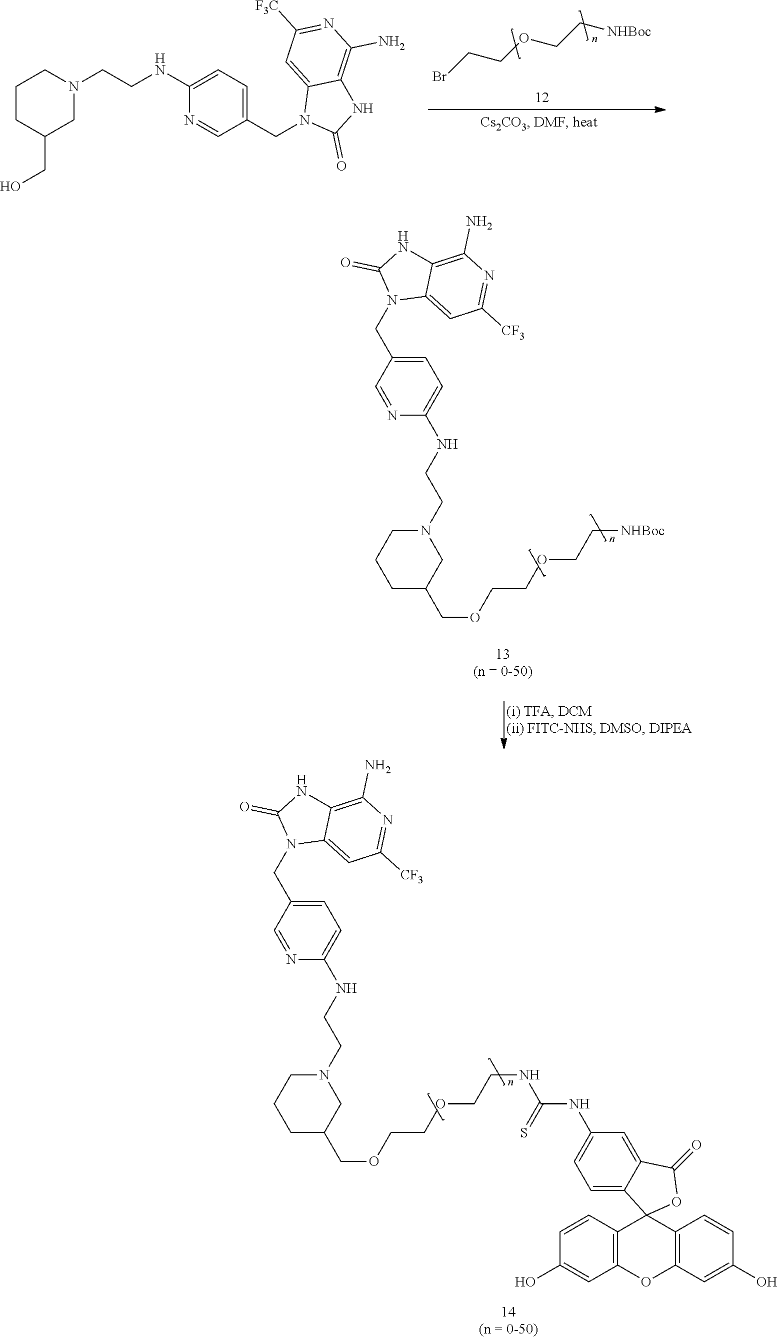

To a stirred solution of compound 1 (1 equiv) in DMF, Cs₂CO₃ (2 equiv) and Boc-bromo amine 12 (1.5 equiv) was added and heated at 70° C. for 5 h. Once the starting materials were consumed, the reaction mixture was diluted with water and purified using HPLC to get compound 13. To deprotect -Boc group, 2 was dissolved in HCl in dioxane and stirred for about 1 h. Then it was evaporated to dryness and was taken to next step without further purification. Deprotected compound 13 (1 equiv) was treated with FITC (1.1 equiv) to get the FITC-PEGn-TLR7 (14) non-releasable conjugate in moderate to good yields.

### Example 18

### Design of FITC-TLR7 Agonist Rejuvenating Compounds Linkers

In this Example, the design of FITC-TLR7 agonist rejuvenating compounds with linkers is shown.

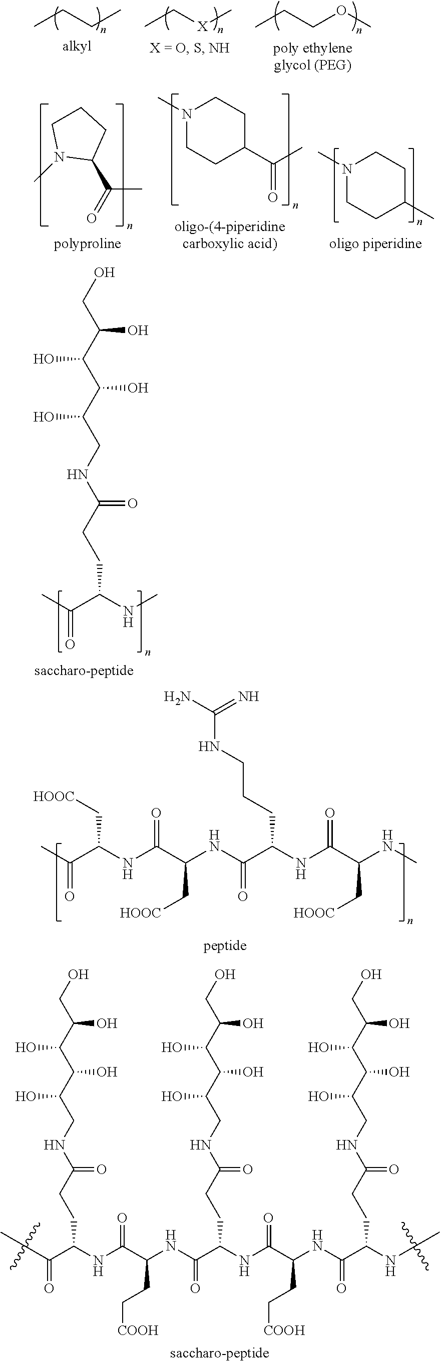

### Example 19

### Rejuvenation of Anti-FITC CAR-Cells In Vitro

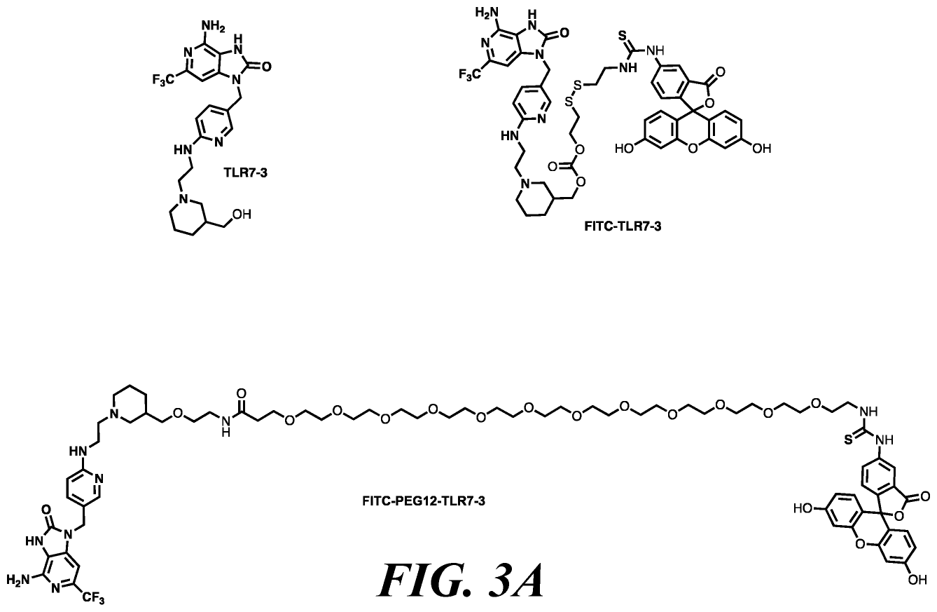

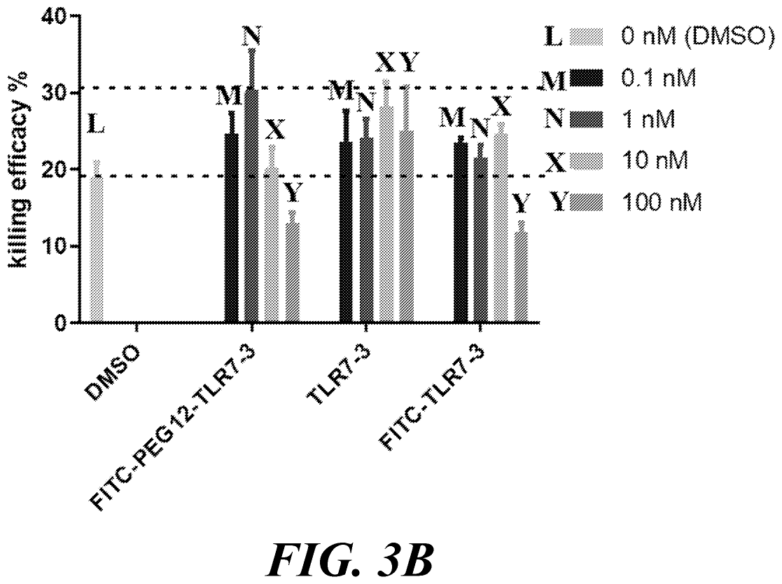

In this example, rejuvenation of anti-FITC CAR-T cells by FITC conjugated to a TLR7 agonist (rejuvenating compound) is shown in a CAR-T cell exhaustion model. Structures of TLR7 agonists were described in FIG. 3A. Briefly, the data in FIG. 3B shows the rejuvenation effect of the TLR7 agonist and TLR7-agonist-FITC conjugate (rejuvenating compound) on exhausted anti-FITC CAR-T cells in vitro in the exhaustion model, indicated by increased killing.

### Example 20

### Rejuvenation of Anti-FITC CAR-Cells In Vivo

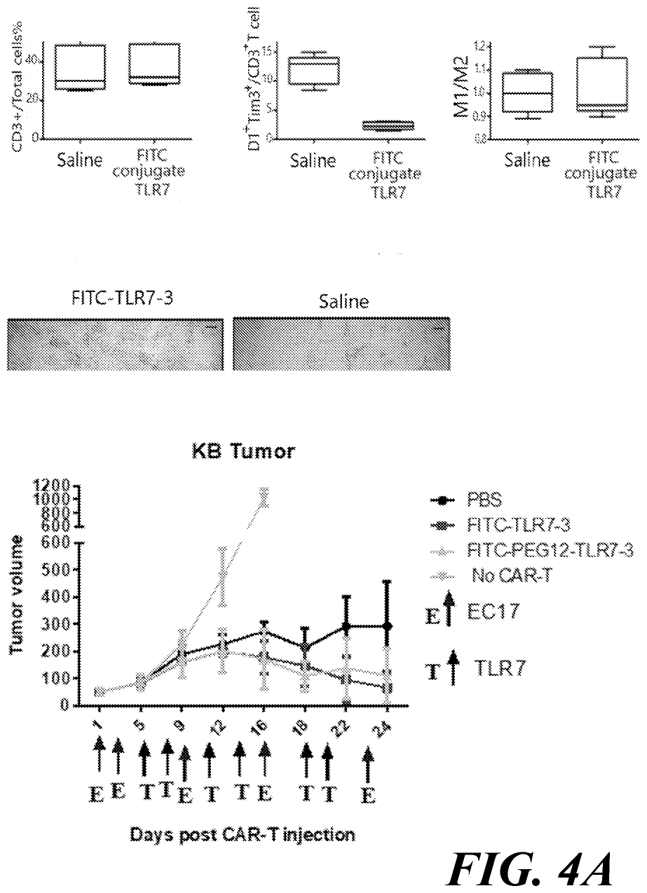

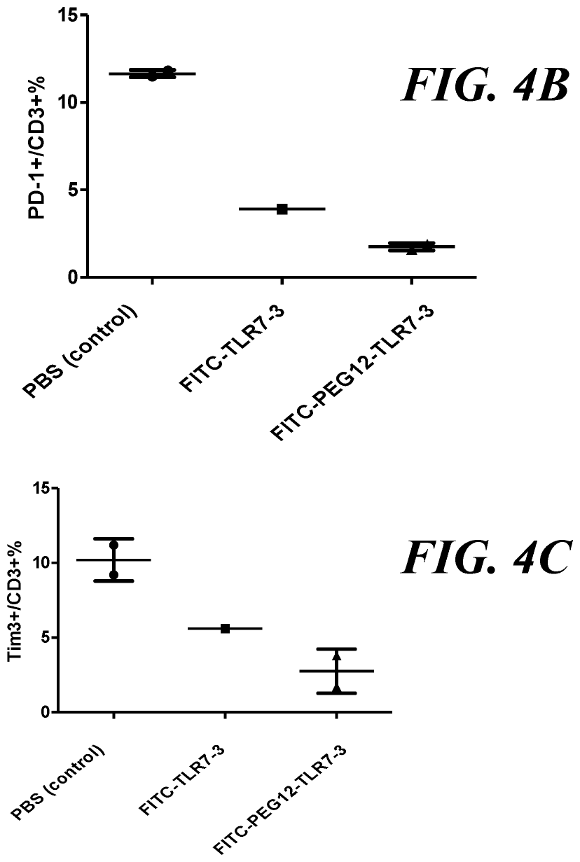

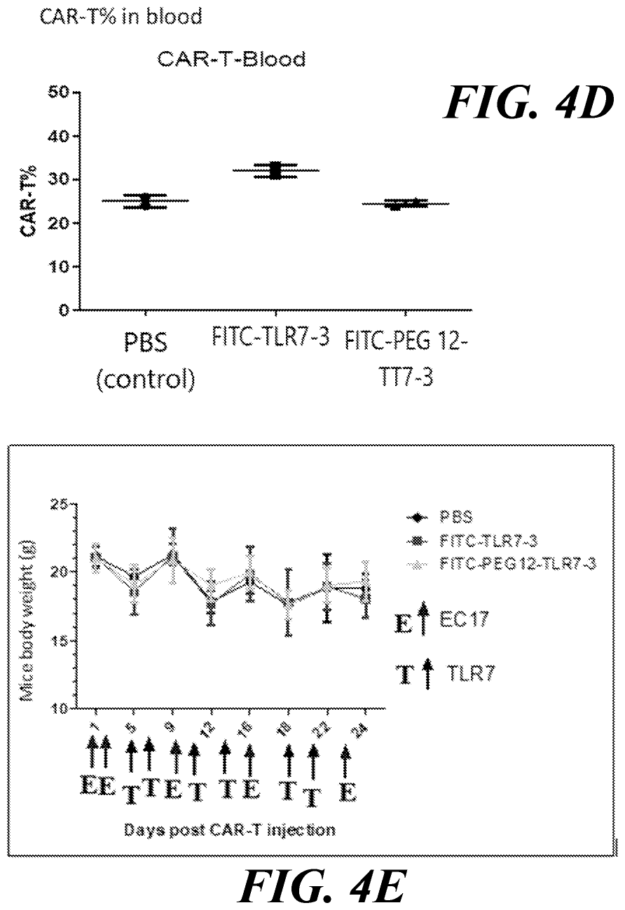

In this example, rejuvenation of anti-FITC CAR-T cells by a TLR7 agonist conjugated to FITC (rejuvenating compound) in a KB xenograft model as described above is shown. Briefly, FIG. 4 shows the rejuvenation effect of the FITC-TLR7 agonist conjugate in the KB xenograft model, as indicated by the decreased tumor size (FIG. 4A) and decreased exhaustion marker expression (PD-1+Tim3+) (FIGS. 4B and 4C). FIGS. 4D and 4E shows the change in the percentage of CAR T cells and change in the mice body weight during treatment.

1-51. (canceled)

52. A combination for modifying T cell activity in a subject with cancer, wherein the combination comprises:
one or more adaptor compounds, or pharmaceutically acceptable salts thereof, wherein each adaptor compound, or pharmaceutically acceptable salt thereof, comprises a small molecule ligand linked to a first targeting moiety; and
an activity modify compound linked to a second targeting moiety, the activity modify compound comprising:
a rejuvenating compound, or a pharmaceutically acceptable salt thereof, or
an immunosuppressive compound, or a pharmaceutically acceptable salt thereof.

53-55. (canceled)

56. The combination of claim 52, wherein at least one of the one or more adaptor compounds, or pharmaceutically acceptable salts thereof, further comprises a first linker positioned between the small molecule ligand and the first targeting moiety.

57. The combination of claim 52, wherein a second linker is positioned between the second targeting moiety and the activity modifying compound.

58. (canceled)

59. The combination of claim 52, further comprising a composition comprising:
a vector comprising a promoter operatively linked to a nucleic acid sequence encoding a chimeric antigen receptor (CAR); or
T cells expressing the CAR (CAR-T cells);
wherein the CAR is directed to the first targeting moiety, the second targeting moiety, or both the first and second targeting moieties.

60. The combination of claim 59, wherein the CAR has a recognition region comprising a single chain fragment variable (scFv) region of an antibody that binds to the first targeting moiety and the second targeting moiety with high affinity.

61. The combination of claim 60, wherein the first and second targeting moieties bind to the scFv region with an affinity in the sub-nanomolar range.

62. The combination of claim 52, wherein the small molecule ligand is selected from a group consisting of a folate, a 2-[3-(1,3-dicarboxypropyl)ureido]pentanedioic acid (DUPA) ligand, a neurokinin 1 receptor (NK-1R) ligand, a carbonic anhydrase IX (CAIX) ligand, a ligand of gamma glutamyl transpeptidase, a natural killer group 2D receptor (NKG2D) ligand, and a cholecystokinin B receptor (CCKBR or CCK2) ligand.

63. The combination of claim 52, wherein the first targeting moiety, the second targeting moiety, or both the first and second targeting moieties are independently selected from a group consisting of 2,4-dinitrophenol (DNP), 2,4,6-trinitrophenol (TNP), biotin, digoxigenin, fluorescein, fluorescein isothiocyanate (FITC), NHS-fluorescein, pentafluorophenyl ester, tetrafluorophenyl ester, a knottin, a centyrin, and a DARPin.

64-65. (canceled)

66. The combination of claim 56, wherein:
a second linker is positioned between the second targeting moiety and the activity modifying compound and the first linker and the second linker have the same structure or different structures; and
the first linker, the second linker, or both the first and second linkers:
each independently comprises a C₁-C₂₀ alkyl, a polyethylene glycol (PEG), a polyproline, an oligo-(4-piperidine carboxylic acid, an oligo piperidine, a peptide, a saccharo-peptide, a hydrophilic amino acid, a sugar, an unnatural peptidoglycan, a polyvinylpyrrolidone, pluronic F-127, or a combination thereof, and/or
at least one of the first linker and the second linker comprises a structure having the formula: 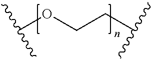
wherein n is an integer from 0 to 200.

67-68. (canceled)

69. The combination of claim 52, wherein the activity modifying compound comprises a rejuvenating compound, or the pharmaceutically acceptable salt thereof, selected from a group comprising a Toll-Like Receptor (TLR) agonist, a stimulator of interferon genes agonist, and a phosphatase inhibitor, a Nod-like receptor stimulant, an absent in melanoma 2-like receptor agonist, a kinase inhibitor, a retinoic acid-inducible gene-I-like receptor, and a receptor for advanced glycation end products.

70. The combination of claim 69, wherein the rejuvenating compound, or the pharmaceutically acceptable salt thereof, is a TLR7 agonist.

71. The combination of claim 70, wherein the TLR7 agonist has the formula

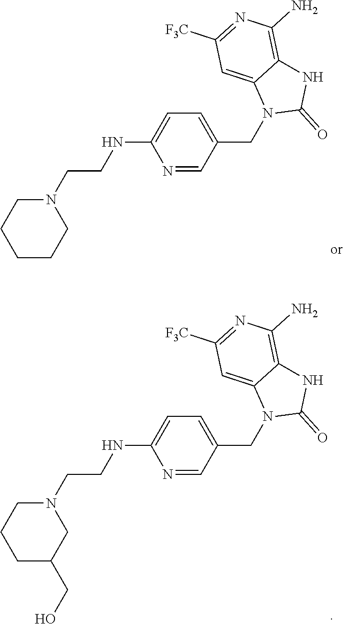 [SMILES: Nc1nc(C(F)(F)F)cc2c1[nH]c(=O)n2Cc1ccc(NCCN2CCCCC2)nc1 ; Nc1nc(C(F)(F)F)cc2c1[nH]c(=O)n2Cc1ccc(NCCN2CCCC(CO)C2)nc1]

72. The combination of claim 56, wherein the first linker in the one or more adaptor compounds, or the pharmaceutically acceptable salts thereof, is positioned between the small molecule ligand and the first targeting moiety and comprises one or more structures selected from the following formulae: 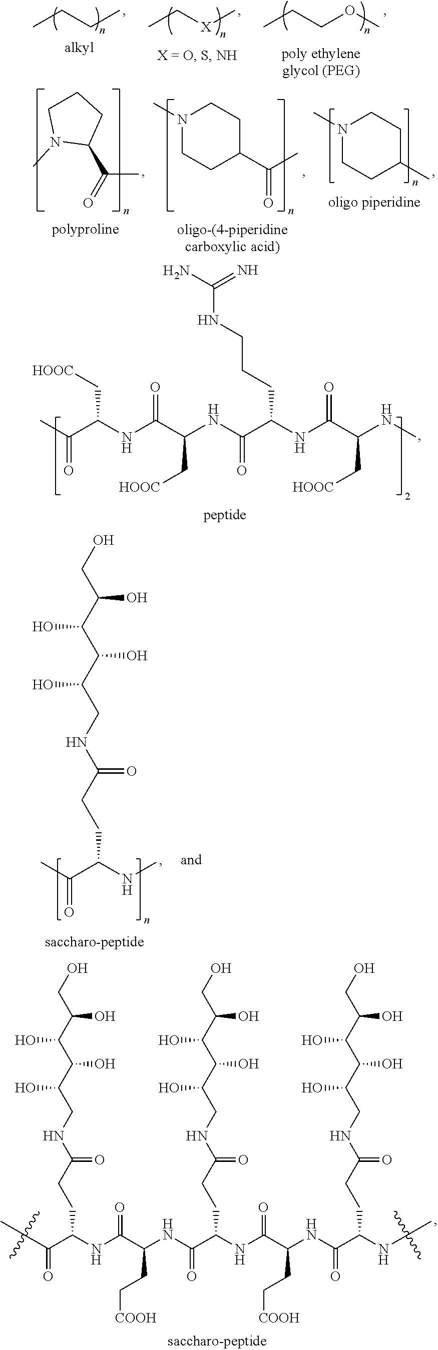
wherein n is an integer from 0 to 200.

73. The combination of claim 57, wherein the second linker is positioned between the second targeting moiety and the activity modifying compound, or the pharmaceutically acceptable salt thereof, and comprises one or more structures selected from the following formulae: 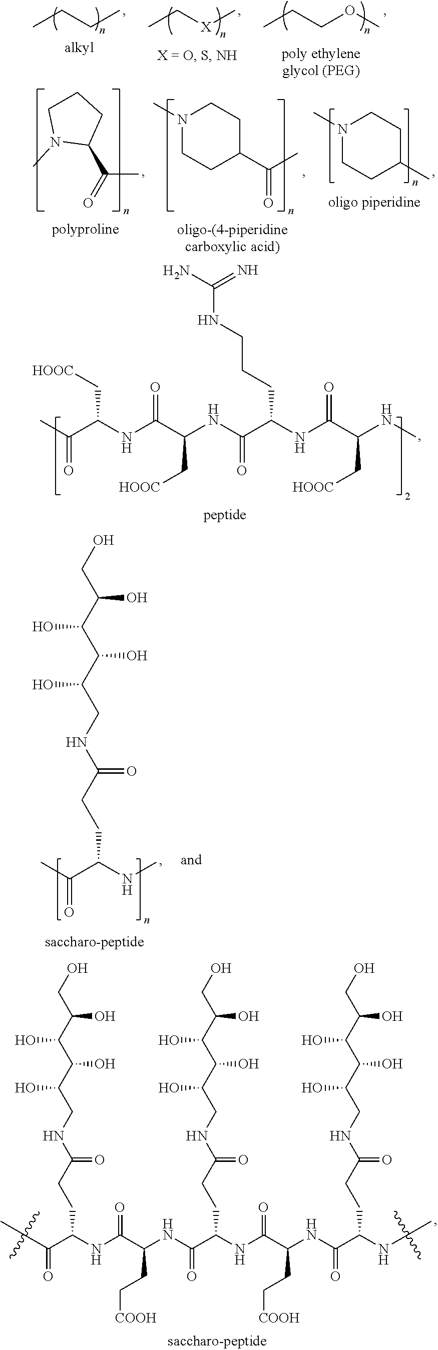
wherein n is an integer from 0 to 200.

74. The combination of claim 52, wherein the activity modifying compound comprises a rejuvenating compound, or the pharmaceutically acceptable salt thereof, that has the formula:

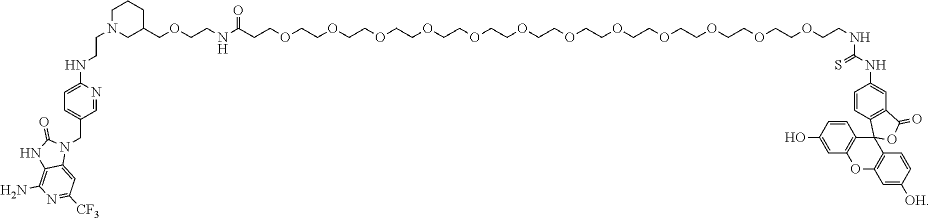 [SMILES: Nc1nc(C(F)(F)F)cc2c1[nH]c(=O)n2Cc1ccc(NCCN2CCCC(COCCNC(=O)CCOCCOCCOCCOCCOCCOCCOCCOCCOCCOCCOCCOCCNC(=S)Nc3ccc4c(c3)C(=O)OC43c4ccc(O)cc4Oc4cc(O)ccc43)C2)nc1]

75. The combination of claim 52, wherein the activity modifying compound comprises a rejuvenating compound, or the pharmaceutically acceptable salt thereof, that has a structure of one of the following formulae: 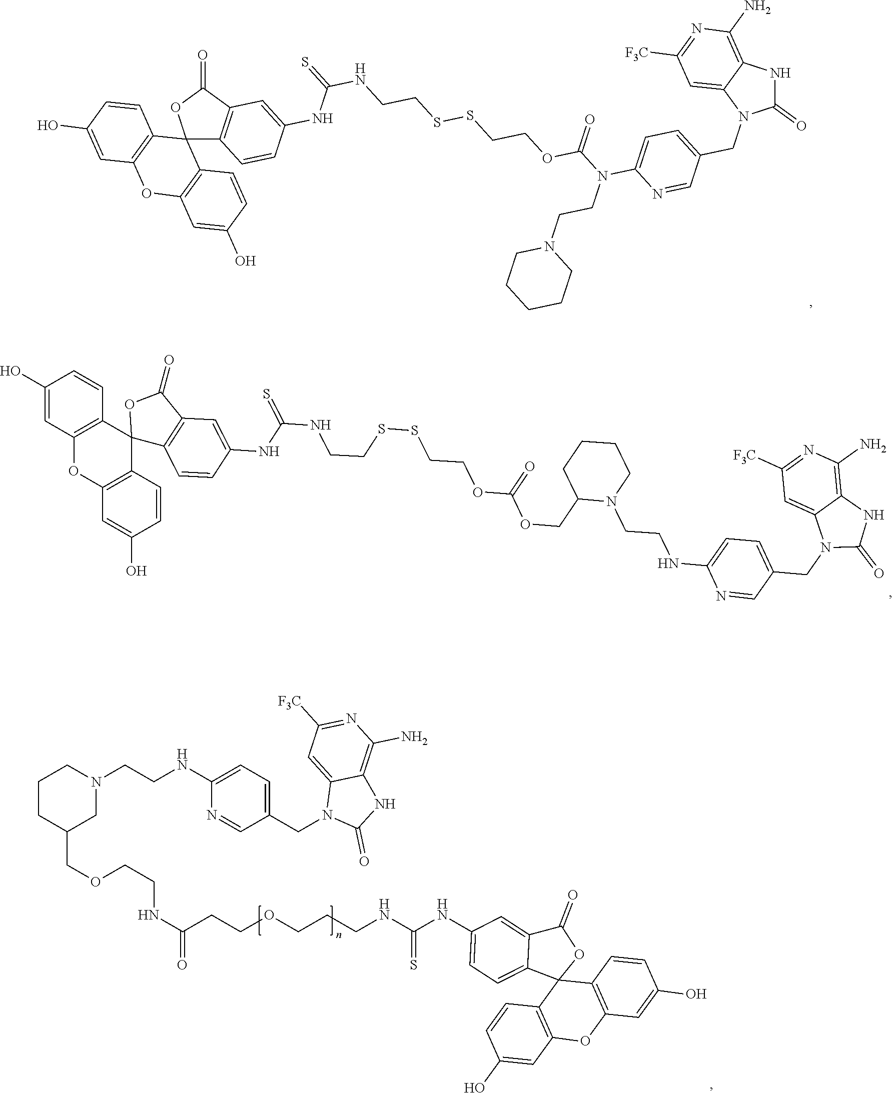
wherein n=0 to 200, and 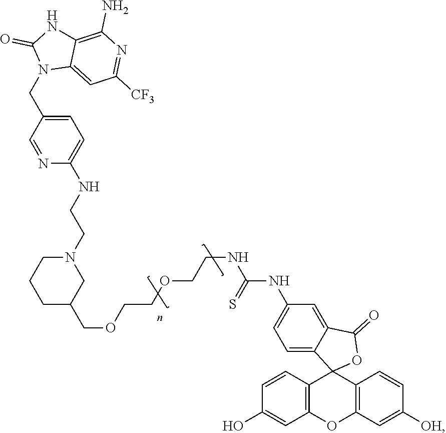
wherein n=0 to 50.

76. The combination of claim 60, wherein the scFv region of the antibody is a scFv region of an anti-FITC antibody.

77-79. (canceled)

80. The combination of claim 52, wherein each adaptor compound, or the pharmaceutically acceptable salt thereof, is not an antibody, and does not comprise a fragment of an antibody.

81. The combination of claim 52, wherein the first targeting moiety does not comprise a peptide epitope.

82-87. (canceled)

88. The combination of claim 52, wherein the small molecule ligand of the one or more adaptor compounds, or the pharmaceutical acceptable salts thereof, comprises a structure having the formula: 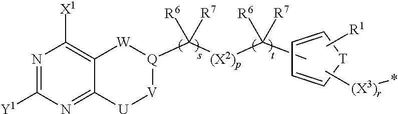
wherein:
X¹ and Y¹ are each independently selected from the group consisting of a halo, R², OR², SR³, and NR⁴R⁵;
U, V, and W represent divalent moieties each independently selected from the group consisting of —(R^{6a})C═, —N═, —(R^{6a})C(R^{7a})—, and —N(R^{4a})—;
Q is selected from the group consisting of C and CH;
T is selected from the group consisting of S, O, N, and —C═C—;
X² and X³ are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C≡CH)—, —N(CH₂C≡CH)—, C₁-C₁₂ alkylene, and C₁-C₁₂ alkyeneoxy, where Z is oxygen or sulfur;
R¹ is selected-from the group consisting of hydrogen, halo, C₁-C₁₂ alkyl, and C₁-C₁₂ alkoxy;
R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;
R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group;
R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;
p, r, s, and t are each independently either 0 or 1; and
* represents a covalent bond, if the one or more adaptor compound, or pharmaceutically acceptable salt thereof, comprises a chemical moiety.

89. A method of modifying T cell activity in a subject with cancer and/or treating a cancer, the method comprising:
administering to the subject the combination of claim 52; and
administering to a subject a composition comprising:
a vector comprising a promoter operatively linked to a nucleic acid sequence encoding a chimeric antigen receptor (CAR), or
T cells expressing the CAR (CAR-T cells),
wherein the CAR is directed to the first targeting moiety, the second targeting moiety, or both the first and second targeting moieties.

90. The method of claim 89, wherein the one or more adaptor compounds, or pharmaceutically acceptable salts thereof, of the combination comprise at least:
a first set of adaptor compounds or pharmaceutically acceptable salts thereof, each adaptor compound or pharmaceutically acceptable salt thereof of the first set comprising a first small molecule ligand linked to the first targeting moiety; and
a second set of adaptor compounds or pharmaceutically acceptable salts thereof, each adaptor compound or pharmaceutically acceptable salt thereof of the second set comprising a second small molecule ligand linked to the first targeting moiety;
wherein the first small molecule ligand is specific to a receptor overexpressed on a first type of cancer cell and the second small molecule ligand is specific to a receptor overexpressed on a second type of cancer cell.

91. The method of claim 89, wherein the activity modifying compound of the combination comprises:
a rejuvenating compound, or a pharmaceutically acceptable salt thereof, formulated to rejuvenate exhausted CAR-T cells; or an immunosuppressive compound, or a pharmaceutically acceptable salt thereof, formulated to reduce the activity of the CAR-T cells.

92. The method of claim 89, wherein the activity modifying compound, or the pharmaceutically acceptable salt thereof, of the combination comprises an immunosuppressive compound, or a pharmaceutically acceptable salt thereof, selected from the group consisting of tacrolimus, sirolimus, and cyclosporine.

93. The method of claim 89, wherein the small molecule ligand of the combination is selected from the group consisting of a folate, a 2-[3-(1,3-dicarboxypropyl)ureido]pentanedioic acid (DUPA) ligand, a neurokinin 1 receptor (NK-1R) ligand, a carbonic anhydrase IX (CAIX) ligand, a ligand of gamma glutamyl transpeptidase, a natural killer group 2D receptor (NKG2D) ligand, and a cholecystokinin B receptor (CCKBR or CCK2) ligand.

94. The method of claim 89, wherein the first targeting moiety and the second targeting moiety are each independently selected from the group consisting of 2,4-dinitrophenol (DNP), 2,4,6-trinitrophenol (TNP), biotin, digoxigenin, fluorescein, fluorescein isothiocyanate (FITC), NHS-fluorescein, pentafluorophenyl ester, tetrafluorophenyl ester, knottin, centyrin, and DARPin.

95. The method of claim 89, wherein the activity modifying compound of the combination comprises a rejuvenating compound, or the pharmaceutically acceptable salt thereof, selected from the group consisting of a Toll-Like Receptor (TLR) agonist, a stimulator of interferon genes agonist, a phosphatase inhibitor, a Nod-like receptor stimulant, an absent in melanoma 2-like receptor agonist, a kinase inhibitor, a retinoic acid-inducible gene-I-like receptor, and a receptor for advanced glycation end products.

96. A method of treating a subject having received CAR-T cell therapy comprising:
administering to the subject one or more adaptor compounds, or pharmaceutically acceptable salts thereof, each adaptor compound or pharmaceutically acceptable salt thereof comprising a small molecule ligand linked to a first targeting moiety, wherein, prior to administering the one or more adaptor compounds or pharmaceutically acceptable salts thereof to the subject, the subject has received at least a dose of T cells expressing a chimeric antigen receptor (CAR) that recognizes and binds to the first targeting moiety; and
administering to the subject an activity modifying compound linked to a second targeting moiety, wherein the CAR recognizes and binds to the second targeting moiety.

97. The method of claim 96, wherein the activity modifying compound comprises a rejuvenating compound, or a pharmaceutically acceptable salt thereof, linked to the second targeting moiety, wherein the rejuvenating compound, or the pharmaceutically acceptable salt thereof, is selected from the group comprising a Toll-Like Receptor (TLR) agonist, a stimulator of interferon genes agonist, a phosphatase inhibitor, a Nod-like receptor stimulant, an absent in melanoma 2-like receptor agonist, a kinase inhibitor a retinoic acid-inducible gene-I-like receptor, and a receptor for advanced glycation end products.

98. The method of claim 96, wherein the one or more adaptor compounds, or pharmaceutically acceptable salts thereof and the activity modifying compound comprise the combination of claim 52.


SEQUENCE LISTING (3 sequences)

SEQ ID NO: 1 - DNA, 1554 bp, Artificial Sequence
  Nucleic acid sequence for encoding a chimeric antigen receptor
atggccttaccagtgaccgccttgctcctgccgctggccttgctgctccacgccgccaggccggatgtcgtgatgacccagacccccctcagcctcccagtgtccctcggtgaccaggcttctattagttgcagatccagccagtccctcgtgcactctaacggtaatacctacctgagatggtatctccagaagcccggacagagccctaaggtgctgatctacaaagtctccaaccgggtgtctggagtccctgaccgcttctcagggagcggttccggcaccgacttcaccctgaagatcaaccgggtggaggccgaagacctcggcgtctatttctgctctcagagtacacatgtgccctggaccttcggcggagggaccaagctggagatcaaaagctccgcagacgatgccaagaaagatgccgctaagaaagacgatgctaagaaagacgatgcaaagaaagacggtggcgtgaagctggatgaaaccggaggaggtctcgtccagccaggaggagccatgaagctgagttgcgtgaccagcggattcacctttgggcactactggatgaactgggtgcgacagtccccagagaaggggctcgaatgggtcgctcagttcaggaacaaaccctacaattatgagacatactattcagacagcgtgaagggcaggtttactatcagtagagacgattccaaatctagcgtgtacctgcagatgaacaatctcagggtcgaagatacaggcatctactattgcacaggggcatcctatggtatggagtatctcggtcaggggacaagcgtcacagtcagtttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 2 - protein, 517 aa, Artificial Sequence
  Chimeric antigen receptor amino acid sequence encoded by SEQ ID NO 1 or 2
MALPVTALLLPLALLLHAARPDVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - DNA, 1560 bp, Artificial Sequence
  Nucleic acid sequence for encoding a chimeric antigen receptor
gccaccatggccttaccagtgaccgccttgctcctgccgctggccttgctgctccacgccgccaggccggatgtcgtgatgacccagacccccctcagcctcccagtgtccctcggtgaccaggcttctattagttgcagatccagccagtccctcgtgcactctaacggtaatacctacctgagatggtatctccagaagcccggacagagccctaaggtgctgatctacaaagtctccaaccgggtgtctggagtccctgaccgcttctcagggagcggttccggcaccgacttcaccctgaagatcaaccgggtggaggccgaagacctcggcgtctatttctgctctcagagtacacatgtgccctggaccttcggcggagggaccaagctggagatcaaaagctccgcagacgatgccaagaaagatgccgctaagaaagacgatgctaagaaagacgatgcaaagaaagacggtggcgtgaagctggatgaaaccggaggaggtctcgtccagccaggaggagccatgaagctgagttgcgtgaccagcggattcacctttgggcactactggatgaactgggtgcgacagtccccagagaaggggctcgaatgggtcgctcagttcaggaacaaaccctacaattatgagacatactattcagacagcgtgaagggcaggtttactatcagtagagacgattccaaatctagcgtgtacctgcagatgaacaatctcagggtcgaagatacaggcatctactattgcacaggggcatcctatggtatggagtatctcggtcaggggacaagcgtcacagtcagtttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa